SEQUENCE LISTING (750 sequences)

SEQ ID NO: 1 - protein, 40 aa, Enterococcus faecalis
WLPPAGLLGRCGRWFRPWLLWLQSGAQYKWLGNLFGLGPK

SEQ ID NO: 2 - protein, 5 aa, synthetic construct
  REGION 1..5: note = N-terminal motif of class terminal IIa
  VARIANT 3: note = Xaa = any amino acid
YGXGV

SEQ ID NO: 3 - protein, 135 aa, synthetic construct
  REGION 1..135: note = Hybrid bacteriocin Ent35-MccV
GKYYGNGVSCNKKGCSVDWGRAIGIIGNNSAANLATGGAAGWKSGGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 4 - protein, 46 aa, Lactobacillus acidophilus
MISSHQKTLTDKELALISGGKTHYPTNAWKSLWKGFWESLRYTDGF

SEQ ID NO: 5 - DNA, 141 bp, Lactobacillus acidophilus
  source 1..141: mol_type = genomic DNA
atgatttcatctcatcaaaaaacgttaactgataaagaattagcattaatttctggggggaaaacgcactacccgactaatgcatggaaaagtctttggaaaggtttctgggaaagccttcgttatactgacggtttttag

SEQ ID NO: 6 - protein, 81 aa, Lactobacillus acidophilus
MISMISSHQKTLTDKELALISGGKTYYGTNGVHCTKKSLWGKVRLKNVIPGTLCRKQSLPIKQDLKILLGWATGAFGKTFH

SEQ ID NO: 7 - DNA, 246 bp, Lactobacillus acidophilus
  source 1..246: mol_type = genomic DNA
atgatttcaatgatttcatctcatcaaaaaacgttaactgataaagaattagcattaatttctggggggaaaacgtactatggtactaatggtgtgcattgtactaaaaagagtctttggggtaaagtacgcttaaaaaacgtgattcctggaactctttgtcgtaagcaatcgttgccgatcaaacaggatttaaaaattttactgggctgggctacaggtgcttttggcaagacatttcattaa

SEQ ID NO: 8 - protein, 60 aa, Lactobacillus acidophilus
MDKKTKILFEVLYIICIIGPQFILFVTAKNNMYQLVGSFVGIVWFSYIFWYIFFKQHKKM

SEQ ID NO: 9 - DNA, 183 bp, Lactobacillus acidophilus
  source 1..183: mol_type = genomic DNA
atggataagaaaacaaaaatattatttgaagtattatacatcatctgtataataggccctcaatttatattatttgtgactgcaaaaaacaatatgtatcagttggtgggttcgtttgttggaatagtatggttttcgtatattttttggtatatttttttcaaacaacataaaaaaatgtag

SEQ ID NO: 10 - protein, 65 aa, Lactobacillus gasseri
MALKTLEKHELRNVMGGNKWGNAVIGAATGATRGVSWCRGFGPWGMTACALGGAAIGGYLGYKSN

SEQ ID NO: 11 - DNA, 198 bp, Lactobacillus gasseri
  source 1..198: mol_type = genomic DNA
atggctttaaaaacattagaaaaacatgaattaagaaatgtaatgggtggaaacaagtgggggaatgctgtaataggagctgctacgggagctactcgcggagtaagttggtgcagaggattcggaccatggggaatgactgcctgtgcgttaggaggtgctgcaattggaggatatctgggatataagagtaattaa

SEQ ID NO: 12 - protein, 51 aa, Staphylococcus aureus [sequence omitted]

SEQ ID NO: 13 - DNA, 156 bp, Staphylococcus aureus
  source 1..156: mol_type = genomic DNA
atgagttggttaaattttttaaaatacatcgctaaatatggcaaaaaagcggtatctgctgcttggaagtacaaaggtaaagtattagaatggcttaatgttggtcctactcttgaatgggtatggcaaaaattaaagaaaattgctggattataa

SEQ ID NO: 14 - protein, 61 aa, Enterococcus avium
MTRSKKLNLREMKNVVGGTYYGNGVSCNKKGCSVDWGKAISIIGNNSAANLATGGAAGWKS

SEQ ID NO: 15 - DNA, 186 bp, Enterococcus avium
  source 1..186: mol_type = genomic DNA
atgacaagatcaaaaaaattaaatttacgcgaaatgaagaatgttgttggtggtacctactatggaaatggtgtatcttgtaacaagaaaggctgttcagttgactggggcaaagccatcagtattataggaaataattccgcagcaaacttagcaactggtggtgctgctggttggaagtcataa

SEQ ID NO: 16 - protein, 67 aa, Enterococcus faecalis
MKKKLVICGIIGIGFTALGTNVEAATYYGNGLYCNKQKCWVDWNKASREIGKIIVNGWVQHGPWAPR

SEQ ID NO: 17 - DNA, 204 bp, Enterococcus faecalis
  source 1..204: mol_type = genomic DNA
atgaaaaagaaattagttatttgtggcattattgggattggttttacagcattaggaacaaatgtagaagctgctacgtattacggaaatggtttatattgtaataagcaaaaatgttgggtagactggaataaagcttcaagggaaattggaaaaattattgttaatggttgggtacaacatggcccttgggctcctagatag

SEQ ID NO: 18 - protein, 51 aa, Lactococcus lactis
MKEQNSFNLLQEVTESELDLILGAKGGSGVIHTISHEVIYNSWNFVFTCCS

SEQ ID NO: 19 - DNA, 156 bp, Lactococcus lactis
  source 1..156: mol_type = genomic DNA
atgaaagaacaaaactcttttaatcttcttcaagaagtgacagaaagtgaattggaccttattttaggtgcaaaaggcggcagtggagttattcatacaatttctcatgaagtaatatataatagctggaactttgtatttacttgctgctcttaa

SEQ ID NO: 20 - protein, 74 aa, Enterococcus faecium
MKKKVLKHCVILGILGTCLAGIGTGIKVDAATYYGNGLYCNKEKCWVDWNQAKGEIGKIIVNGWVNHGPWAPRR

SEQ ID NO: 21 - DNA, 225 bp, Enterococcus faecium
  source 1..225: mol_type = genomic DNA
atgaaaaagaaagtattaaaacattgtgttattctaggaatattaggaacttgtctagctggcatcggtacaggaataaaagttgatgcagctacttactatggaaatggtctttattgtaacaaagaaaaatgttgggtagattggaatcaagctaaaggagaaattggaaaaattattgttaatggttgggttaatcatggtccatgggcacctagaaggtag

SEQ ID NO: 22 - protein, 50 aa, Clostridium botulinum
MQKPEIISADLGLCAVNEFVALAAIPGGAATFAVCQMPNLDEIVSNAAYV

SEQ ID NO: 23 - DNA, 153 bp, Clostridium botulinum
  source 1..153: mol_type = genomic DNA
atgcaaaaaccagaaattattagtgctgatttagggctttgtgcagttaatgaatttgtagctcttgctgccattcctggtggtgctgctacatttgcagtatgccaaatgccaaacttggatgagattgttagtaatgcagcatatgtttaa

SEQ ID NO: 24 - protein, 58 aa, Streptococcus equinus
MMNATENQIFVETVSDQELEMLIGGADRGWIKTLTKDCPNVISSICAGTIITACKNCA

SEQ ID NO: 25 - DNA, 177 bp, Streptococcus equinus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcttgtaaaaattgtgcttaa

SEQ ID NO: 26 - protein, 77 aa, Brochothrix campestris
MHKVKKLNNQELQQIVGGYSSKDCLKDIGKGIGAGTVAGAAGGGLAAGLGAIPGAFVGAHFGVIGGSAACIGGLLGN

SEQ ID NO: 27 - DNA, 234 bp, Brochothrix campestris
  source 1..234: mol_type = genomic DNA
atgcacaaggtaaaaaaattaaacaatcaagagttacaacagatcgtgggaggttacagttcaaaagattgtctaaaagatattggtaaaggaattggtgctggtacagtagctggggcagccggcggtggcctagctgcaggattaggtgctatcccaggagcattcgttggagcacattttggagtaatcggcggatctgccgcatgcattggtggattattaggtaactag

SEQ ID NO: 28 - protein, 80 aa, Butyrivibrio fibrisolvens
MSKKQIMSNCISIALLIALIPNIYFIADKMGIQLAPAWYQDIVNWVSAGGTLTTGFAIIVGVTVPAWIAEAAAAFGIASA

SEQ ID NO: 29 - DNA, 243 bp, Butyrivibrio fibrisolvens
  source 1..243: mol_type = genomic DNA
atgagtaaaaaacaaattatgagtaactgtatatcaattgcattattaatagcactaattcctaatatctattttattgcagataaaatgggaattcagttagcacctgcttggtatcaagatattgtgaattgggtatctgctggtggaacacttactactggttttgcgattattgtaggagttacagtaccggcatggatagcagaagcagctgcagcttttggtatagcttcagcatga

SEQ ID NO: 30 - protein, 48 aa, Butyrivibrio fibrisolvens
MNKELNALTNPIDEKELEQILGGGNGVIKTISHECHMNTWQFIFTCCS

SEQ ID NO: 31 - DNA, 147 bp, Butyrivibrio fibrisolvens
  source 1..147: mol_type = genomic DNA
atgaacaaagaacttaatgcacttacaaatcctattgacgagaaggagcttgagcagatcctcggtggtggcaatggtgtcatcaagacaatcagccacgagtgccacatgaacacatggcagttcattttcacatgttgctcttaa

SEQ ID NO: 32 - protein, 66 aa, Carnobacterium maltaromaticum
MNSVKELNVKEMKQLHGGVNYGNGVSCSKTKCSVNWGQAFQERYTAGINSFVSGVASGAGSIGRRP

SEQ ID NO: 33 - DNA, 201 bp, Carnobacterium maltaromaticum
  source 1..201: mol_type = genomic DNA
atgaatagcgtaaaagaattaaacgtgaaagaaatgaaacaattacacggtggagtaaattatggtaatggtgtttcttgcagtaaaacaaaatgttcagttaactggggacaagcctttcaagaaagatacacagctggaattaactcatttgtaagtggagtcgcttctggggcaggatccattggtaggagaccgtaa

SEQ ID NO: 34 - protein, 61 aa, Carnobacterium maltaromaticum
MKSVKELNKKEMQQINGGAISYGNGVYCNKEKCWVNKAENKQAITGIVIGGWASSLAGMGH

SEQ ID NO: 35 - DNA, 186 bp, Carnobacterium maltaromaticum
  source 1..186: mol_type = genomic DNA
atgaaaagcgttaaagaactaaataaaaaagaaatgcaacaaattaatggtggagctatctcttatggcaatggtgtttattgtaacaaagagaaatgttgggtaaacaaggcagaaaacaaacaagctattactggaatagttatcggtggatgggcttctagtttagcaggaatgggacattaa

SEQ ID NO: 36 - protein, 71 aa, Carnobacterium maltaromaticum
MNNVKELSIKEMQQVTGGDQMSDGVNYGKGSSLSKGGAKCGLGIVGGLATIPSGPLGWLAGAAGVINSCMK

SEQ ID NO: 37 - DNA, 216 bp, Carnobacterium maltaromaticum
  source 1..216: mol_type = genomic DNA
atgaataatgtaaaagagttaagtattaaagaaatgcaacaagttactggtggagaccaaatgtcagatggtgtaaattatggaaaaggctctagcttatcaaaaggtggtgccaaatgtggtttagggatcgtcggcggattagctactatcccttcaggtcctttaggctggttagccggagcagcaggtgtaattaatagctgtatgaaataa

SEQ ID NO: 38 - protein, 64 aa, Carnobacterium maltaromaticum
MLYELVAYGIAQGTAEKVVSLINAGLTVGSIISILGGVTVGLSGVFTAVKAAIAKQGIKKAIQL

SEQ ID NO: 39 - DNA, 195 bp, Carnobacterium maltaromaticum
  source 1..195: mol_type = genomic DNA
atgttatatgaattagttgcatatggtatcgcacaaggtacagctgaaaaggttgtaagtctaattaacgcaggtttaacagtagggtctattatttcaattttgggtggggtcacagtcggtttatcaggtgtcttcacagcagttaaagcagcaattgctaaacaaggaataaaaaaagcaattcaattataa

SEQ ID NO: 40 - protein, 836 aa, Pectobacterium carotovorum
  source 1..836: note = subsp. carotovorum
MIKYRLYAPNDGDTMTVSGGGGWVSNDDRKGGNDRDNGKGGSAVDFSKNPEKQAIVNPYLAIAIPMPVYPLYGKLGFTINTTAIETELANVRAAINTKLATLSAVIGRSLPVVGRVFGVTAAGMWPSSTAPSSLDSIYNQAHQQALAQLAAQQGVLNKGYNVTAMPAGFVSSLPVSEIKSLPTAPASLLAQSVINTELSQRQLALTQPTTNAPVANIPVVKAEKTAMPGVYSAKIIAGEPAFQIKVDNTKPALAQNPPKVKDDIQVSSFLSSPVADTHHAFIDFGSDHEPVYVSLSKIVTAEEEKKQVEEAKRREQEWLLRHPITAAERKLTEIRQVISFAQQLKESSVATISEKTKTVAVYQEQVNTAAKNRDNFYNQNRGLLSAGITGGPGYPIYLALWQTMNNFHQAYFRANNALEQESHVLNLARSDLAKAEQLLAENNRLQVETERTLAEEKEIKRNRVNVSTFGTVQTQLSKLLSDFYAVTSLSQSVPSGALASFSYNPQGMIGSGKIVGKDVDVLFSIPVKDIPGYKSPINLDDLAKKNGSLDLPIRLAFSDENGERVLRAFKADSLRIPSSVRGVAGSYDKNTGIFSAEIDGVSSRLVLENPAFPPTGNVGNTGNTAPDYKALLNTGVDVKPVDKITVTVTPVADPVDIDDYIIWLPTASGSGVEPIYVVFNSNPYGGTEKGKYSKRYYNPDKAGGPILELDWKNVKIDHAGVDNVKLHTGRFKASVENKVMIERLENILNGQITATDTDKRFYTHELRELNRYRNLGIKDGEVPSSIQEESAVWNDTHTATLEDYKINEKEQPLYTDAALQAAYEQELKDALGGKHG

SEQ ID NO: 41 - DNA, 2511 bp, Pectobacterium carotovorum
  source 1..2511: mol_type = genomic DNA; note = subsp. carotovorum
atgattaaataccgtttatatgctccaaatgatggagacaccatgacagtgagtggtggtggtggttgggtttcaaacgatgatcgcaaaggtggtaatgacagggacaatggcaaaggtggttctgccgttgattttagtaaaaatccagaaaagcaggctatcgttaatccctatttggcaatcgcgataccgatgccggtctaccctctttatggaaagctagggttcacaataaatacgacggcaattgagactgaactcgcaaatgtcagagcagcaattaacactaaacttgcaacactcagtgcagtgattggcagatcacttccggtcgttgggcgggtatttggtgttactgccgccggaatgtggccttctagtaccgctcccagtagtctcgattctatatacaatcaagcacatcagcaggctttagcccagttagctgctcaacagggagtattaaataaagggtataacgttacagcaatgcctgcaggtttcgtcagcagtttgcctgttagtgaaatcaaatcattgccaacagctcccgccagtttactggcacaaagtgtgattaataccgaactttcccagcgtcaactggctcttactcagcccacgacgaatgcaccagtcgcgaatattcccgtagttaaagcagagaaaacagcaatgccaggtgtgtattcagcgaaaattattgctggtgagcctgcattccaaatcaaggtcgataataccaaacctgctttggcacagaatccgccgaaagtaaaagatgatattcaggtatcttctttcctttcctcgccagtagctgatacgcaccatgcatttattgattttggcagcgatcatgaaccggtatacgtgtctctttcaaagatcgtgacagccgaggaggagaaaaaacaggttgaagaggccaagcgccgtgagcaggagtggttgttgcgtcatccaattacagctgcggagcgaaaattaactgaaatccgccaagtgatctcttttgctcaacagctaaaagaaagctctgtcgcaaccatttcagaaaaaactaaaactgttgcggtttaccaagaacaggtgaataccgctgcaaaaaatcgcgacaatttttataatcaaaatagaggtctgttaagtgcgggtataactgggggaccgggatatcctatttatcttgctttatggcaaacgatgaataactttcatcaggcttatttcagagcaaataatgcattggaacaagagagtcatgttctgaacctggctcgttctgatctggctaaggctgagcaattgcttgctgagaataatcgacttcaggttgaaacggagcgaacgcttgccgaagaaaaagagataaaacgcaacagggttaatgtatcaacatttggcacagtgcaaactcaacttagtaaattgctgtcagatttttatgctgttacatcactttcccaaagtgttccttcgggggcattagcctctttttcatataatccacaagggatgattggcagcggtaagattgttgggaaggatgtcgatgttttattttccatcccagtaaaagatattccgggatataaatctcctattaacttggacgatttagccaagaaaaatggaagtctggatcttcccattcgtctggcattttctgatgagaatggagaaagggttcttcgggcattcaaagcggatagtctgcgaatcccttcgagtgtcagaggtgtagcgggcagttatgacaaaaatacgggtatttttagtgcagaaattgatggtgtttcatctcgccttgtactggaaaacccagcgtttcctccgaccggaaatgtcggtaatacgggtaatactgcacctgactataaagcattactgaatactggtgttgatgttaaacctgttgataaaatcacagttacggtaacaccagttgctgatccagtggatattgatgactatataatctggttgccaactgcgtctggttctggcgtggaacccatttatgtcgtgtttaacagtaatccgtatggtgggacggaaaaaggaaaatatagcaaacgttattataatccagataaggcaggcggtccgatcttggagctggattggaaaaacgttaagattgaccatgcaggtgtggacaatgttaaattacacacagggcgtttcaaagcgtcggttgaaaacaaagtgatgattgaacgtttggaaaacatactgaatggtcaaatcacggccacggatactgacaagcgattctatacgcatgaattaagagagttaaaccgctacagaaatttaggcatcaaagacggtgaagtgcctagtagcattcaagaagaaagcgctgtttggaacgacacacacacagcgacgcttgaagactacaaaattaatgagaaagagcaaccgttgtacactgatgctgctttgcaggcagcctacgaacaggaactcaaagacgcattaggagggaaacatggctaa

SEQ ID NO: 42 - protein, 74 aa, Bacillus cereus
MENLQMLTEEELMEIEGGGWWNSWGKCVAGTIGGAGTGGLGGAAAGSAVPVIGTGIGGAIGGVSGGLTGAATFC

SEQ ID NO: 43 - DNA, 225 bp, Bacillus cereus
  source 1..225: mol_type = genomic DNA
atggaaaacttacaaatgttaactgaagaagaattaatggaaattgaaggtggaggctggtggaatagctggggtaaatgtgttgctggaactatcggtggagctggaactggtggtttaggtggagctgctgcaggttcagctgttccggttattggtactggtattggtggcgctattggtggagttagcggtggccttacaggtgcagctactttttgctaa

SEQ ID NO: 44 - protein, 78 aa, Streptoverticillium griseoverticillatum
MTASILQQSVVDADFRAALLENPAAFGASAAALPTPVEAQDQASLDFWTKDIAATEAFACRQSCSFGPFTFVCDGNTK

SEQ ID NO: 45 - DNA, 237 bp, Streptoverticillium griseoverticillatum
  source 1..237: mol_type = genomic DNA
atgaccgcttccattcttcagcagtccgtcgtggacgccgacttccgcgcggcgctgcttgagaaccccgccgccttcggcgcttccgccgcggccctgcccacgcccgtcgaggcccaggaccaggcgtcccttgacttctggaccaaggacatcgccgccacggaagccttcgcctgccgccagagctgcagcttcggcccgttcaccttcgtgtgcgacggcaacaccaagtaa

SEQ ID NO: 46 - protein, 76 aa, Geobacillus kaustophilus
MSLLALVAGTLGVSQSIATTVVSIVLTGSTLISIILGITAILSGGVDAILEIGWSAFVATVKKIVAERGKAAAIAW

SEQ ID NO: 47 - DNA, 231 bp, Geobacillus kaustophilus
  source 1..231: mol_type = genomic DNA
atgagtttgctggcgcttgttgccgggacgctcggcgtgtcacagtcaatcgcgacgacggttgtttcgattgtgttgaccggctccactctcatttctattattcttgggatcaccgctattttgtcaggtggagtcgacgccattttggaaattgggtggtcagcttttgtcgcgacggtgaaaaaaatagtggcggaacgaggaaaagcggcagcgattgcatggtaa

SEQ ID NO: 48 - protein, 309 aa, Clostridium tyrobutyricum
MRKVFLRSIISTLVMCAFVSSSFSVNADESKPNDEKIINNIENVTTTKDIVKSNKNNIVYLDEGVMSIPLSGRKPIAIKDDNNKEDLTVTLPIKNTGDISKISSNGTILYKNNSSNSSNIALQPKNDGFKALININDKLANKEYEFTFNLPKNSKLISAATYLGKEYDTKEVFVVDKNNIITSIISPAWAKDANGHNVSTYYKIVSNNKLVQVVEFTENTAFPVVADPNWTKIGKCAGSIAWAIGSGLFGGAKLIKIKKYIAELGGLQKAAKLLVGATTWEEKLHAGGYALINLAAELTGVAGIQANCF

SEQ ID NO: 49 - DNA, 930 bp, Clostridium tyrobutyricum
  source 1..930: mol_type = genomic DNA
ttgagaaaagtatttttaagatcaataatttcaacattagttatgtgtgcatttgtttcaagcagcttttcagtaaatgcggatgaaagcaaaccaaatgatgaaaaaataattaataacatagaaaacgttactactactaaagatattgtaaaaagtaataaaaataatattgtatatttagatgaaggtgtaatgagtattccattgtctgggagaaaacccattgctattaaagatgataataataaagaagatttaactgttacattacctattaagaatactggagatatatctaaaattagtagtaatggtactattctgtataaaaataatagtagtaattcatctaatatagctttacaacctaaaaatgatggatttaaggctttaataaatattaatgataagttagctaataaagaatatgaatttacatttaatttacccaaaaacagtaaattaattagtgctgccacatatttgggtaaagaatatgatacaaaagaagtatttgtagtagacaaaaataatataattacgagtattattagtccagcttgggctaaagatgcaaatggacataatgtttctacttattataagatagtatcgaataataaattagtacaagttgttgaattcacagaaaatactgcattcccggtggtagctgatcctaattggactaaaattgggaaatgcgctgggtcaatagcatgggctataggttctggcctttttggtggagcaaagctaattaaaataaaaaaatatatagcagagcttggaggacttcaaaaagcagctaaattattagttggtgcaaccacttgggaagaaaaattacacgcaggcggttatgcattaattaacttagctgctgagctaacaggtgtagcaggtatacaagcaaattgtttttaa

SEQ ID NO: 50 - protein, 62 aa, Bacillus coagulans
MKKIEKLTEKEMANIIGGKYYGNGVTCGKHSCSVDWGKATTCIINNGAMAWATGGHQGTHKC

SEQ ID NO: 51 - DNA, 189 bp, Bacillus coagulans
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacctgcataatcaataatggagctatggcatgggctactggtggacatcaaggtactcataaatgctag

SEQ ID NO: 52 - protein, 490 aa, Escherichia coli
MDKVTDNSPDVESTESTEGSFPTVGVDTGDTITATLATGTENVGGGGGAFGGASESSAAIHATAKWSTAQLKKHQAEQAARAAAAEAALAKAKSQRDALTQRLKDIVNDALRANAARSPSVTDLAHANNMAMQAEAERLRLAKAEQKAREEAEAAEKALREAERQRDEIARQQAETAHLLAMAEAAEAEKNRQDSLDEEHRAVEVAEKKLAEAKAELAKAESDVQSKQAIVSRVAGELENAQKSVDVKVTGFPGWRDVQKKLERQLQDKKNEYSSVTNALNSAVSIRDAKKTEVQNAEIKLKEAKDALEKSQVKDSVDTMVGFYQYITEQYGEKYSRIAQDLAEKAKGSKFNSVDEALAAFEKYKNVLDKKFSKVDRDDIFNALESITYDEWAKHLEKISRALKVTGYLSFGYDVWDGTLKGLKTGDWKPLFVTLEKSAVDFGVAKIVALMFSFIVGAPLGFWGIAIITGIVSSYIGDDELNKLNELLGI

SEQ ID NO: 53 - DNA, 1473 bp, Escherichia coli
  source 1..1473: mol_type = genomic DNA
atggataaagtcactgataattctccagatgtggagagcacagaatctactgaggggtcattcccaactgttggggttgatactggcgatacgattacagcgacgcttgcaactggaactgaaaatgttggtggaggcggtggagcatttggtggggccagtgaaagttctgctgcgatacatgcaaccgctaaatggtctaccgcgcagttgaaaaaacatcaggctgaacaggctgcccgtgctgctgcggctgaggcagcattggcaaaagcgaaatctcagcgtgatgccctgactcaacgtctcaaggatattgttaatgacgctttacgtgctaatgccgctcgtagtccatcagtaactgaccttgctcatgccaataatatggcaatgcaggcagaggctgagcgtttgcgccttgcgaaggcagagcaaaaagcccgtgaagaagctgaagcagcagaaaaagcgctccgggaagcagaacgccaacgtgatgagattgcccgccaacaggctgaaaccgcgcatttgttagcaatggcggaggcagcagaggctgagaaaaatcgacaggattctcttgatgaagagcatcgggctgtggaagtggcagagaagaagctggctgaggctaaagctgaactggcgaaggccgaaagcgatgtacagagtaagcaagcgattgtttccagagttgcaggggagcttgaaaacgctcaaaaaagtgttgatgtgaaggttaccggatttcctggatggcgtgatgttcagaaaaaactggagagacaattgcaggataagaagaatgaatattcgtcagtgacgaatgctcttaattctgctgttagcattagagatgctaaaaaaacagaagttcagaatgctgagataaaattaaaagaagctaaggatgctcttgagaagagtcaggtaaaagactctgttgatactatggttgggttttatcaatatataaccgaacaatatggggaaaaatattccagaatagctcaggatttagctgaaaaggcgaagggtagtaaatttaatagtgttgatgaagcacttgctgcatttgaaaagtataaaaatgtactggataagaaattcagtaaggttgatagggatgatatttttaatgctttagagtctattacttatgatgagtgggccaagcatctagaaaagatctctagggctcttaaggttactggatatttgtctttcgggtatgatgtatgggatggtaccctaaagggattaaaaacaggagactggaagcctttatttgtcactctggagaagagcgcggtagatttcggcgtggcaaaaattgtggcattaatgtttagttttattgttggtgcgcctcttggcttctggggaattgcaattatcacaggtattgtttcttcttacataggggatgatgagttgaacaagcttaatgaattactaggtatttaa

SEQ ID NO: 54 - protein, 522 aa, Escherichia coli
METAVAYYKDGVPYDDKGQVIITLLNGTPDGSGSGGGGGKGGSKSESSAAIHATAKWSTAQLKKTQAEQAARAKAAAEAQAKAKANRDALTQRLKDIVNEALRHNASRTPSATELAHANNAAMQAEDERLRLAKAEEKARKEAEAAEKAFQEAEQRRKEIEREKAETERQLKLAEAEEKRLAALSEEAKAVEIAQKKLSAAQSEVVKMDGEIKTLNSRLSSSIHARDAEMKTLAGKRNELAQASAKYKELDELVKKLSPRANDPLQNRPFFEATRRRVGAGKIREEKQKQVTASETRINRINADITQIQKAISQVSNNRNAGIARVHEAEENLKKAQNNLLNSQIKDAVDATVSFYQTLTEKYGEKYSKMAQELADKSKGKKIGNVNEALAAFEKYKDVLNKKFSKADRDAIFNALASVKYDDWAKHLDQFAKYLKITGHVSFGYDVVSDILKIKDTGDWKPLFLTLEKKAADAGVSYVVALLFSLLAGTTLGIWGIAIVTGILCSYIDKNKLNTINEVLGI

SEQ ID NO: 55 - DNA, 1569 bp, Escherichia coli
  source 1..1569: mol_type = genomic DNA
atggaaaccgcggtagcgtactataaagatggtgttccttatgatgataagggacaggtaattattactcttttgaatggtactcctgacgggagtggctctggcggcggaggtggaaaaggaggcagtaaaagtgaaagttctgcagctattcatgcaactgctaaatggtctactgctcaattaaagaaaacacaggcagagcaggctgcccgggcaaaagctgcagcggaagcacaggcgaaagcaaaggcaaacagggatgcgctgactcagcgcctgaaggatatcgtgaatgaggctcttcgtcacaatgcctcacgtacgccttcagcaacagagcttgctcatgctaataatgcagctatgcaggcggaagacgagcgtttgcgccttgcgaaagcagaagaaaaagcccgtaaagaagcggaagcagcagaaaaggcttttcaggaagcagaacaacgacgtaaagagattgaacgggagaaggctgaaacagaacgccagttgaaactggctgaagctgaagagaaacgactggctgcattgagtgaagaagctaaagctgttgagatcgcccaaaaaaaactttctgctgcacaatctgaagtggtgaaaatggatggagagattaagactctcaattctcgtttaagctccagtatccatgcccgtgatgcagaaatgaaaacgctcgctggaaaacgaaatgaactggctcaggcatccgctaaatataaagaactggatgagctggtcaaaaaactatcaccaagagccaatgatccgcttcagaaccgtcctttttttgaagcaaccagacgacgggttggggccggtaagattagagaagaaaaacaaaaacaggtaacagcatcagaaacacgtattaaccggataaatgctgatataactcagatccagaaggctatttctcaggtcagtaataatcgtaatgccggtatcgctcgtgttcatgaagctgaagaaaatttgaaaaaagcacagaataatctccttaattcacagattaaggatgctgttgatgcaacagttagcttttatcaaacgctgactgaaaaatatggtgaaaaatattcgaaaatggcacaggaacttgctgataagtctaaaggtaagaaaatcggcaatgtgaatgaagctctcgctgcttttgaaaaatacaaggatgttttaaataagaaattcagcaaagccgatcgtgatgctatttttaatgcgttggcatcggtgaagtatgatgactgggctaaacatttagatcagtttgccaagtacttgaagattacggggcatgtttcttttggatatgatgtggtatctgatatcctaaaaattaaggatacaggtgactggaagccactatttcttacattagagaagaaagctgcagatgcaggggtgagttatgttgttgctttactttttagcttgcttgctggaactacattaggtatttggggtattgctattgttacaggaattctatgctcctatattgataagaataaacttaatactataaatgaggtgttagggatttaa

SEQ ID NO: 56 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLNTARNALTRAEQQLTQQKNTPDGKTIVSPEKFPGRSSTNHSIVVSGDPRFAGTIKITTSAVIDNRANLNYLLSHSGLDYKRNILNDRNPVVTEDVEGDKKIYNAEVAEWDKLRQRLLDARNKITSAESAVNSARNNLSARTNEQKHANDALNALLKEKENIRNQLSGINQKIAEEKRKQDELKATKDAINFTTEFLKSVSEKYGAKAEQLAREMAGQAKGKKIRNVEEALKTYEKYRADINKKINAKDRAAIAAALESVKLSDISSNLNRFSRGLGYAGKFTSLADWITEFGKAVRTENWRPLFVKTETIIAGNAATALVALVFSILTGSALGIIGYGLLMAVTGALIDESLVEKANKFWGI

SEQ ID NO: 57 - DNA, 1881 bp, Escherichia coli
  source 1..1881: mol_type = genomic DNA
atgtctgaccctgtacgtattacaaatcccggtgcagaatcgctggggtatgattcagatggccatgaaattatggccgttgatatttatgtaaaccctccacgtgtcgatgtctttcatggtaccccgcctgcatggagttccttcgggaacaaaaccatctggggcggaaacgagtgggttgatgattccccaacccgaagtgatatcgaaaaaagggacaaggaaatcacagcgtacaaaaacacgctcagcgcgcagcagaaagagaatgagaataagcgtactgaagccggaaaacgcctctctgcggcgattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgacgccctccggctgcatacagagagccggatgctgtttgctgatgctgattctcttcgtatatctccccgggaggccaggtcgttaatcgaacaggctgaaaaacggcagaaggatgcgcagaacgcagacaagaaggccgctgatatgcttgctgaatacgagcgcagaaaaggtattctggacacccggttgtcagagctggaaaaaaatggcggggcagcccttgccgttcttgatgcacaacaggcccgtctgctcgggcagcagacacggaatgacagggccatttcagaggcccggaataaactcagttcagtgacggaatcgcttaacacggcccgtaatgcattaaccagagctgaacaacagctgacgcaacagaaaaacacgcctgacggcaaaacgatagtttcccctgaaaaattcccggggcgttcatcaacaaatgattctattgttgtgagcggtgatccgagatttgccggtacgataaaaatcacaaccagcgcagtcatcgataaccgtgcaaacctgaattatcttctgagccattccggtctggactataaacgcaatattctgaatgaccggaatccggtggtgacagaggatgtggaaggtgacaagaaaatttataatgctgaagttgctgaatgggataagttacggcaaagattgcttgatgccagaaataaaatcacctctgctgaatctgcggtaaattcggcgagaaataacctcagtgccagaacaaatgagcaaaagcatgcaaatgacgctcttaatgccctgttgaaggaaaaagagaatatccgtaaccagctttccggcatcaatcagaagatagcggaagagaaaagaaaacaggatgaactgaaggcaacgaaagacgcaattaatttcacaacagagttcctgaaatcagtttcagaaaaatatggtgcaaaagctgagcagttagccagagagatggccgggcaggctaaagggaagaaaatacgtaatgttgaagaggcattaaaaacgtatgaaaagtaccgggctgacattaacaaaaaaattaatgcaaaagatcgtgcagcgattgccgcagcccttgagtctgtgaagctgtctgatatatcgtctaatctgaacagattcagtcggggactgggatatgcaggaaaatttacaagtcttgctgactggatcactgagtttggtaaggctgtccggacagagaactggcgtcctctttttgttaaaacagaaaccatcatagcaggcaatgccgcaacggctcttgtggcactggtcttcagtattcttaccggaagcgctttaggcattatcgggtatggtttactgatggctgtcaccggtgcgctgattgatgaatcgcttgtggaaaaagcgaataagttctggggtatttaa

SEQ ID NO: 58 - protein, 626 aa, Escherichia coli
MSDPVRITNPGAESLGYDSDGHEIMAVDIYVNPPRVDVFHGTPPAWSSFGNKTIWGGNEWVDDSPTRSDIEKRDKEITAYKNTLSAQQKENENKRTEAGKRLSAAIAAREKDENTLKTLRAGNADAADITRQEFRLLQAELREYGFRTEIAGYDALRLHTESRMLFADADSLRISPREARSLIEQAEKRQKDAQNADKKAADMLAEYERRKGILDTRLSELEKNGGAALAVLDAQQARLLGQQTRNDRAISEARNKLSSVTESLKTARNALTRAEQQLTQQKNTPDGKTIVSPEKFPGRSSTNHSIVVSGDPRFAGTIKITTSAVIDNRANLNYLLTHSGLDYKRNILNDRNPVVTEDVEGDKKIYNAEVAEWDKLRQRLLDARNKITSAESAINSARNNVSARTNEQKHANDALNALLKEKENIRSQLADINQKIAEEKRKRDEINMVKDAIKLTSDFYRTIYDEFGKQASELAKELASVSQGKQIKSVDDALNAFDKFRNNLNKKYNIQDRMAISKALEAINQVHMAENFKLFSKAFGFTGKVIERYDVAVELQKAVKTDNWRPFFVKLESLAAGRAASAVTAWAFSVMLGTPVGILGFAIIMAAVSALVNDKFIEQVNKLIGI

SEQ ID NO: 59 - DNA, 1881 bp, Escherichia coli
  source 1..1881: mol_type = genomic DNA
atgtctgaccctgtacgtattacaaatcccggtgcagaatcgctgggatatgattcagatggccatgaaattatggccgttgatatttatgtaaaccctccacgtgtcgatgtctttcatggtaccccgcctgcatggagttccttcgggaacaaaaccatctggggtggaaacgagtgggtcgatgattccccaacccgaagtgatatcgaaaaaagggacaaggaaatcacagcgtacaaaaacacgctcagcgcgcagcagaaagagaatgagaataagcgtactgaagctggaaaacgcctttctgcggcaattgctgcaagggaaaaagatgaaaacacactgaaaacactccgtgccggaaacgcagatgccgctgatattacacgacaggagttcagactcctgcaggcagagctgagagaatacggattccgtactgaaatcgccggatatgatgccctccggctgcatacagagagccggatgctgtttgctgatgctgattctcttcgtatatctccccgcgaggccaggtcgttaatcgaacaggctgaaaaacggcagaaggatgcgcagaacgcagacaagaaggccgctgatatgcttgctgaatacgagcgcagaaaaggtattctggacacgcggttgtcagagctggaaaaaaatggcggggcagcccttgccgttcttgatgcacaacaggcccgtctgctcgggcagcagacacggaatgacagggccatttcagaggcccggaataaactcagttcggtgacggaatcgcttaagacggcccgtaatgcattaaccagagctgaacaacagctgacgcaacagaaaaacacgcctgacggcaaaacgatagtttcccctgaaaaattcccggggcgttcatcaacaaatcattctattgttgtgagtggtgatccgaggtttgccggtacgataaaaatcacaaccagcgcggtcatcgataaccgtgcaaacctgaattatcttctgacccattccggtctggactataaacgcaatattctgaatgaccggaatccggtggtgacagaggatgtggaaggtgacaagaaaatttataatgctgaagttgctgaatgggataagttacggcaacgattgcttgatgccagaaataaaatcacctctgctgaatctgcgataaattcggcgagaaataacgtcagtgccagaacaaatgaacaaaagcatgcaaatgacgctcttaatgccctgttgaaggaaaaagagaatatccgtagccagcttgctgacatcaatcagaaaatagctgaagagaaaagaaaaagggatgaaataaatatggtaaaggatgccataaaactcacctctgatttctacagaacgatatatgatgagttcggtaaacaagcatccgaacttgctaaggagctggcttctgtatctcaagggaaacagattaagagtgtggatgatgcactgaacgcttttgataaattccgtaataatctgaacaagaaatataacatacaagatcgcatggccatttctaaagccctggaagctattaatcaggtccatatggcggagaattttaagctgttcagtaaggcatttggttttaccggaaaagttattgaacgttatgatgttgctgtggagttacaaaaggctgtaaaaacggacaactggcgtccattttttgtaaaacttgaatcactggcagcaggaagagctgcttcagcagttacagcatgggcgttttccgtcatgctgggaacccctgtaggtattctgggttttgcaattattatggcggctgtgagtgcgcttgttaatgataagtttattgagcaggtcaataaacttattggtatctga

SEQ ID NO: 60 - protein, 271 aa, Escherichia coli
METLTVHAPSPSTNLPSYGNGAFSLSAPHVPGAGPLLVQVVYSFFQSPNMCLQALTQLEDYIKKHGASNPLTLQIISTNIGYFCNADRNLVLHPGISVYDAYHFAKPAPSQYDYRSMNMKQMSGNVTTPIVALAHYLWGNGAERSVNIANIGLKISPMKINQIKDIIKSGVVGTFPVSTKFTHATGDYNVITGAYLGNITLKTEGTLTISANGSWTYNGVVRSYDDKYDFNASTHRGIIGESLTRLGAMFSGKEYQILLPGEIHIKESGKR

SEQ ID NO: 61 - DNA, 816 bp, Escherichia coli
  source 1..816: mol_type = genomic DNA
atggaaaccttaactgttcatgcaccatcaccatcaactaacttaccaagttatggcaatggtgcattttctctttcagcaccacatgtgcctggtgctggccctcttttagtccaggttgtttatagttttttccagagtccaaacatgtgtcttcaggctttaactcaacttgaggattacatcaaaaaacatggggccagcaaccctctcacattgcagatcatatcgacaaatattggttacttctgtaacgccgaccgaaatctggttcttcaccctggaataagcgtttatgacgcttaccacttcgcaaaaccagcgccaagtcaatatgactatcgctcaatgaatatgaaacaaatgagcggtaatgtcactacaccaattgtggcgcttgctcactatttatggggtaatggcgctgaaaggagcgttaatatcgccaacattggtcttaaaatttcccctatgaaaattaatcagataaaagacattataaaatctggtgtagtaggcacattccctgtttctacaaagttcacacatgccactggtgattataatgttattaccggtgcatatcttggtaatatcacactgaaaacagaaggtactttaactatctctgccaatggctcctggacttacaatggcgttgttcgttcatatgatgataaatacgattttaacgccagcactcaccgtggcattatcggagagtcgctcacaaggctcggggcgatgttttctggtaaagagtaccagatactgcttcctggtgaaattcacattaaagaaagtggtaagcgataa

SEQ ID NO: 62 - protein, 387 aa, Escherichia coli
MGSNGADNAHNNAFGGGKNPGIGNTSGAGSNGSASSNRGNSNGWSWSNKPHKNDGFHSDGSYHITFHGDNNSKPKPGGNSGNRGNNGDGASAKVGEITITPDNSKPGRYISSNPEYSLLAKLIDAESIKGTEVYTFHTRKGQYVKVTVPDSNIDKMRVDYVNWKGPKYNNKLVKRFVSQFLLFRKEEKEKNEKEALLKASELVSGMGDKLGEYLGVKYKNVAKEVANDIKNFHGRNIRSYNEAMASLNKVLANPKMKVNKSDKDAIVNAWKQVNAKDMANKIGNLGKAFKVADLAIKVEKIREKSIEGYNTGNWGPLLLEVESWIIGGVVAGVAISLFGAVLSFLPISGLAVTALGVIGIMTISYLSSFIDANRVSNINNIISSVIR

SEQ ID NO: 63 - DNA, 51 bp, Escherichia coli
  source 1..51: mol_type = genomic DNA
gcaaatcgagtttcgaatataaataacattatatctagtgttattcgatga

SEQ ID NO: 64 - protein, 103 aa, Escherichia coli
MRTLTLNELDSVSGGASGRDIAMAIGTLSGQFVAGGIGAAAGGVAGGAIYDYASTHKPNPAMSPSGLGGTIKQKPEGIPSEAWNYAAGRLCNWSPNNLSDVCL

SEQ ID NO: 65 - DNA, 312 bp, Escherichia coli
  source 1..312: mol_type = genomic DNA
atgagaactctgactctaaatgaattagattctgtttctggtggtgcttcagggcgtgatattgcgatggctataggaacactatccggacaatttgttgcaggaggaattggagcagctgctgggggtgtggctggaggtgcaatatatgactatgcatccactcacaaacctaatcctgcaatgtctccatccggtttaggaggaacaattaagcaaaaacccgaagggataccttcagaagcatggaactatgctgcgggaagattgtgtaattggagtccaaataatcttagtgatgtttgtttataa

SEQ ID NO: 66 - protein, 58 aa, Enterococcus columbae
MMNATENQIFVETVSDQELEMLIGGAGRGWIKTLTKDCPNVISSICAGTIITACKNCA

SEQ ID NO: 67 - DNA, 177 bp, Enterococcus columbae
  source 1..177: mol_type = genomic DNA
atgatgaatgctactgaaaaccaaatttttgttgagactgtgagtgaccaagaattagaaatgttaattggtggtgcaggtcgtggatggattaagactttaacaaaagattgtccaaatgtgatttcttcaatttgtgcaggtacaattattacagcttgtaaaaattgtgcttaa

SEQ ID NO: 68 - protein, 59 aa, Lactobacillus curvatus
MNNVKELSMTELQTITGGARSYGNGVYCNNKKCWVNRGEATQSIIGGMISGWASGLAGM

SEQ ID NO: 69 - DNA, 180 bp, Lactobacillus curvatus
  source 1..180: mol_type = genomic DNA
atgaataatgtaaaagaattaagtatgacagaattacaaacaattaccggcggtgctagatcatatggcaacggtgtttactgtaataataaaaaatgttgggtaaatcggggtgaagcaacgcaaagtattattggtggtatgattagcggctgggctagtggtttagctggaatgtaa

SEQ ID NO: 70 - protein, 64 aa, Streptomyces sp.
MRSEMTLTSTNSAEALAAQDFANTVLSAAAPGFHADCETPAMATPATPTVAQFVIQGSTICLVC

SEQ ID NO: 71 - DNA, 195 bp, Streptomyces sp.
  source 1..195: mol_type = genomic DNA
gtgcgatctgagatgactcttacgagcacgaattccgctgaggctctggcggcgcaggactttgcgaacaccgttctcagcgcggcggccccgggcttccacgcggactgcgagacgccggccatggccaccccggccacgccgaccgtcgcccagttcgtgatccagggcagcacgatctgcctggtctgctga

SEQ ID NO: 72 - protein, 65 aa, Bacillus halodurans
MVNSKDLRNPEFRKAQGLQFVDEVNEKELSSLAGSGDVHAQTTWPCATVGVSVALCPTTKCTSQC

SEQ ID NO: 73 - DNA, 198 bp, Bacillus halodurans
  source 1..198: mol_type = genomic DNA
atggtaaattcaaaagatttgcgtaatcctgaattccgcaaagcccaaggtctacaattcgttgacgaggtgaacgagaaggaactttcgtctctagctggttcaggagatgtgcatgcacaaacaacttggccttgcgctacagttggtgtctccgtagccttgtgcccaactacaaagtgtacaagccagtgctaa

SEQ ID NO: 74 - protein, 66 aa, Carnobacterium divergens
MKNLKEGSYTAVNTDELKSINGGTKYYGNGVYCNSKKCWVDWGQASGCIGQTVVGGWLGGAIPGKC

SEQ ID NO: 75 - DNA, 201 bp, Carnobacterium divergens
  source 1..201: mol_type = genomic DNA
atgaaaaacttaaaagaaggttcatacactgctgttaatactgatgaattaaaaagtatcaatggtggaacaaaatattatgggaatggcgtttattgcaattctaaaaaatgttgggtagattggggacaagcttcaggttgtatcggtcaaactgttgttggcggatggctaggcggagctataccaggtaaatgctaa

SEQ ID NO: 76 - protein, 63 aa, Carnobacterium divergens
MIKREKNRTISSLGYEEISNHKLQEIQGGKGILGKLGVVQAGVDFVSGVWAGIKQSAKDHPNA

SEQ ID NO: 77 - DNA, 192 bp, Carnobacterium divergens
  source 1..192: mol_type = genomic DNA
atgattaaaagagaaaagaacagaacaatttcttcccttggttatgaagaaatttctaatcataaattgcaagaaatacaaggtggaaaaggaattcttggtaaactaggagtagtacaggcaggagtggattttgtatcaggagtgtgggctggaataaaacagtctgccaaagatcatcctaatgcgtaa

SEQ ID NO: 78 - protein, 75 aa, Carnobacterium divergens
MKKQILKGLVIVVCLSGATFFSTPQQASAAAPKITQKQKNCVNGQLGGMLAGALGGPGGVVLGGIGGAIAGGCFN

SEQ ID NO: 79 - DNA, 228 bp, Carnobacterium divergens
  source 1..228: mol_type = genomic DNA
atgaaaaaacaaattttaaaagggttggttatagttgtttgtttatctggggcaacatttttctcaacaccacaacaagcttctgctgctgcaccgaaaattactcaaaaacaaaaaaattgtgttaatggacaattaggtggaatgcttgctggagctttgggtggacctggcggagttgtgttaggtggtataggtggtgcaatagcaggaggttgttttaattaa

SEQ ID NO: 80 - protein, 72 aa, Enterococcus durans
MQTIKELNTMELQEIIGGENDHRMPYELNRPNNLSKGGAKCAAGILGAGLGAVGGGPGGFISAGISAVLGCM

SEQ ID NO: 81 - DNA, 219 bp, Enterococcus durans
  source 1..219: mol_type = genomic DNA
atgcaaacgatcaaagaattgaacacgatggaattacaagaaataattggaggtgaaaatgaccatcggatgccttacgaattgaaccgtccaaataatttatccaaaggtggggctaagtgtgctgctggaatacttggcgctggactaggcgcagtaggcggtggacctggcggatttattagtgccggaatcagtgctgttcttggttgtatgtaa

SEQ ID NO: 82 - protein, 72 aa, Enterococcus durans
MQTIKELNTMELQKIIGGENDHRMPYELNRPNNLSKGGAKCAAGILGAGLGAVGGGPGGFISAGISAVLGCM

SEQ ID NO: 83 - DNA, 219 bp, Enterococcus durans
  source 1..219: mol_type = genomic DNA
atgcaaacgatcaaagaattgaacacgatggaattacaaaaaataattggaggtgaaaatgaccatcggatgccttacgaattgaaccgtccaaataatttatccaaaggtggagctaagtgcgctgccggaatacttggtgctggattaggcgcagtaggcggtggacctggcggatttattagtgccggaatcagtgctgttcttggttgtatgtaa

SEQ ID NO: 84 - protein, 220 aa, Streptococcus dysgalactiae
  source 1..220: note = subsp. equisimilis
MKKLKRLVISLVTSLLVISSTVPALVYANETNNFAETQKEITTNSEATLTNEDYTKLTSEVKTIYTNLIQYDQTKNKFYVDEDKTEQYYNYDDESIKGVYLMKDSLNDELNNNNSSNYSEIINQKISEIDYVLQGNDINNLIPSNTRVKRSADFSWIQRCLEEAWGYAISLVTLKGIINLFKAGKFEAAAAKLASATAGRIAGMAALFAFVATCGATTVS

SEQ ID NO: 85 - DNA, 663 bp, Streptococcus dysgalactiae
  source 1..663: mol_type = genomic DNA; note = subsp. equisimilis
atgaaaaaattaaaacgtcttgttatctctcttgttacttcattactagtaatttcaagtacagttccagcacttgtttacgctaatgaaacaaataactttgcagaaactcaaaaagaaattacaacaaattcagaagcaacattaaccaatgaagactacactaaattaacttccgaagtaaaaacaatttatacaaatctgattcaatacgaccaaacaaaaaacaaattttacgtcgatgaagacaaaactgaacaatattataactacgatgatgaaagtataaaaggggtttatctcatgaaagatagtttgaacgatgagttaaacaataataactcttcaaactattctgaaataattaatcaaaaaatctctgaaattgactatgtccttcaaggaaacgatataaataatttaattcctagcaataccagagtaaaaagatcagcagatttttcttggattcaaagatgtctagaagaagcatggggatatgctattagtctagttactctaaaaggaataatcaatctatttaaagcaggaaaatttgaagctgctgctgctaaattagcttctgctacagcaggtagaatcgctggaatggctgccttatttgctttcgtagcaacttgcggtgcgacaactgtatcataa

SEQ ID NO: 86 - protein, 57 aa, Enterococcus faecalis
MKQYKVLNEKEMKKPIGGESVFSKIGNAVGPAAYWILKGLGNMSDVNQADRINRKKH

SEQ ID NO: 87 - DNA, 174 bp, Enterococcus faecalis
  source 1..174: mol_type = genomic DNA
atgaagcaatataaagtattgaatgaaaaagaaatgaaaaaacctattgggggagagtcggtttttagtaaaataggtaatgctgtaggtccagctgcttattggattttaaaaggattaggtaatatgagtgatgtaaaccaagctgatagaattaatagaaagaaacattaa

SEQ ID NO: 88 - protein, 44 aa, Enterococcus faecalis
MGAIAKLVAKFGWPIVKKYYKQIMQFIGEGWAINKIIDWIKKHI

SEQ ID NO: 89 - DNA, 135 bp, Enterococcus faecalis
  source 1..135: mol_type = genomic DNA
atgggagcaatcgcaaaattagtagcaaagtttggatggccaattgttaaaaagtattacaaacaaattatgcaatttattggagaaggatgggcaattaacaaaattattgattggatcaaaaaacatatttaa

SEQ ID NO: 90 - protein, 43 aa, Enterococcus faecalis
MGAIAKLVAKFGWPFIKKFYKQIMQFIGQGWTIDQIEKWLKRH

SEQ ID NO: 91 - DNA, 132 bp, Enterococcus faecalis
  source 1..132: mol_type = genomic DNA
atgggagcaatcgcaaaattagtagcaaagtttggatggccatttattaaaaaattctacaaacaaattatgcagtttatcggacaaggatggacaatagatcaaattgaaaaatggttaaaaagacattga

SEQ ID NO: 92 - protein, 74 aa, Enterococcus faecalis
MLNKKLLENGVVNAVTIDELDAQFGGMSKRDCNLMKACCAGQAVTYAIHSLLNRLGGDSSDPAGCNDIVRKYCK

SEQ ID NO: 93 - DNA, 225 bp, Enterococcus faecalis
  source 1..225: mol_type = genomic DNA
atgttaaataaaaaattattagaaaatggtgtagtaaatgctgtaacaattgatgaacttgatgctcaatttggtggaatgagcaaacgtgattgtaacttgatgaaggcgtgttgtgctggacaagcagtaacatatgctattcatagtcttttaaatcgattaggtggagactctagtgatccagctggttgtaatgatattgtaagaaaatattgtaaataa

SEQ ID NO: 94 - protein, 65 aa, Enterococcus faecium
MKHLKILSIKETQLIYGGTTHSGKYYGNGVYCTKNKCTVDWAKATTCIAGMSIGGFLGGAIPGKC

SEQ ID NO: 95 - DNA, 195 bp, Enterococcus faecium
  source 1..195: mol_type = genomic DNA
atgaaacatttaaaaattttgtctattaaagagacacaacttatctatgggggtaccactcatagtggaaaatattatggaaatggagtgtattgcactaaaaataaatgtacggtcgattgggccaaggcaactacttgtattgcaggaatgtctataggtggttttttaggtggagcaattccagggaagtgc

SEQ ID NO: 96 - protein, 105 aa, Enterococcus faecalis
MVKENKFSKIFILMALSFLGLALFSASLQFLPIAHMAKEFGIPAAVAGTVLNVVEAGGWVTTIVSILTAVGSGGLSLLAAAGRESIKAYLKKEIKKKGKRAVIAW

SEQ ID NO: 97 - DNA, 318 bp, Enterococcus faecalis
  source 1..318: mol_type = genomic DNA
atggttaaagaaaataaattttctaagatttttattttaatggctttgagttttttggggttagccttgtttagtgcaagtcttcagtttttgcccattgcacatatggctaaagagttcggtataccagcagcagttgcaggaactgtgcttaatgtagttgaagctggtggatgggtcactactattgtatcaattcttactgctgtaggtagcggaggtctttctttactcgctgcagcaggaagagagtcaattaaagcataccttaagaaagaaattaagaaaaaaggaaaaagagcagttattgcttggtaa

SEQ ID NO: 98 - protein, 71 aa, Enterococcus faecium
MQNVKELSTKEMKQIIGGENDHRMPNELNRPNNLSKGGAKCGAAIAGGLFGIPKGPLAWAAGLANVYSKCN

SEQ ID NO: 99 - DNA, 216 bp, Enterococcus faecium
  source 1..216: mol_type = genomic DNA
atgcaaaatgtaaaagaattaagtacgaaagagatgaaacaaattatcggtggagaaaatgatcacagaatgcctaatgagttaaatagacctaacaacttatctaaaggtggagcaaaatgtggtgctgcaattgctgggggattatttggaatcccaaaaggaccactagcatgggctgctgggttagcaaatgtatactctaaatgcaactaa

SEQ ID NO: 100 - protein, 58 aa, Enterococcus mundtii
MKKLTSKEMAQVVGGKYYGNGVSCNKKGCSVDWGKAIGIIGNNSAANLATGGAAGWKS

SEQ ID NO: 101 - DNA, 177 bp, Enterococcus mundtii
  source 1..177: mol_type = genomic DNA
ttgaagaaattaacatcaaaagaaatggcacaagtagtaggtggaaaatactacggtaatggagtctcatgtaataaaaaagggtgcagtgttgattggggaaaagctattggcattattggaaataattctgctgcgaatttagctactggtggagcagctggttggaaaagttaa

SEQ ID NO: 102 - protein, 44 aa, Enterococcus faecalis
MLAKIKAMIKKFPNPYTLAAKLTTYEINWYKQQYGRYPWERPVA

SEQ ID NO: 103 - DNA, 135 bp, Enterococcus faecalis
  source 1..135: mol_type = genomic DNA
atgttagcaaaaattaaagcgatgattaagaagtttccgaacccttatactttagcagctaagctaacgacttacgaaattaattggtataaacaacaatacggtcgttatccttgggagcgccctgtagcataa

SEQ ID NO: 104 - protein, 71 aa, Enterococcus faecium
MRKKLFSLALIGIFGLVVTNFGTKVDAATRSYGNGVYCNNSKCWVNWGEAKENIAGIVISGWASGLAGMGH

SEQ ID NO: 105 - DNA, 216 bp, Enterococcus faecium
  source 1..216: mol_type = genomic DNA
atgagaaaaaaattatttagtttagctcttattggaatatttgggttagttgtgacaaattttggtacaaaagttgatgcagctacgcgttcatatggtaatggtgtttattgtaataatagtaaatgctgggttaactggggagaagctaaagagaatattgcaggaatcgttattagtggctgggcttctggtttggcaggtatgggacattaa

SEQ ID NO: 106 - protein, 34 aa, Enterococcus faecium
MNFLKNGIAKWMTGAELQAYKKKYGCLPWEKISC

SEQ ID NO: 107 - DNA, 105 bp, Enterococcus faecium
  source 1..105: mol_type = genomic DNA
atgaattttcttaaaaatggtatcgcaaaatggatgaccggtgctgaattgcaagcgtataaaaagaaatatggatgcttgccatgggaaaaaatttcttgttaa

SEQ ID NO: 108 - protein, 76 aa, Enterococcus faecalis
MKKKLVKGLVICGMIGIGFTALGTNVEAATYYGNGVYCNKQKCWVDWSRARSEIIDRGVKAYVNGFTKVLGGIGGR

SEQ ID NO: 109 - DNA, 231 bp, Enterococcus faecalis
  source 1..231: mol_type = genomic DNA
atgaaaaagaaattagttaaaggcttagttatttgtggcatgattgggattggttttacagcattaggaacaaatgtagaagccgccacgtattacggaaatggtgtctattgcaataagcaaaaatgttgggtagattggagtagagcacgttctgaaattatagacagaggcgtaaaagcatacgtcaatggatttacgaaagtgttaggtggtataggtggaagataa

SEQ ID NO: 110 - protein, 60 aa, Enterococcus faecalis
MKKEELVGMAKEDFLNVICENDNKLENSGAKCPWWNLSCHLGNDGKICTYSHECTAGCNA

SEQ ID NO: 111 - DNA, 183 bp, Enterococcus faecalis
  source 1..183: mol_type = genomic DNA
atgaaaaaagaagaattagtaggaatggctaaggaagactttttaaatgttatttgtgaaaatgacaacaaactagaaaatagtggagcaaaatgtccttggtggaatctttcttgtcatttaggcaatgatggtaaaatttgcacttattcacatgaatgtaccgcaggttgtaatgcataa

SEQ ID NO: 112 - protein, 61 aa, Enterococcus faecalis
MTELNKRLQLKRDVSTENSLKKISNTDETHGGVTTSIPCTVMVSAAVCPTLVCSNKCGGRG

SEQ ID NO: 113 - DNA, 186 bp, Enterococcus faecalis
  source 1..186: mol_type = genomic DNA
atgactgaacttaacaaaagattacaattaaaaagagatgtttcaacagaaaatagtttgaaaaaaatttctaatactgatgaaacacatgggggagttactacatcaattccatgtacagtaatggttagtgcggcagtatgtcctacccttgtttgctcgaataaatgtggcggtagaggctag

SEQ ID NO: 114 - protein, 58 aa, Enterococcus faecium
MQNVKEVSVKEMKQIIGGSNDSLWYGVGQFMGKQANCITNHPVKHMIIPGYCLSKILG

SEQ ID NO: 115 - DNA, 177 bp, Enterococcus faecium
  source 1..177: mol_type = genomic DNA
atgcaaaatgtaaaagaagtttctgtaaaagagatgaaacaaattatcggtggttctaatgatagtctttggtatggtgtaggacaatttatgggtaaacaagcaaactgtataacaaaccatcctgttaaacacatgataattcctggatattgtttatcgaaaattttagggtaa

SEQ ID NO: 116 - protein, 55 aa, Enterococcus faecium
MKKYNELSKKELLQIQGGIAPIIVAGLGYLVKDAWDHSDQIISGFKKGWNGGRRK

SEQ ID NO: 117 - DNA, 168 bp, Enterococcus faecium
  source 1..168: mol_type = genomic DNA
atgaaaaaatataatgagttatctaaaaaagaacttctacagattcaaggaggaatagcacctattatagttgctggccttggctatttagtaaaagatgcatgggatcactcagatcaaataatctcaggatttaaaaaaggttggaatggtggacgtagaaaataa

SEQ ID NO: 118 - protein, 343 aa, Enterococcus faecalis
MKNILLSILGVLSIVVSLAFSSYSVNAASNEWSWPLGKPYAGRYEEGQQFGNTAFNRGGTYFHDGFDFGSAIYGNGSVYAVHDGKILYAGWDPVGGGSLGAFIVLQAGNTNVIYQEFSRNVGDIKVSTGQTVKKGQLIGKFTSSHLHLGMTKKEWRSAHSSWNKDDGTWFNPIPILQGGSTPTPPNPGPKNFTTNVRYGLRVLGGSWLPEVTNFNNTNDGFAGYPNRQHDMLYIKVDKGQMKYRVHTAQSGWLPWVSKGDKSDTVNGAAGMPGQAIDGVQLNYITPKGEKLSQAYYRSQTTKRSGWLKVSADNGSIPGLDSYAGIFGEPLDRLQIGISQSNPF

SEQ ID NO: 119 - DNA, 1032 bp, Enterococcus faecalis
  source 1..1032: mol_type = genomic DNA
atgaaaaatattttactttctattctaggggtattatctatcgttgtttctttggcgttttcttcttattctgtcaacgcagcttctaatgagtggtcgtggccactgggcaaaccatatgcgggaagatatgaagaaggacaacaattcgggaacactgcatttaaccgaggaggtacttatttccatgatgggtttgactttggttctgctatttatggaaatggcagtgtgtatgctgtgcatgatggtaaaattttatatgctggttgggatcctgtaggtggaggctcattaggtgcatttattgtactacaagcgggaaacacaaatgtgatttatcaagaatttagccgaaatgttggagatattaaagttagcactggacaaactgttaaaaaaggacagctgataggaaagtttacttctagtcatttacatttaggaatgacaaaaaaagaatggcgttctgctcattcttcttggaataaagatgatggcacttggtttaacccaattcctatacttcaaggaggatctacgcctacgcctccaaatccaggaccaaaaaatttcacaacaaatgttcgttacggattgcgggtcctcggaggttcatggttaccagaagtaaccaactttaacaataccaatgatggtttcgcaggttaccctaatcgtcaacatgatatgctttatataaaggtagataaagggcaaatgaaatatcgtgttcacacggctcaaagtggatggttgccttgggtaagtaaaggggataagagcgatacagtaaatggagcggcaggtatgcctggacaagcaattgatggtgttcagctaaactatataactcctaagggagaaaaattatcacaggcttactatcgttcacaaactacgaaacgatcaggctggttaaaagtaagtgcagataatggttctattcctggactagacagttatgcaggaatctttggagaaccgttggatcgcttgcaaataggtatttcacagtcaaatccattttaa

SEQ ID NO: 120 - protein, 56 aa, Staphylococcus epidermidis
MENKKDLFDLEIKKDNMENNNELEAQSLGPAIKATRQVCPKATRFVTVSCKKSDCQ

SEQ ID NO: 121 - DNA, 171 bp, Staphylococcus epidermidis
  source 1..171: mol_type = genomic DNA
atggaaaacaaaaaagatttatttgatttagaaatcaaaaaagataatatggaaaataataatgaattagaagctcaatctcttggtcctgcaattaaggcaactagacaggtatgtcctaaagcaacacgttttgttacagtttcttgtaaaaaaagtgattgtcaatag

SEQ ID NO: 122 - protein, 51 aa, Staphylococcus epidermidis
MAAFMKLIQFLATKGQKYVSLAWKHKGTILKWINAGQSFEWIYKQIKKLWA

SEQ ID NO: 123 - DNA, 156 bp, Staphylococcus epidermidis
  source 1..156: mol_type = genomic DNA
atggcagcatttatgaagttaattcagttcttagcaactaaaggtcaaaagtatgtttcacttgcatggaaacataaaggtactattttaaaatggattaacgccggtcaaagttttgaatggatttataaacaaatcaaaaaattatgggcataa

SEQ ID NO: 124 - protein, 52 aa, Staphylococcus epidermidis
MEAVKEKNDLFNLDVKVNAKESNDSGAEPRIASKFICTPGCAKTGSFNSYCC

SEQ ID NO: 125 - DNA, 159 bp, Staphylococcus epidermidis
  source 1..159: mol_type = genomic DNA
atggaagcagtaaaagaaaaaaatgatctttttaatcttgatgttaaagttaatgcaaaagaatctaacgattcaggagctgaaccaagaattgctagtaaatttatatgtactcctggatgtgcaaaaacaggtagttttaacagttattgttgttaa

SEQ ID NO: 126 - protein, 55 aa, Staphylococcus epidermidis
MNNSLFDLNLNKGVETQKSDLSPQSASVLKTSIKVSKKYCKGVTLTCGCNITGGK

SEQ ID NO: 127 - DNA, 168 bp, Staphylococcus epidermidis
  source 1..168: mol_type = genomic DNA
atgaataactcattattcgatttaaacctaaacaaaggtgtagaaactcaaaagagtgatttaagtccgcaatctgctagtgtcttgaagacttctattaaagtatctaaaaaatattgtaaaggtgttactttaacatgcggttgcaatattactggtggtaaataa

SEQ ID NO: 128 - protein, 52 aa, Staphylococcus gallinarum
MEAVKEKNELFDLDVKVNAKESNDSGAEPRIASKFLCTPGCAKTGSFNSYCC

SEQ ID NO: 129 - DNA, 159 bp, Staphylococcus gallinarum
  source 1..159: mol_type = genomic DNA
atggaagcagtaaaagagaaaaatgaactttttgatcttgacgttaaagtaaatgcaaaagagtctaatgattcaggcgcagaaccacgaattgctagtaaatttttatgtactcctggatgtgccaaaacaggtagcttcaatagctactgttgttaa

SEQ ID NO: 130 - protein, 63 aa, Lactococcus garvieae
MENNNYTVLSDEELQKIDGGIGGALGNALNGLGTWANMMNGGGFVNQWQVYANKGKINQYRPY

SEQ ID NO: 131 - DNA, 192 bp, Lactococcus garvieae
  source 1..192: mol_type = genomic DNA
atggaaaacaacaattacacagtactttcagatgaagaactacaaaaaattgatggtggaatcggcggggctcttggtaatgctctcaacggattaggtacctgggcaaacatgatgaacggtggaggatttgttaatcagtggcaagtttatgctaataaaggaaaaataaatcaataccgtccgtattaa

SEQ ID NO: 132 - protein, 63 aa, Lactococcus garvieae
MFDLVATGMAAGVAKTIVNAVSAGMDIATALSLFSGAFTAAGGIMALIKKYAQKKLWKQLIAA

SEQ ID NO: 133 - DNA, 192 bp, Lactococcus garvieae
  source 1..192: mol_type = genomic DNA
atgtttgatttagtcgcgactggaatggctgcaggtgtagcaaaaactattgttaatgccgttagtgctggtatggatattgccactgctttatcattgttctcaggagcttttactgcagctgggggaattatggcactcattaaaaaatatgctcaaaagaaattatggaaacagcttattgctgcataa

SEQ ID NO: 134 - protein, 91 aa, Lactobacillus gasseri
MVTKYGRNLGLNKVELFAIWAVLVVALLLTTANIYWIADQFGIHLATGTARKLLDAMASGASLGTAFAAILGVTLPAWALAAAGALGATAA

SEQ ID NO: 135 - DNA, 276 bp, Lactobacillus gasseri
  source 1..276: mol_type = genomic DNA
atggttactaagtacggacgtaatttaggtttgaacaaggtagagttgtttgcaatttgggcggttttagtagttgctcttttattgaccacagcgaacatttattggattgctgatcaattcgggattcatttagcgactggaacagcccgtaagttattagatgcaatggcttctggtgcctcattgggaactgcctttgctgctattttgggcgtgacattacctgcatgggctttggcagctgcaggagcattgggagcgactgcagcctag

SEQ ID NO: 136 - protein, 75 aa, Lactobacillus gasseri
MKNFNTLSFETLANIVGGRNNWAANIGGVGGATVAGWALGNAVCGPACGFVGAHYVPIAWAGVTAATGGFGKIRK

SEQ ID NO: 137 - DNA, 228 bp, Lactobacillus gasseri
  source 1..228: mol_type = genomic DNA
atgaaaaattttaatacattatcatttgaaacattggctaacatagttggtgggagaaataattgggctgctaatataggtggagtaggtggagcgacagtcgctggatgggctcttggaaatgcagtttgcggtcctgcttgtggctttgttggagcacactatgttccaatagcatgggctggcgtaacggcagctactggtggattcggaaagataagaaagtag

SEQ ID NO: 138 - protein, 64 aa, Lactobacillus plantarum
MSKLVKTLTISEISKAQNNGGKPAWCWYTLAMCGAGYDSGTCDYMYSHCFGIKHHSSGSSSYHC

SEQ ID NO: 139 - DNA, 195 bp, Lactobacillus plantarum
  source 1..195: mol_type = genomic DNA
atgagtaaattggttaagacacttactataagtgaaatttctaaggctcaaaacaacggtggaaaacctgcatggtgttggtatactttagcaatgtgtggtgctggttatgattcgggaacctgtgattatatgtattcgcattgttttggtataaagcatcatagtagtggtagtagcagttatcattgttag

SEQ ID NO: 140 - protein, 359 aa, Haloferax mediterranei
MSKDRDGRRTSRRGTLKKIGGFSLGALSFGAVGRTQAATGSSVTTADIAPPGPNGDPKSVQIDDKYTGAEMYGEGDFRVGLGTDLTMYPPVYRESLGNGSGGWEFDFTVCGSTACRFVDSNGDVKEDDKAKEMWWQEINFNDINQDLYSRNDSDWVGSTPADTQPEFDYTEFALARDGVTLALTALNPAMGSLALGATYFLSDMVNWIASQHEDDSSLKRKWDYDGLSGPLYADSSTYLLARDEMTSNSYESFTIDNIAVAFPEFPVRTKYYVTFTAPDDPSTQSISTLEEEGIYRVPATEVAAARPPGSRRSKSAADEMVYVADPKKFIEVEPVKNPSIPDRIYEEIEQKKKQRSRKQ

SEQ ID NO: 141 - DNA, 1080 bp, Haloferax mediterranei
  source 1..1080: mol_type = genomic DNA
atgtcgaaagacagagatgggagaaggacaagtcggcgaggcacgttaaagaaaatcggcggtttcagtctcggagcgcttagtttcggggcagtcggacgaactcaagcggcgaccggctcatcggttacgaccgctgatatcgcacctcccggaccgaacggagacccgaagagtgttcagatagatgataaatacaccggagccgagatgtacggcgagggtgacttcagagtcggtctcggaactgacctgacgatgtatccgcccgtgtaccgtgagagtcttggaaatggaagcgggggttgggaattcgacttcaccgtttgtgggtccactgcctgtcgatttgtggacagtaacggtgacgtcaaagaggacgacaaggcgaaagaaatgtggtggcaggaaattaacttcaacgacataaatcaggatttatacagtcggaacgattccgactgggtcgggtcgacccctgccgatacccaaccggagttcgattacaccgactttgcgctcgctcgggacggagtgacgctcgctctcacggcactcaaccccgcaatggggagtcttgcactcggtgccacgtacttcctcagcgacatggtgaactggattgcgagccagcacgaagacgacagttcgctcaagagaaaatgggattacgacgggctaagtgggccgttgtacgccgattcgtcgacgtacctactggcacgcgacgagatgacttcgaactcgtacgaatcattcacgatcgataacatcgccgttgccttcccagagttccccgtccggaccaagtactacgtcacattcactgcgccggatgacccgtcaacgcagtcgatatctacgctcgaagaggagggaatctaccgagtgcccgctacggaagtggctgcggccagaccaccggggtcccgacgttccaaatcggcagccgacgagatggtgtacgttgccgatccgaagaagttcatagaggtcgagccggtgaagaacccaagtatcccggaccgaatctacgaggagatagagcaaaaaaagaaacaacggagtaggaaacagtag

SEQ ID NO: 142 - protein, 311 aa, Haloarchaeon S8a
MSDKDSINRRNVLRKIGGIGVASAVGFSGLASGESLSDDEKQDVIDTIYKSQRVEQIKKKFGGVNIEPKKVQSVTTNQSGDLVTAKLSVSDGDLVYSSVKDTTVIVQFDRSASEIGESWPKNTEAFIKSTSSGVDLLRTATDEEIKDVTEGVNTSEIESADAVNIFIDPESQTYYMEKYDFNNKVLEMFELATGGTSSGKISPTREDQNHEYNVREHKVFNSEKQNIQLQSDCNINSNTAADVILCFNQVGSCALCSPTLVGGPVPTVACLLVVCFGTPNAVSAILEEVDNSCFNLIKDVISCWDEWTSFW

SEQ ID NO: 143 - DNA, 936 bp, Haloarchaeon S8a
  source 1..936: mol_type = genomic DNA
atgtcggataaagacagcattaacagaagaaatgtattaagaaaaattggcggtatcggtgtggcttcagctgtcggattttctggtttggcaagcggggaaagtcttagcgatgatgagaaacaagatgttattgacacaatttacaaatcacaaagagttgaacagataaagaaaaagttcggaggagtgaatattgagccgaaaaaggttcaatctgtaacgaccaatcagagcggagatcttgttacggcgaagctgtcggttagtgatggggatttggtatattcgagtgtcaaagatacaactgtaatagttcagttcgatagatcggcttctgaaattggtgaaagttggcccaagaatactgaggcattcatcaaatcgacgtcctctggggtcgatcttctacgtacagcaactgatgaagaaataaaggacgttactgagggagtcaacacatctgaaattgaatctgcggatgctgttaacatatttattgatcctgaatcacagacatactatatggagaaatatgactttaataataaggtacttgagatgtttgaattagcgacaggtgggacaagtagtggtaaaatctcccccacacgtgaagaccagaatcacgaatataatgttagggaacataaagtatttaactcagaaaaacagaatatacaacttcagagtgactgtaatataaacagtaacaccgctgctgatgttattctatgcttcaaccaggttggttcttgtgcactctgctccccgactttagtcggaggtccagtccctacagttgcatgtctcttagtcgtctgtttcggcactccaaatgctgtgtccgcgatacttgaagaagtcgataattcttgctttaacttgatcaaggatgtaatttcgtgttgggatgaatggactagcttctggtga

SEQ ID NO: 144 - protein, 333 aa, Lactobacillus helveticus
MKHLNETTNVRILSQFDMDTGYQAVVQKGNVGSKYVYGLQLRKGATTILRGYRGSKINNPILELSGQAGGHTQTWEFAGDRKDINGEERAGQWFIGVKPSKIEGSKIIWAKQIARVDLRNQMGPHYSNTDFPRLSYLNRAGSNPFAGNKMTHAEAAVSPDYTKFLIATVENNCIGHFTIYNLDTINEKLDEKGNSEDVNLETVKYEDSFIIDNLYGDDNNSIVNSIQGYDLDNDGNIYISSQKAPDFDGSYYAHHKQIVKIPYYARSKESEDQWRAVNLSEFGGLDIPGKHSEVESIQIIGENHCYLTVAYHSKNKAGENKTTLNEIYELSWN

SEQ ID NO: 145 - DNA, 1002 bp, Lactobacillus helveticus
  source 1..1002: mol_type = genomic DNA
atgaagcatttaaatgaaacaactaatgttagaattttaagtcaatttgatatggatactggctatcaagcagtagttcaaaaaggcaatgtaggttcaaaatatgtatatggattacaacttcgcaaaggtgctactactatcttgcgtggttaccgtggaagtaaaattaataaccctattcttgaattatctggtcaagcaggtggtcacacacagacatgggaatttgctggtgatcgtaaagacattaatggtgaagaaagagcaggtcaatggtttataggtgttaaaccatcgaaaattgaaggaagcaaaattatttgggcaaagcaaattgcaagagttgatcttagaaatcaaatgggacctcattattcaaatactgactttcctcgattatcctacttgaatcgcgccggttctaatccatttgctggtaataagatgacgcatgccgaagccgcagtatcacctgattatactaagtttttaattgctactgttgaaaataactgtattggtcattttactatatacaatttagatacaattaatgaaaaacttgatgaaaagggaaatagtgaagatgttaatctcgaaactgttaaatacgaagatagttttatcattgataatttatatggtgatgataataattctattgtaaattcaattcaagggtatgatttggataatgatggaaatatttatatttccagtcaaaaagcgccagattttgatggctcttattatgcacatcataagcagattgttaagattccatattatgctcggtctaaagaaagcgaagaccaatggagagctgtaaatttaagcgaattcggtggcttggatattccaggtaaacatagtgaagttgaaagcatccaaattattggtgagaatcattgttacttaactgttgcatatcattctaaaaataaagcgggtgaaaataaaactactttgaatgagatttatgaattatcttggaattag

SEQ ID NO: 146 - protein, 74 aa, Enterococcus hirae; identical to SEQ ID NO: 20

SEQ ID NO: 147 - DNA, 225 bp, Enterococcus hirae; identical to SEQ ID NO: 21
  source 1..225: mol_type = genomic DNA

SEQ ID NO: 148 - protein, 75 aa, Lactobacillus johnsonii
MKQFNYLSHKDLAVVVGGRNNWQTNVGGAVGSAMIGATVGGTICGPACAVAGAHYLPILWTAVTAATGGFGKIRK

SEQ ID NO: 149 - DNA, 228 bp, Lactobacillus johnsonii
  source 1..228: mol_type = genomic DNA
atgaaacaatttaattatttatcacataaagatttagcagtcgttgttggtggaagaaataattggcaaacaaatgtgggaggagcagtgggatcagctatgattggggctacagttggtggtacaatttgtggacctgcatgtgctgtagctggtgcccattatcttcctattttatggacagcggttacagctgcaacaggtggttttggcaagataagaaagtag

SEQ ID NO: 150 - protein, 62 aa, Lactobacillus johnsonii
MKLNDKELSKIVGGNRWGDTVLSAASGAGTGIKACKSFGPWGMAICGVGGAAIGGYFGYTHN

SEQ ID NO: 151 - DNA, 189 bp, Lactobacillus johnsonii
  source 1..189: mol_type = genomic DNA
atgaaattaaatgacaaagaattatcaaagattgttggtggaaatcgatggggagatactgttttatcagctgctagtggcgcaggaactggtattaaagcatgtaaaagttttggcccatggggaatggcaatttgtggtgtaggaggtgcagcaataggaggttattttggctatactcataattaa

SEQ ID NO: 152 - protein, 59 aa, Lactococcus lactis
  source 1..59: note = subsp. lactis
MNKNEIETQPVTWLEEVSDQNFDEDVFGACSTNTFSLSDYWGNNGAWCTLTHECMAWCK

SEQ ID NO: 153 - DNA, 180 bp, Lactococcus lactis
  source 1..180: mol_type = genomic DNA; note = subsp. lactis
atgaacaaaaatgaaattgaaacacaaccagttacatggttggaagaagtatctgatcaaaattttgatgaagatgtatttggtgcgtgtagtactaacacattctcgctcagtgattactggggaaataacggggcttggtgtacactcactcatgaatgtatggcttggtgtaaataa

SEQ ID NO: 154 - protein, 65 aa, Lactococcus lactis
  source 1..65: note = subsp. lactis
MKEKNMKKNDTIELQLGKYLEDDMIELAEGDESHGGTTPATPAISILSAYISTNTCPTTKCTRAC

SEQ ID NO: 155 - DNA, 198 bp, Lactococcus lactis
  source 1..198: mol_type = genomic DNA; note = subsp. lactis
atgaaagaaaaaaatatgaaaaagaatgacactattgaattacaattgggaaaataccttgaagatgatatgattgaattagctgaaggggatgagtctcatggaggaacaacaccagcaactcctgcaatctctattctcagtgcatatattagtaccaatacttgtccaacaacaaaatgtacacgtgcttgttaa

SEQ ID NO: 156 - protein, 51 aa, Lactococcus lactis
  source 1..51: note = subsp. lactis
MKEQNSFNLLQEVTESELDLILGAKGGSGVIHTISHECNMNSWQFVFTCCS

SEQ ID NO: 157 - DNA, 156 bp, Lactococcus lactis
  source 1..156: mol_type = genomic DNA; note = subsp. lactis
atgaaagaacaaaactcttttaatcttcttcaagaagtgacagaaagtgaattggaccttattttaggtgcaaaaggcggcagtggagttattcatacaatttctcatgaatgtaatatgaatagctggcaatttgtatttacttgctgctcttaa

SEQ ID NO: 158 - protein, 53 aa, Lactococcus lactis
MAGFLKVVQLLAKYGSKAVQWAWANKGKILDWLNAGQAIDWVVSKIKQILGIK

SEQ ID NO: 159 - DNA, 162 bp, Lactococcus lactis
  source 1..162: mol_type = genomic DNA
atggcagggtttttaaaagtagttcaattactagctaaatatggttctaaagctgtacaatgggcttgggcaaacaagggtaagattttagattggcttaatgcaggtcaggctattgattgggtagtttcgaaaattaagcaaattttaggtattaagtaa

SEQ ID NO: 160 - protein, 53 aa, Lactococcus lactis
MAGFLKVVQILAKYGSKAVQWAWANKGKILDWINAGQAIDWVVEKIKQILGIK

SEQ ID NO: 161 - DNA, 162 bp, Lactococcus lactis
  source 1..162: mol_type = genomic DNA
atggcagggtttttaaaagtagtccaaattttggctaagtatggttctaaagccgtacaatgggcatgggcaaataaaggaaaaatcttagattggattaatgcaggtcaagctattgactgggtagttgaaaagattaagcaaattttgggtattaaataa

SEQ ID NO: 162 - protein, 65 aa, Lactobacillus amylovorus
MKQLNSEQLQNIIGGNRWTNAYSAALGCAVPGVKYGKKLGGVWGAVIGGVGGAAVCGLAGYVRKG

SEQ ID NO: 163 - DNA, 198 bp, Lactobacillus amylovorus
  source 1..198: mol_type = genomic DNA
atgaaacaattgaattcagaacaattacaaaatattatcggtggaaatagatggactaatgcatacagcgcagctttgggatgcgctgtccctggagttaaatatggaaaaaaacttggtggcgtatggggtgctgtaattggtggcgtaggcggtgcagcagtctgtggcttggcgggttatgttcgtaaaggctaa

SEQ ID NO: 164 - protein, 68 aa, Lactobacillus sakei
  source 1..68: note = L45
MKTEKKVLDELSLHASAKMGARDVESSMNADSTPVLASVAVSMELLPTASVLYSDVAGCFKYSAKHHC

SEQ ID NO: 165 - DNA, 207 bp, Lactobacillus sakei
  source 1..207: mol_type = genomic DNA; note = L45
atgaaaacagaaaaaaaggttttagatgaactgagcttacacgcttctgcaaaaatgggagcacgtgatgttgaatccagcatgaatgcagactcaacaccagttttagcatcagtcgctgtatccatggaattattgccaactgcgtctgttctttattcggatgttgcaggttgcttcaaatattctgcaaaacatcattgttag

SEQ ID NO: 166 - protein, 91 aa, Lactococcus lactis
  source 1..91: note = subsp. lactis
MKTKSLVLALSAVTLFSAGGIVAQAEGTWQHGYGVSSAYSNYHHGSKTHSATVVNNNTGRQGKDTQRAGVWAKATVGRNLTEKASFYYNFW

SEQ ID NO: 167 - DNA, 276 bp, Lactococcus lactis
  source 1..276: mol_type = genomic DNA; note = subsp. lactis
atgaaaaccaagtctctcgtattggcattatctgcggttacgttattctctgccggaggaattgtagctcaagctgaaggaacatggcaacatggatatggtgttagttcggcatattcaaattatcatcatggtagcaaaactcattcagccacagttgtaaataataatactggccgacaaggtaaggatacacaacgtgccggtgtttgggcaaaagctactgttggacgtaacttaactgaaaaagcttcattttattataacttttggtaa

SEQ ID NO: 168 - protein, 75 aa, Lactococcus lactis
  source 1..75: note = subsp. cremoris
MKNQLNFNIVSDEELSEANGGKLTFIQSTAAGDLYYNTNTHKYVYQQTQNAFGAAANTIVNGWMGGAAGGFGLHH

SEQ ID NO: 169 - DNA, 228 bp, Lactococcus lactis
  source 1..228: mol_type = genomic DNA; note = subsp. cremoris
atgaaaaatcaattaaattttaatattgtttcagatgaagaactttcagaagctaacggaggaaaattaacatttattcaatcgacagcggctggagatttatattacaatactaatacacacaaatatgtttaccaacaaactcaaaacgcttttggggctgctgctaataccattgttaatggatggatgggtggcgctgctggaggtttcgggttgcaccattga

SEQ ID NO: 170 - protein, 68 aa, Lactococcus lactis
  source 1..68: note = subsp. cremoris
MKNQLNFNIVSDEELAEVNGGSLQYVMSAGPYTWYKDTRTGKTICKQTIDTASYTFGVMAEGWGKTFH

SEQ ID NO: 171 - DNA, 207 bp, Lactococcus lactis
  source 1..207: mol_type = genomic DNA; note = subsp. cremoris
atgaaaaatcaattaaattttaatattgtttctgatgaagaacttgcagaagttaatggaggaagcttgcagtatgttatgagtgctggaccatatacttggtataaagatactagaacaggaaaaacaatatgtaaacagacaattgacacagcaagttatacatttggtgtaatggcagaaggatggggaaaaacattccactaa

SEQ ID NO: 172 - protein, 63 aa, Lactococcus sp.
  source 1..63: note = QU 12
MKLIDHLGAPRWAVDTILGAIAVGNLASWVLALVPGPGWAVKAGLATAAAIVKHQGKAAAAAW

SEQ ID NO: 173 - DNA, 192 bp, Lactococcus sp.
  source 1..192: mol_type = genomic DNA; strain = QU 12
atgaaattaattgatcatttaggtgctccaagatgggccgttgatactattttaggtgcaatcgcagttgggaacttagcaagttgggttctagcgcttgtccctggtccagggtgggcagtaaaagctggtttagcaactgctgctgccatcgttaaacatcaaggtaaagctgccgctgctgcttggtaa

SEQ ID NO: 174 - protein, 50 aa, Brevibacillus sp.
  source 1..50: note = GI-9
MACQCPDAISGWTHTDYQCHGLENKMYRHVYAICMNGTQVYCRTEWGSSC

SEQ ID NO: 175 - DNA, 153 bp, Brevibacillus sp.
  source 1..153: mol_type = genomic DNA; strain = GI-9
atggcttgccaatgtccagatgcgatctcaggttggacgcatacagattaccagtgtcacggtttggagaataaaatgtatagacatgtttatgcaatttgcatgaacggtactcaagtatattgcagaacagagtggggtagcagctgctag

SEQ ID NO: 176 - protein, 57 aa, Leuconostoc pseudomesenteroides
MNKEYNSISNFKKITNKDLQNINGGFIGRAIGDFVYFGAKGLRESGKLLNYYYKHKH

SEQ ID NO: 177 - DNA, 174 bp, Leuconostoc pseudomesenteroides
  source 1..174: mol_type = genomic DNA
atgaataaagaatataatagcattagcaattttaaaaaaattactaataaagacttgcaaaacataaatggtggatttattggtagggcaataggtgactttgtgtactttggagcgaagggactaagagaatctggtaaactacttaattattactataagcataagcattga

SEQ ID NO: 178 - protein, 53 aa, Leuconostoc pseudomesenteroides
MKNQLMSFEVISEKELSTVQGGKGLGKLIGIDWLLGQAKDAVKQYKKDYKRWH

SEQ ID NO: 179 - DNA, 162 bp, Leuconostoc pseudomesenteroides
  source 1..162: mol_type = genomic DNA
atgaaaaatcagttaatgtctttcgaagtgatatcagaaaaagaattgtccacggtacaaggtggcaaaggcttaggtaaactcataggaattgattggcttttgggtcaagctaaggacgctgttaaacagtacaagaaggattacaaacgttggcactaa

SEQ ID NO: 180 - protein, 61 aa, Leuconostoc gelidum
MMNMKPTESYEQLDNSALEQVVGGKYYGNGVHCTKSGCSVNWGEAFSAGVHRLANGGNGFW

SEQ ID NO: 181 - DNA, 186 bp, Leuconostoc gelidum
  source 1..186: mol_type = genomic DNA
atgatgaacatgaaacctacggaaagctatgagcaattggataatagtgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 182 - protein, 61 aa, Leuconostoc carnosum
MNNMKSADNYQQLDNNALEQVVGGKYYGNGVHCTKSGCSVNWGEAFSAGVHRLANGGNGFW

SEQ ID NO: 183 - DNA, 186 bp, Leuconostoc carnosum
  source 1..186: mol_type = genomic DNA
atgaataacatgaaatctgcggataattatcagcaattggataataatgctctcgaacaagtcgtaggaggtaagtattatggtaacggagttcattgcacaaaaagtggttgttctgtaaactggggagaagccttttcagctggagtacatcgtttagcaaatggtggaaatggtttctggtaa

SEQ ID NO: 184 - protein, 63 aa, Leuconostoc mesenteroides
MFLVNQLGISKSLANTILGAIAVGNLASWLLALVPGPGWATKAALATAETIVKHEGKAAAIAW

SEQ ID NO: 185 - DNA, 192 bp, Leuconostoc mesenteroides
  source 1..192: mol_type = genomic DNA
atgttcttggtaaatcagttagggatttcaaaatcgttagctaatactattcttggtgcaattgctgttggtaatttggccagttggttattagctttggttcctggtccgggttgggcaacaaaagcagcacttgcgacagctgaaacaattgtgaagcatgaaggaaaagcagctgctattgcgtggtaa

SEQ ID NO: 186 - protein, 74 aa, Bacillus licheniformis
MSKKEMILSWKNPMYRTESSYHPAGNILKELQEEEQHSIAGGTITLSTCAILSKPLGNNGYLCTVTKECMPSCN

SEQ ID NO: 187 - DNA, 225 bp, Bacillus licheniformis
  source 1..225: mol_type = genomic DNA
atgtcaaaaaaggaaatgattctttcatggaaaaatcctatgtatcgcactgaatcttcttatcatccagcagggaacatccttaaagaactccaggaagaggaacagcacagcatcgccggaggcacaatcacgctcagcacttgtgccatcttgagcaagccgttaggaaataacggatacctgtgtacagtgacaaaagaatgcatgccaagctgtaactaa

SEQ ID NO: 188 - protein, 266 aa, Brevibacterium linens
MNNLYRELAPIPGPAWAEIEEEARRTFKRNIAGRRIVDVAGPTGFETSAVTTGHIRDVQSETSGLQVKQRIVQEYIELRTPFTVTRQAIDDVARGSGDSDWQPVKDAATTIAMAEDRAILHGLDAAGIGGIVPGSSNAAVAIPDAVEDFADAVAQALSVLRTVGVDGPYSLLLSSAEYTKVSESTDHGYPIREHLSRQLGAGEIIWAPALEGALLVSTRGGDYELHLGQDLSIGYYSHDSETVELYLQETFGFLALTDESSVPLSL

SEQ ID NO: 189 - DNA, 801 bp, Brevibacterium linens
  source 1..801: mol_type = genomic DNA
gtgaataacctctatcgcgagcttgcccccatccccggcccggcctgggcggagatcgaggaggaggctcgacggacattcaaacgcaatatcgccggccgccggatcgtcgatgtcgcagggcccacgggcttcgagacctccgcggtgaccactggccacatccgagacgtccagtcggagacgagcggactgcaggttaagcagcgcatcgtgcaggaatacatcgagctgcggaccccattcaccgtgactcggcaggccatcgatgacgtggcccgcgggtccggtgactcggactggcagcccgtcaaggatgcggccacgacgatcgcgatggctgaagatcgggccattctccacgggctcgatgcggccgggatcggcggaatcgttcccggcagctcgaatgccgcagtggccatccccgacgccgtcgaggacttcgcggacgccgtcgcccaggcgctgagtgtgctgcgcacggtgggagtcgacgggccctacagcctgttgctctcctccgcggagtacaccaaggtctccgagtccaccgaccacggctacccgatccgcgagcacctctcccggcagctcggcgccggagagatcatctgggcgcccgcgctcgaaggggcgctgctcgtctccacgcgcgggggtgactacgagctccacctcggccaggacctgtcgatcggttactacagccacgacagcgagaccgtcgaactctatctgcaggagaccttcggattcctcgcgctgaccgacgaatccagtgtgcctttgagcctctga

SEQ ID NO: 190 - protein, 71 aa, Listeria innocua
MKKAALKFIIVIAILGFSFSFFSIQSEAKSYGNGVQCNKKKCWVDWGSAISTIGNNSAANWATGGAAGWKS

SEQ ID NO: 191 - DNA, 216 bp, Listeria innocua
  source 1..216: mol_type = genomic DNA
ttgaagaaggcagcgttaaaatttattattgttattgctattctaggtttcagtttttctttctttagcatacaatctgaagctaaatcttatggaaatggagttcagtgtaataagaaaaaatgttgggtagattggggtagtgctataagtactattggaaataattctgcagcgaattgggctacaggtggagcagctggttggaaaagctga

SEQ ID NO: 192 - protein, 68 aa, Bacillus sp.
MSQEAIIRSWKDPFSRENSTQNPAGNPFSELKEAQMDKLVGAGDMEAACTFTLPGGGGVCTLTSECIC

SEQ ID NO: 193 - DNA, 207 bp, Bacillus sp.
  source 1..207: mol_type = genomic DNA
atgagtcaagaagctatcattcgttcatggaaagatcctttttcccgtgaaaattctacacaaaatccagctggtaacccattcagtgagctgaaagaagcacaaatggataagttagtaggtgcgggagacatggaagcagcatgtacttttacattgcctggtggcggcggtgtttgtactctaacttctgaatgtatttgttaa

SEQ ID NO: 194 - protein, 61 aa, Leuconostoc mesenteroides
MTNMKSVEAYQQLDNQNLKKVVGGKYYGNGVHCTKSGCSVNWGEAASAGIHRLANGGNGFW

SEQ ID NO: 195 - DNA, 186 bp, Leuconostoc mesenteroides
  source 1..186: mol_type = genomic DNA
atgacgaatatgaagtctgtggaagcatatcagcaattagataaccagaatctcaagaaagttgttggtggaaagtattatgggaatggtgttcactgtacaaaaagtggatgctctgttaactggggagaagctgcctcagctggcatacatcgtttggccaatggtggaaatggattttggtaa

SEQ ID NO: 196 - protein, 68 aa, Clavibacter michiganensis
  source 1..68: note = subsp. michiganensis
MNDILETETPVMVSPRWDMLLDAGEDTSPSVQTQIDAEFRRVVSPYMSSSGWLCTLTIECGTIICACR

SEQ ID NO: 197 - DNA, 207 bp, Clavibacter michiganensis
  source 1..207: mol_type = genomic DNA; note = subsp. michiganensis
atgaacgacatcctcgagacggagacccccgtcatggtcagcccccggtgggacatgctgctcgacgcgggcgaggacaccagcccgtccgtccagacccagatcgacgcggagttccgtcgcgtcgtgagcccgtacatgtccagcagcggctggctctgcacgctcaccatcgaatgtggcaccatcatctgcgcgtgtcgctga

SEQ ID NO: 198 - protein, 69 aa, Escherichia coli
MELKASEFGVVLSVDALKLSRQSPLGVGIGGGGGGGGGGSCGGQGGGCGGCSNGCSGGNGGSGGSGSHI

SEQ ID NO: 199 - DNA, 207 bp, Escherichia coli
  source 1..207: mol_type = genomic DNA
atggaattaaaagcgagtgaatttggtgtagttttgtccgttgatgctcttaaattatcacgccagtctccattaggtgttggcattggtggtggtggcggcggcggcggcggcggtagctgcggtggtcaaggtggcggttgtggtggttgcagcaacggttgtagtggtggaaacggtggcagcggcggaagtggttcacatatc

SEQ ID NO: 200 - protein, 7 aa, Escherichia coli
MRTGNAN

SEQ ID NO: 201 - DNA, 24 bp, Escherichia coli
  source 1..24: mol_type = genomic DNA
atgcgtactggtaatgcaaactaa

SEQ ID NO: 202 - protein, 99 aa, Klebsiella pneumoniae
MREISQKDLNLAFGAGETDPNTQLLNDLGNNMAWGAALGAPGGLGSAALGAAGGALQTVGQGLIDHGPVNVPIPVLIGPSWNGSGSGYNSATSSSGSGS

SEQ ID NO: 203 - DNA, 300 bp, Klebsiella pneumoniae
  source 1..300: mol_type = genomic DNA
atgagagaaattagtcaaaaggacttaaatcttgcttttggtgcaggagagaccgatccaaatactcaacttctaaacgaccttggaaataatatggcatggggtgctgctcttggcgctcctggcggattaggatcagcagctttgggggccgcgggaggtgcattacaaactgtagggcaaggattaattgaccatggtcctgtaaatgtccccatccctgtactcatcgggccaagctggaatggtagcggtagtggttataacagcgcaacatccagttccggtagtggtagttaa

SEQ ID NO: 204 - protein, 75 aa, Escherichia coli
MREITESQLRYISGAGGAPATSANAAGAAAIVGALAGIPGGPLGVVVGAVSAGLTTAIGSTVGSGSASSSAGGGS

SEQ ID NO: 205 - DNA, 228 bp, Escherichia coli
  source 1..228: mol_type = genomic DNA
atgcgagaaataacagaatcacagttaagatatatttccggggcgggaggtgcgccagcgacttcagctaatgccgcaggtgctgcagctattgttggagctctcgccggaatacctggtggtccacttggggttgtagttggagccgtatctgccggtttgacaacagcaattggctcgaccgtgggaagtggtagtgccagttcttctgctggtggcggtagctaa

SEQ ID NO: 206 - protein, 58 aa, Escherichia coli
MIKHFHFNKLSSGKKNNVPSPAKGVIQIKKSASQLTKGGAGHVPEYFVGIGTPISFYG

SEQ ID NO: 207 - DNA, 177 bp, Escherichia coli
  source 1..177: mol_type = genomic DNA
atgattaagcattttcattttaataaactgtcttctggtaaaaaaaataatgttccatctcctgcaaagggggttatacaaataaaaaaatcagcatcgcaactcacaaaaggtggtgcaggacatgtgcctgagtattttgtggggattggtacacctatatctttctatggctga

SEQ ID NO: 208 - protein, 90 aa, Escherichia coli
MYMRELDREELNCVGGAGDPLADPNSQIVRQIMSNAAWGPPLVPERFRGMAVGAAGGVTQTVLQGAAAHMPVNVPIPKVPMGPSWNGSKG

SEQ ID NO: 209 - DNA, 273 bp, Escherichia coli
  source 1..273: mol_type = genomic DNA
atgtatatgagagagttagatagagaggaattaaattgcgttggtggggctggagatccgcttgcagatcctaattcccaaattgtaagacagataatgtctaatgcggcatggggcccgcctttggtgccagagcggtttaggggaatggctgttggagccgcaggtggggttacgcagacagttcttcaaggagcagcagctcatatgccggttaatgtccctatacctaaagttccgatgggaccctcatggaacggaagtaaaggataa

SEQ ID NO: 210 - protein, 50 aa, Enterococcus mundtii
MSQVVGGKYYGNGVSCNKKGCSVDWGKAIGIIGNNSAANLATGGAAGWKS

SEQ ID NO: 211 - DNA, 153 bp, Enterococcus mundtii
  source 1..153: mol_type = genomic DNA
atgtcacaggtagtaggtggaaaatactacggtaatggagtctcatgtaataaaaaagggtgcagtgttgattggggaaaagcgattggcattattggaaataattctgctgcgaatttagctactggtggagcagctggttggaaaagttaa

SEQ ID NO: 212 - protein, 58 aa, Enterococcus mundtii
MKKLTSKEMAQVVGGKYYGNGLSCNKKGCSVDWGKAIGIIGNNSAANLATGGAAGWKS

SEQ ID NO: 213 - DNA, 177 bp, Enterococcus mundtii
  source 1..177: mol_type = genomic DNA
ttgaagaaattaacatcaaaagaaatggcacaagtagtaggtgggaaatactacggtaatggattatcatgtaataaaaaagggtgcagtgttgattggggaaaagctattggcattattggaaataattctgctgcgaatttagctactggtggagcagctggttggaaaagttaa

SEQ ID NO: 214 - protein, 63 aa, Streptococcus mutans
MSNTQLLEVLGTETFDVQEDLFAFDTTDTTIVASNDDPDTRFKSWSLCTPGCARTGSFNSYCC

SEQ ID NO: 215 - DNA, 192 bp, Streptococcus mutans
  source 1..192: mol_type = genomic DNA
atgtcaaacacacaattattagaagtccttggtactgaaacttttgatgttcaagaagatctctttgcttttgatacaacagatactactattgtggcaagcaacgacgatccagatactcgtttcaaaagttggagcctttgtacgcctggttgtgcaaggacaggtagtttcaatagttactgttgctga

SEQ ID NO: 216 - protein, 53 aa, Streptococcus mutans
MNKLNSNAVVSLNEVSDSELDTILGGNRWWQGVVPTVSYECRMNSWQHVFTCC

SEQ ID NO: 217 - DNA, 162 bp, Streptococcus mutans
  source 1..162: mol_type = genomic DNA
atgaacaagttaaacagtaacgcagtagtttctttgaatgaagtttcagattctgaattggatactattttgggtggtaatcgttggtggcaaggtgttgtgccaacggtctcatatgagtgtcgcatgaattcatggcaacatgttttcacttgctgttaa

SEQ ID NO: 218 - protein, 57 aa, Lactococcus lactis
  source 1..57: note = subsp. lactis
MSTKDFNLDLVSVSKKDSGASPRITSISLCTPGCKTGALMGCNMKTATCHCSIHVSK

SEQ ID NO: 219 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtcattgtagtattcacgtaagcaaataa

SEQ ID NO: 220 - protein, 57 aa, Lactococcus lactis
MSTKDFNLDLVSVSKKDSGASPRITSISLCTPGCKTGALMGCNMKTATCNCSVHVSK

SEQ ID NO: 221 - DNA, 171 bp, Lactococcus lactis
  source 1..171: mol_type = genomic DNA
atgagtacaaaagatttcaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagcgttcacgtaagcaaa

SEQ ID NO: 222 - protein, 57 aa, Lactococcus lactis
MSTKDFNLDLVSVSKTDSGASTRITSISLCTPGCKTGVLMGCNLKTATCNCSVHVSK

SEQ ID NO: 223 - DNA, 174 bp, Lactococcus lactis
  source 1..174: mol_type = genomic DNA
atgagtacaaaagatttcaacttagatttggtatctgtttcaaaaacagattctggcgcttcaacacgtattaccagcatttcgctttgtacaccaggttgtaaaacaggtgttctgatgggatgtaacctgaaaacagcaacttgtaattgtagcgttcacgtaagcaaataa

SEQ ID NO: 224 - protein, 55 aa, Streptococcus uberis
MNNEDFNLDLIKISKENNSGASPRITSKSLCTPGCKTGILMTCPLKTATCGCHFG

SEQ ID NO: 225 - DNA, 168 bp, Streptococcus uberis
  source 1..168: mol_type = genomic DNA
atgaacaatgaagattttaatttggatctcatcaaaatctcaaaggaaaacaactcaggagcttcacctcgaataactagtaaatcattatgtactcctggatgtaagacgggtattttgatgacttgtccactaaaaactgcaacctgtggttgtcattttggataa

SEQ ID NO: 226 - protein, 57 aa, Lactococcus lactis
  source 1..57: note = subsp. lactis
MSTKDFNLDLVSVSKKDSGASPRITSISLCTPGCKTGALMGCNMKTATCNCSIHVSK

SEQ ID NO: 227 - DNA, 174 bp, Lactococcus lactis
  source 1..174: mol_type = genomic DNA; note = subsp. lactis
atgagtacaaaagattttaacttggatttggtatctgtttcgaagaaagattcaggtgcatcaccacgcattacaagtatttcgctatgtacacccggttgtaaaacaggagctctgatgggttgtaacatgaaaacagcaacttgtaattgtagtattcacgtaagcaaataa

SEQ ID NO: 228 - protein, 57 aa, Staphylococcus warneri
MENSKVMKDIEVANLLEEVQEDELNEVLGAKKKSGVIPTVSHDCHMNSFQFVFTCCS

SEQ ID NO: 229 - DNA, 174 bp, Staphylococcus warneri
  source 1..174: mol_type = genomic DNA
atggaaaattctaaagttatgaaggacattgaagtagcaaatttattagaagaggttcaagaagatgaattgaatgaagtcttaggagctaagaaaaagtcaggagtaatcccaactgtgtcacacgattgccatatgaattctttccaatttgtatttacttgttgttcataa

SEQ ID NO: 230 - protein, 58 aa, Paenibacillus polymyxa
MAENLFDLDIQVNKSQGSVEPQVLSIVACSSGCGSGKTAASCVETCGNRCFTNVGSLC

SEQ ID NO: 231 - DNA, 177 bp, Paenibacillus polymyxa
  source 1..177: mol_type = genomic DNA
atggctgaaaacttatttgatctggacattcaagtaaacaaatctcaaggttctgtagagcctcaggttctgagcattgttgcatgttctagcggatgtggtagcggtaaaacagctgccagttgtgttgaaacttgtggcaaccggtgctttactaacgttggttcactctgctaa

SEQ ID NO: 232 - protein, 62 aa, Pediococcus acidilactici
MKKIEKLTEKEMANIIGGKYYGNGVTCGKHSCSVDWGKATTCIINNGAMAWATGGHQGNHKC

SEQ ID NO: 233 - DNA, 189 bp, Pediococcus acidilactici
  source 1..189: mol_type = genomic DNA
atgaaaaaaattgaaaaattaactgaaaaagaaatggccaatatcattggtggtaaatactacggtaatggggttacttgtggcaaacattcctgctctgttgactggggtaaggctaccacttgcataatcaataatggagctatggcatgggctactggtggacatcaaggtaatcataaatgctag

SEQ ID NO: 234 - protein, 60 aa, Pediococcus pentosaceus
MTEIKVLNDKELKNVVGGKYYGNGVHCGKKTCYVDWGQATASIGKIIVNGWTQHGPWAHR

SEQ ID NO: 235 - DNA, 183 bp, Pediococcus pentosaceus
  source 1..183: mol_type = genomic DNA
atgactgaaattaaagtactaaacgataaggaactaaaaaatgtcgtaggaggaaagtattacggtaacggagtgcattgtggtaaaaagacttgctatgtggactggggacaagctacagctagcattggaaaaattatagtgaacggatggacacaacacgggccttgggcacatagataa

SEQ ID NO: 236 - protein, 60 aa, Staphylococcus epidermidis
MKNNKNLFDLEIKKETSQNTDELEPQTAGPAIRASVKQCQKTLKATRLFTVSCKGKNGCK

SEQ ID NO: 237 - DNA, 183 bp, Staphylococcus epidermidis
  source 1..183: mol_type = genomic DNA
atgaaaaataacaaaaatttatttgatttagaaattaaaaaagaaacaagtcaaaacactgatgaacttgaacctcaaactgctggaccagcgattagagcttctgtgaaacaatgtcagaaaactttgaaagctacgcgtttatttacagtgtcttgcaaaggaaaaaacggatgtaaatag

SEQ ID NO: 238 - protein, 62 aa, Carnobacterium maltaromaticum
MKTVKELSVKEMQLTTGGKYYGNGVSCNKNGCTVDWSKAIGIIGNNAAANLTTGGAAGWNKG

SEQ ID NO: 239 - DNA, 189 bp, Carnobacterium maltaromaticum
  source 1..189: mol_type = genomic DNA
atgaaaactgttaaagaacttagcgttaaagaaatgcaactaactacaggaggtaagtattacggaaatggcgtttcctgtaataaaaatggttgtactgtagattggagcaaagctattgggattataggaaacaatgcagcagcaaatttgactacaggtggagccgctggttggaacaaaggataa

SEQ ID NO: 240 - protein, 69 aa, Lactobacillus plantarum
  VARIANT 40: note = Xaa = any amino acid
MYKELTVDELALIDGGKKKKKKVACTWGNAATAAASGAVXGILGGPTGALAGAIWGVSQCASNNLHGMH

SEQ ID NO: 241 - DNA, 210 bp, Lactobacillus plantarum
  misc_feature 119: note = n = a, c, t or g
  source 1..210: mol_type = genomic DNA
atgtataaagaattaacagttgatgaattagcattgattgatggaggaaaaaagaagaagaaaaaagtagcttgtacttggggaaatgcagcaacagccgctgcttctggtgcagttangggtattcttggtgggcctactggtgcactggctggagctatctggggcgtttcacaatgcgcgtctaacaacttacacggcatgcactaa

SEQ ID NO: 242 - protein, 56 aa, Lactobacillus plantarum
MMKKIEKLTEKEMANIIGGKYYGNGVTCGKHSCSVNWGQAFSCSVSHLANFGHGKC

SEQ ID NO: 243 - DNA, 171 bp, Lactobacillus plantarum
  source 1..171: mol_type = genomic DNA
atgatgaaaaaaattgaaaaattaactgaaaaagaaatggccaatatcattggtggtaaatactatggtaatggggttacttgtggtaaacattcctgctctgttaactggggccaagcattttcttgtagtgtgtcacatttagctaacttcggtcatggaaagtgctaa

SEQ ID NO: 244 - protein, 64 aa, Lactobacillus plantarum
MSKLVKTLTVDEISKIQTNGGKPAWCWYTLAMCGAGYDSGTCDYMYSHCFGVKHSSGGGGSYHC

SEQ ID NO: 245 - DNA, 195 bp, Lactobacillus plantarum
  source 1..195: mol_type = genomic DNA
atgagtaaactagttaaaacattaactgtcgatgaaatctctaagattcaaaccaatggtggaaaacctgcatggtgttggtacacattggcaatgtgcggtgctggttatgattcaggcacttgtgattatatgtattcacactgctttggtgtaaaacactctagcggtggtggcggtagctaccattgttag

SEQ ID NO: 246 - protein, 56 aa, Lactobacillus plantarum
MLQFEKLQYSRLPQKKLAKISGGFNRGGYNFGKSVRHVVDAIGSVAGIRGILKSIR

SEQ ID NO: 247 - DNA, 171 bp, Lactobacillus plantarum
  source 1..171: mol_type = genomic DNA
atgctacagtttgagaaattacaatattccaggttgccgcaaaaaaagcttgccaaaatatctggtggttttaatcggggcggttataactttggtaaaagtgttcgacatgttgttgatgcaattggttcagttgcaggcattcgtggtattttgaaaagtattcgttaa

SEQ ID NO: 248 - protein, 52 aa, Lactobacillus plantarum
MKKFLVLRDRELNAISGGVFHAYSARGVRNNYKSAVGPADWVISAVRGFIHG

SEQ ID NO: 249 - DNA, 159 bp, Lactobacillus plantarum
  source 1..159: mol_type = genomic DNA
atgaaaaaatttctagttttgcgtgaccgtgaattaaatgctatttcaggtggcgttttccatgcctatagcgcgcgtggcgttcggaataattataaaagtgctgttgggcctgccgattgggtcattagcgctgtccgaggattcatccacggatag

SEQ ID NO: 250 - protein, 55 aa, Lactobacillus plantarum
MTVNKMIKDLDVVDAFAPISNNKLNGVVGGGAWKNFWSSLRKGFYDGEAGRAIRR

SEQ ID NO: 251 - DNA, 168 bp, Lactobacillus plantarum
  source 1..168: mol_type = genomic DNA
atgactgtgaacaaaatgattaaggatttggatgtagtagatgcatttgcacctatttctaataataagttgaacggggttgttgggggaggcgcttggaaaaatttctggtctagtttaagaaaaggattttatgatggcgaagctggcagagcaatccgtcgttaa

SEQ ID NO: 252 - protein, 57 aa, Lactobacillus plantarum
MKIKLTVLNEFEELTADAEKNISGGRRSRKNGIGYAIGYAFGAVERAVLGGSRDYNK

SEQ ID NO: 253 - DNA, 174 bp, Lactobacillus plantarum
  source 1..174: mol_type = genomic DNA
atgaaaattaaattaactgttttaaatgaatttgaagaattaactgctgacgctgaaaagaatatttctggtggccgtcggagtcgtaaaaatggaattggatacgctattggttatgcgtttggcgcggttgaacgggccgtgcttggtggttcaagggattataataagtga

SEQ ID NO: 254 - protein, 47 aa, Lactobacillus plantarum
MDKFEKISTSNLEKISGGDLTTKLWSSWGYYLGKKARWNLKHPYVQF

SEQ ID NO: 255 - DNA, 141 bp, Lactobacillus plantarum
  source 1..141: mol_type = genomic DNA
atggataaatttgaaaaaattagtacatctaacctagaaaagatctctggcggtgatttaacaaccaagttatggagctcttggggatattatcttggcaagaaagcacgttggaatttaaagcacccatatgttcaattt

SEQ ID NO: 256 - protein, 55 aa, Lactobacillus plantarum
MNNLNKFSTLGKSSLSQIEGGSVPTSVYTLGIKILWSAYKHRKTIEKSFNKGFYH

SEQ ID NO: 257 - DNA, 168 bp, Lactobacillus plantarum
  source 1..168: mol_type = genomic DNA
atgaataacttgaataaattttctactctaggcaagagtagcttgtctcaaattgagggcggatcagtcccaacttcagtatatacgcttggaattaaaattctatggtctgcgtataagcatcgcaaaacgattgaaaaaagttttaataaaggcttttatcattaa

SEQ ID NO: 258 - protein, 55 aa, Lactobacillus plantarum
MNNALSFEQQFTDFSTLSDSELESVEGGRNKLAYNMGHYAGKATIFGLAAWALLA

SEQ ID NO: 259 - DNA, 168 bp, Lactobacillus plantarum
  source 1..168: mol_type = genomic DNA
atgaataacgcattaagttttgaacaacaatttacagacttcagcaccttatcggactctgaattagaatccgttgagggtggccgaaataagcttgcatataatatggggcattacgctggtaaggcaaccatttttggacttgcagcatgggcactccttgcatga

SEQ ID NO: 260 - protein, 47 aa, Lactobacillus plantarum
MDKIIKFQGISDDQLNAVIGGKKKKQSWYAAAGDAIVSFGEGFLNAW

SEQ ID NO: 261 - DNA, 144 bp, Lactobacillus plantarum
  source 1..144: mol_type = genomic DNA
atggataagattattaagtttcaagggatttctgatgatcaattaaatgctgttatcggtgggaaaaagaaaaaacaatcttggtacgcagcagctggtgatgcaatcgttagttttggtgaaggatttttaaatgcttggtaa

SEQ ID NO: 262 - protein, 59 aa, Lactobacillus plantarum
MKISKIEAQARKDFFKKIDTNSNLLNVNGAKCKWWNISCDLGNNGHVCTLSHECQVSCN

SEQ ID NO: 263 - DNA, 180 bp, Lactobacillus plantarum
  source 1..180: mol_type = genomic DNA
atgaaaatttctaagattgaagctcaggctcgtaaagatttttttaaaaaaatcgatactaactcgaacttattaaatgtaaatggtgccaaatgcaagtggtggaatatttcgtgtgatttaggaaataatggccatgtttgtaccttgtcacatgaatgccaagtatcttgtaactaa

SEQ ID NO: 264 - protein, 67 aa, Lactobacillus plantarum
MTKTSRRKNAIANYLEPVDEKSINESFGAGDPEARSGIPCTIGAAVAASIAVCPTTKCSKRCGKRKK

SEQ ID NO: 265 - DNA, 204 bp, Lactobacillus plantarum
  source 1..204: mol_type = genomic DNA
atgactaaaactagtcgtcgtaagaatgctattgctaattatttagaaccagtcgacgaaaaaagtattaatgaatcttttggggctggggatccggaagcaagatccggaattccatgtacaatcggcgcagctgtcgcagcatcaattgcagtttgtccaactactaagtgtagtaaacgttgtggcaagcgtaagaaataa

SEQ ID NO: 266 - protein, 48 aa, Lactobacillus plantarum
MKIQIKGMKQLSNKEMQKIVGGKSSAYSLQMGATAIKQVKKLFKKWGW

SEQ ID NO: 267 - DNA, 147 bp, Lactobacillus plantarum
  source 1..147: mol_type = genomic DNA
atgaaaattcaaattaaaggtatgaagcaacttagtaataaggaaatgcaaaaaatagtaggtggaaagagtagtgcgtattctttgcagatgggggcaactgcaattaaacaggtaaagaaactgtttaaaaaatggggatggtaa

SEQ ID NO: 268 - protein, 207 aa, Propionibacterium jensenii
MNKTHKMATLVIAAILAAGMTAPTAYADSPGNTRITASEQSVLTQILGHKPTQTEYNRYVETYGSVPTEADINAYIEASESEGSSSQTAAHDDSTSPGTSTEIYTQAAPARFSMFFLSGTWITRSGVVSLSLKPRKGGIGNEGDERTWKTVYDKFHNAGQWTRYKNNGVDASMKKQYMCHFKYGMVKTPWNLEPHKKAADVSPVKCN

SEQ ID NO: 269 - DNA, 624 bp, Propionibacterium jensenii
  source 1..624: mol_type = genomic DNA
atgaacaaaacacacaaaatggcgacgctggtaattgccgcgatcttggccgccggaatgaccgcaccaactgcctatgcagattctcctggaaacaccagaattacagccagcgagcaaagcgtccttacccagatactcggccacaaacctacacaaactgaatataaccgatacgttgagacttacggaagcgtaccgaccgaagcagacatcaacgcatatatagaagcgtctgaatctgagggatcatcaagtcaaacggctgctcacgatgactcgacatcacccggcacgagtaccgaaatctacacgcaggcagcccctgccaggttctcaatgtttttcctgtccggaacttggatcactaggagtggtgtagtatcgctctccttgaagccaaggaagggtggtattggcaacgagggggacgagcgtacctggaagactgtatacgacaaattccataacgctgggcaatggacacgatacaagaacaacggcgtagacgccagcatgaaaaagcagtacatgtgccacttcaagtacgggatggtgaagacgccatggaatctggagccccacaagaaggctgcagacgtcagtccagtcaagtgcaactag

SEQ ID NO: 270 - protein, 96 aa, Propionibacterium thoenii
MKKTLLRSGTIALATAAAFGASLAAAPSAMAVPGGCTYTRSNRDVIGTCKTGSGQFRIRLDCNNAPDKTSVWAKPKVMVSVHCLVGQPRSISFETK

SEQ ID NO: 271 - DNA, 291 bp, Propionibacterium thoenii
  source 1..291: mol_type = genomic DNA
atgaagaagaccctcctgcgaagtggaacgatcgcactggcgaccgcggctgcatttggcgcatcattggcagccgccccatctgccatggccgttcctggtggttgcacgtacacaagaagcaatcgcgacgtcatcggtacctgcaagactggaagcggccagttccgaatccgacttgactgcaacaacgctccagacaaaacttcagtctgggccaagcccaaggtaatggtgtcggttcactgtcttgttggtcaaccgaggtccatctcgttcgagaccaagtga

SEQ ID NO: 272 - protein, 255 aa, Propionibacterium freudenreichii
  source 1..255: note = subsp freudenreii
MNTKAVNLKSENTTKLVSYLTENQLDEFIRRIRIDGALVEEVSQNAKQALDNTGLNGWINTDCDEGLLSDFISKIASARWIPLAESIRPAVTDRDKYRVSCWFYQGMNIAIYANIGGVANIIGYTEAAVATLLGAVVAVAPVVPGTPTPPKDKSSQYKEVPLAVRLSETYHEEGVRGLFDELNYSESRMISTLRRASTDGVLINSWNDGQDTILLKKYNFQDLQLTVRSRIVGNQTIIEECKITDGRKTLSDETV

SEQ ID NO: 273 - DNA, 768 bp, Propionibacterium freudenreichii
  source 1..768: mol_type = genomic DNA; note = subsp freudenreii
atgaataccaaagctgtaaatctgaagtcagaaaacacgactaagttggtgagctaccttacggaaaatcaattggatgagtttattagaaggattcgcattgatggcgctcttgtggaagaggtcagtcaaaatgctaagcaggccttagataatactgggctcaatggctggataaatactgattgcgatgaaggccttctctctgatttcatttcaaagatagcaagtgctagatggattccattagctgagtcaattcgacctgcggtgactgacagggataagtatcgagtaagttgctggttctaccaggggatgaatatagcaatttacgcaaatatcggtggcgtggccaatattatcggctatacggaggccgcagtcgcaacactccttggtgcagttgtggcggtagctcctgtggtccctggaactccaacccctccaaaggacaagagttcgcaatataaggaggttccccttgccgttcgtctttccgaaacataccacgaagagggagtacgaggtctattcgacgagctgaactactccgagagccgtatgatctctactctaaggcgagcatcaaccgatggagtcctaattaattcttggaacgatgggcaggatacaattctgcttaagaagtacaatttccaagacttgcaactgactgtcaggagccgcattgttgggaatcaaacaataattgaagaatgcaaaatcactgatggtagaaaaactctttcagacgagactgtgtag

SEQ ID NO: 274 - protein, 618 aa, Pseudomonas aeruginosa
MARPIADLIHFNSTTVTASGDVYYGPGGGTGIGPIARPIEHGLDSSTENGWQEFESYADVGVDPRRYVPLQVKEKRREIELQFRDAEKKLEASVQAELDKADAALGPAKNLAPLDVINRSLTIVGNALQQKNQKLLLNQKKITSLGAKNFLTRTAEEIGEQAVREGNINGPEAYMRFLDREMEGLTAAYNVKLFTEAISSLQIRMNTLTAAKASIEAAAANKAREQAAAEAKRKAEEQARQQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRVADGGGVYNMGNLVAVTPKRHIEIHKGGK

SEQ ID NO: 275 - DNA, 1857 bp, Pseudomonas aeruginosa
  source 1..1857: mol_type = genomic DNA
atggcacgacccattgctgaccttatccacttcaactctacaactgtcacggcaagcggagacgtttattacggccctgggggaggtaccggcattggccccattgccagacctatagagcacggcttggattcgtccactgaaaatggctggcaagagtttgaaagttatgctgatgtgggcgttgaccccagacgctatgttcctcttcaggttaaagaaaaacgcagggagatcgagcttcagttccgagatgccgagaaaaaacttgaggcgtcggtacaagccgagctggataaggctgatgccgctcttggtccggcaaagaatcttgcaccattggacgtcatcaaccgcagtctgaccatcgttggaaacgccctccagcaaaagaatcaaaaactactgctgaatcagaagaagattaccagcctgggtgcaaagaatttccttacccgtacggcggaagagatcggtgaacaagcggtgcgagaaggcaatattaacgggcctgaagcctatatgcgcttcctcgacagggaaatggaaggtctcacggcagcttataacgtaaaactcttcaccgaagcgatcagtagtctccagatccgcatgaatacgttgaccgccgccaaagcaagtattgaggcggccgcagcaaacaaggcgcgtgaacaagcagcggctgaggccaaacgcaaagccgaagagcaggcccgccagcaagcggcgataagagctgccaatacctatgccatgccggccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgagtagcagatggaggcggcgtttacaatatggggaaccttgttgcagtaacgccaaaacgtcatatagaaatccacaagggagggaagtga

SEQ ID NO: 276 - protein, 689 aa, Pseudomonas aeruginosa
MAVNDYEPGSMVITHVQGGGRDIIQYIPARSSYGTPPFVPPGPSPYVGTGMQEYRKLRSTLDKSHSELKKNLKNETLKEVDELKSEAGLPGKAVSANDIRDEKSIVDALMDAKAKSLKAIEDRPANLYTASDFPQKSESMYQSQLLASRKFYGEFLDRHMSELAKAYSADIYKAQIAILKQTSQELENKARSLEAEAQRAAAEVEADYKARKANVEKKVQSELDQAGNALPQLTNPTPEQWLERATQLVTQAIANKKKLQTANNALIAKAPNALEKQKATYNADLLVDEIASLQARLDKLNAETARRKEIARQAAIRAANTYAMPANGSVVATAAGRGLIQVAQGAASLAQAISDAIAVLGRVLASAPSVMAVGFASLTYSSRTAEQWQDQTPDSVRYALGMDAAKLGLPPSVNLNAVAKASGTVDLPMRLTNEARGNTTTLSVVSTDGVSVPKAVPVRMAAYNATTGLYEVTVPSTTAEAPPLILTWTPASPPGNQNPSSTTPVVPKPVPVYEGATLTPVKATPETYPGVITLPEDLIIGFPADSGIKPIYVMFRDPRDVPGAATGKGQPVSGNWLGAASQGEGAPIPSQIADKLRGKTFKNWRDFREQFWIAVANDPELSKQFNPGSLAVMRDGGAPYVRESEQAGGRIKIEIHHKVRIADGGGVYNMGNLVAVTPKRHIEIHKGGK

SEQ ID NO: 277 - DNA, 2070 bp, Pseudomonas aeruginosa
  source 1..2070: mol_type = genomic DNA
atggctgtcaatgattacgaacctggttcgatggttattacacatgtgcagggtggtgggcgtgacataatccagtatattcctgctcgatcaagctacggtactccaccatttgtcccaccaggaccaagtccgtatgtcggtactggaatgcaggagtacaggaagctaagaagtacgcttgataagtcccattcagaactcaagaaaaacctgaaaaatgaaaccctgaaggaggttgatgaactcaagagtgaagcggggttgccaggtaaagcggtcagtgccaatgacatccgcgatgaaaagagtatcgttgatgcactcatggatgccaaagcaaaatcgctaaaggccattgaggatcgcccggccaatctttatacggcttcagactttcctcagaagtcagagtcgatgtaccagagtcagttgctggccagccgaaaattctatggagagttcctggatcgccatatgagtgagctggccaaagcgtacagcgccgatatctataaggcgcaaatcgctatcttgaaacaaacgtctcaagagctggagaataaagcccggtcattggaagcagaagcccagcgagccgctgctgaggtggaggcggactacaaggccaggaaggcaaatgtcgagaaaaaagtgcagtccgagcttgaccaggctgggaatgctttgcctcaactgaccaatccaacgccagagcagtggcttgaacgcgctactcaactggttacgcaggcgatcgccaataagaagaaattgcagactgcaaacaatgccttgattgccaaggcacccaatgcactggagaaacaaaaggcaacctacaacgccgatctcctagtggatgaaatcgccagcctgcaagcacggctggacaagctgaacgccgaaacggcaaggcgcaaggaaatcgctcgtcaagcggcgatcagggctgccaatacttatgccatgccagccaatggcagcgttgtcgccaccgccgcaggccggggtctgatccaggtcgcacaaggcgccgcatcccttgctcaagcgatctccgatgcgattgccgtcctgggccgggtcctggcttcagcaccctcggtgatggccgtgggctttgccagtctgacctactcctcccggactgccgagcaatggcaggaccaaacgcccgatagcgttcgttacgccctgggcatggatgccgctaaattggggcttcccccaagcgtaaacctgaacgcggttgcaaaagccagcggtaccgtcgatctgccgatgcgcctgaccaacgaggcacgaggcaacacgacgaccctttcggtggtcagcaccgatggtgtgagcgttccgaaagccgttccggtccggatggcggcctacaatgccacgacaggcctgtacgaggttacggttccctctacgaccgcagaagcgccgccactgatcctgacctggacgccggcgagtcctccaggaaaccagaacccttcgagtaccactccggtcgtaccgaagccggtgccggtatatgagggagcgacccttacaccggtgaaggctaccccggaaacctatcctggggtgattacactaccggaagacctgatcatcggcttcccggccgactcggggatcaagccgatctatgtgatgttcagggatccgcgggatgtacctggtgctgcgactggcaagggacagcccgtcagcggtaattggctcggcgccgcctctcaaggtgagggggctccaattccaagccagattgcggataaactacgtggtaagacattcaaaaactggcgggactttcgggaacaattctggatagctgtggctaatgatcctgagttaagtaaacagtttaatcctggtagtttagctgtaatgagagatggaggggctccttatgtcagagagtcagaacaggctggcgggagaataaagatcgaaatccaccacaaggttcgaatagcagatggaggcggcgtttacaatatggggaaccttgttgcagtaacgccaaaacgtcatatagaaatccacaagggagggaagtga

SEQ ID NO: 278 - protein, 47 aa, Ruminococcus gnavus
MRNDVLTLTNPMEEKELEQILGGGNGVLKTISHECNMNTWQFLFTCC

SEQ ID NO: 279 - DNA, 144 bp, Ruminococcus gnavus
  source 1..144: mol_type = genomic DNA
atgagaaatgacgtattaacattaacaaacccaatggaagagaacgaactggagcagatcttaggtggtggcaatggtgtgttaaaaacgattagccacgaatgcaatatgaacacatggcagttcctgtttacttgttgctaa

SEQ ID NO: 280 - protein, 55 aa, Lactobacillus sakei
MKNAKSLTIQEMKSITGGKYYGNGVSCNSHGCSVNWGQAWTCGVNHLANGGHGVC

SEQ ID NO: 281 - DNA, 168 bp, Lactobacillus sakei
  source 1..168: mol_type = genomic DNA
atgaaaaacgcaaaaagcctaacaattcaagaaatgaaatctattacaggtggtaaatactatggtaatggcgttagctgtaactctcacggctgttcagtaaattgggggcaagcatggacttgtggagtaaaccatctagctaatggcggtcatggagtttgttaa

SEQ ID NO: 282 - protein, 59 aa, Lactobacillus sakei; identical to SEQ ID NO: 68

SEQ ID NO: 283 - DNA, 180 bp, Lactobacillus sakei; identical to SEQ ID NO: 69
  source 1..180: mol_type = genomic DNA

SEQ ID NO: 284 - protein, 61 aa, Lactobacillus sakei
MEKFIELSLKEVTAITGGKYYGNGVHCGKHSCTVDWGTAIGNIGNNAAANWATGGNAGWNK

SEQ ID NO: 285 - DNA, 186 bp, Lactobacillus sakei
  source 1..186: mol_type = genomic DNA
atggaaaagtttattgaattatctttaaaagaagtaacagcaattacaggtggaaaatattatggtaacggtgtacactgtggaaaacattcatgtaccgtagactggggaacagctattggaaatatcggaaataatgcagctgcaaactgggccacaggcggaaacgctggctggaataaataa

SEQ ID NO: 286 - protein, 56 aa, Streptococcus salivarius
MKSTNNQSIAEIAAVNSLQEVSMEELDQIIGAGNGVVLTLTHECNLATWTKKLKCC

SEQ ID NO: 287 - DNA, 171 bp, Streptococcus salivarius
  source 1..171: mol_type = genomic DNA
atgaaatcaacaaataatcaaagtatcgcagaaattgcagcagtaaactcactacaagaagtaagtatggaggaactagaccaaattattggtgccggaaacggagtggttcttactcttactcatgaatgtaacctagcaacttggacaaaaaaactaaaatgttgctaa

SEQ ID NO: 288 - protein, 51 aa, Streptococcus pyogenes
  source 1..51: note = serotype M28
MSFMKNSKDILTNAIEEVSEKELMEVAGGKKGSGWFATITDDCPNSVFVCC

SEQ ID NO: 289 - DNA, 156 bp, Streptococcus pyogenes
  source 1..156: mol_type = genomic DNA; serotype = M28
atgagttttatgaaaaattcaaaggatattttgactaatgctatcgaagaagtttctgaaaaagaacttatggaagtagctggtggtaaaaaaggttccggttggtttgcaactattactgatgactgtccgaactcagtattcgtttgttgttaa

SEQ ID NO: 290 - protein, 48 aa, Streptococcus salivarius
MKNSKDVLNNAIEEVSEKELMEVAGGKKGPGWIATITDDCPNSIFVCC

SEQ ID NO: 291 - DNA, 147 bp, Streptococcus salivarius
  source 1..147: mol_type = genomic DNA
atgaaaaactcaaaagatgttttgaacaatgctatcgaagaggtttctgaaaaagaacttatggaagtagctggtggtaaaaaaggtccaggttggattgcaactattactgatgactgtccaaactcaatattcgtttgttgttaa

SEQ ID NO: 292 - protein, 48 aa, Streptococcus salivarius
MKNSKDILNNAIEEVSEKELMEVAGGKRGSGWIATITDDCPNSVFVCC

SEQ ID NO: 293 - DNA, 147 bp, Streptococcus salivarius
  source 1..147: mol_type = genomic DNA
atgaaaaactcaaaagatattttgaacaatgctatcgaagaagtttctgaaaaagaacttatggaagtagctggtggtaaaagaggttcaggttggattgcaactattactgatgactgtccaaactcagtattcgtttgttgttaa

SEQ ID NO: 294 - protein, 62 aa, Staphylococcus aureus
MKSSFLEKDIEEQVTWFEEVSEQEFDDDIFGACSTNTFSLSDYWGNKGNWCTATHECMSWCK

SEQ ID NO: 295 - DNA, 189 bp, Staphylococcus aureus
  source 1..189: mol_type = genomic DNA
atgaaaagttcttttttagaaaaagatatagaagaacaagtgacatggttcgaggaagtttcagaacaagaatttgacgatgatatttttggagcttgtagtacaaacactttttctttgagtgactattggggtaataaaggaaattggtgtactgctactcacgaatgtatgtcttggtgtaaataa

SEQ ID NO: 296 - protein, 67 aa, Staphylococcus aureus
MKNELGKFLEENELELGKFSESDMLEITDDEVYAAGTPLALLGGAATGVIGYISNQTCPTTACTRAC

SEQ ID NO: 297 - DNA, 204 bp, Staphylococcus aureus
  source 1..204: mol_type = genomic DNA
atgaaaaatgaattaggtaagtttttagaagaaaacgaattagagttaggtaaattttcagaatcagacatgctagaaattactgatgatgaagtatatgcagctggaacacctttagccttattgggtggagctgccaccggggtgataggttatatttctaaccaaacatgtccaacaactgcttgtacacgcgcttgctag

SEQ ID NO: 298 - protein, 46 aa, Streptococcus pyogenes
MNNTIKDFDLDLKTNKKDTATPYVGSRYLCTPGSCWKLVCFTTTVK

SEQ ID NO: 299 - DNA, 141 bp, Streptococcus pyogenes
  source 1..141: mol_type = genomic DNA
atgaataacacaattaaagactttgatctcgatttgaaaacaaataaaaaagacactgctacaccttatgttggtagccgttacctatgtacccctggttcttgttggaaattagtttgctttacaacaactgttaaataa

SEQ ID NO: 300 - protein, 51 aa, Streptococcus pyogenes
MEKNNEVINSIQEVSLEELDQIIGAGKNGVFKTISHECHLNTWAFLATCCS

SEQ ID NO: 301 - DNA, 156 bp, Streptococcus pyogenes
  source 1..156: mol_type = genomic DNA
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgttcataa

SEQ ID NO: 302 - protein, 51 aa, Streptococcus pyogenes
  source 1..51: note = serotype M49
MTKEHEIINSIQEVSLEELDQIIGAGKNGVFKTISHECHLNTWAFLATCCS

SEQ ID NO: 303 - DNA, 156 bp, Streptococcus pyogenes
  source 1..156: mol_type = genomic DNA; serotype = M49
atggaaaaaaataatgaagtaatcaactctattcaagaagttagtcttgaagaactcgatcaaattatcggtgctggaaaaaatggtgtgtttaaaacaatttctcatgagtgtcatttgaatacatgggcattccttgctacttgttgctcataa

SEQ ID NO: 304 - protein, 56 aa, Bacillus subtilis
MEKLFKEVKLEELENQKGSGLGKAQCAALWLQCASGGTIGCGGGAVACQNYRQFCR

SEQ ID NO: 305 - DNA, 171 bp, Bacillus subtilis
  source 1..171: mol_type = genomic DNA
atggaaaagctatttaaagaagttaaactagaggaactcgaaaaccaaaaaggtagtggattaggaaaagctcagtgtgctgcgttgtggctacaatgtgctagtggcggtacaattggttgtggtggcggagctgttgcttgtcaaaactatcgtcaattctgcagataa

SEQ ID NO: 306 - protein, 56 aa, Bacillus subtilis
MSKFDDFDLDVVKVSKQDSKITPQWKSESLCTPGCVTGALQTCFLQTLTCNCKISK

SEQ ID NO: 307 - DNA, 171 bp, Bacillus subtilis
  source 1..171: mol_type = genomic DNA
atgtcaaagttcgatgatttcgatttggatgttgtgaaagtctctaaacaagactcaaaaatcactccgcaatggaaaagtgaatcactttgtacaccaggatgtgtaactggtgcattgcaaacttgcttccttcaaacactaacttgtaactgcaaaatctctaaataa

SEQ ID NO: 308 - protein, 50 aa, Bacillus subtilis
MKLPVQQVYSVYGGKDLPKGHSHSTMPFLSKLQFLTKIYLLDIHTQPFFI

SEQ ID NO: 309 - DNA, 153 bp, Bacillus subtilis
  source 1..153: mol_type = genomic DNA
ttgaaattgccggtgcaacaggtctattcggtctatgggggtaaggatctcccaaaagggcatagtcattctactatgccctttttaagtaaattacaatttttaactaaaatctacctcttggatatacatacacaaccgtttttcatttga

SEQ ID NO: 310 - protein, 43 aa, Bacillus subtilis
MKKAVIVENKGCATCSIGAACLVDGPIPDFEIAGATGLFGLWG

SEQ ID NO: 311 - DNA, 132 bp, Bacillus subtilis
  source 1..132: mol_type = genomic DNA
atgaaaaaagctgtcattgtagaaaacaaaggttgtgcaacatgctcgatcggagccgcttgtctagtggacggtcctatccctgattttgaaattgccggtgcaacaggtctattcggtctatgggggtaa

SEQ ID NO: 312 - protein, 58 aa, Streptococcus thermophilus; identical to SEQ ID NO: 24

SEQ ID NO: 313 - DNA, 177 bp, Streptococcus thermophilus
  source 1..177: mol_type = genomic DNA
atgatgaatgctactgaaaaccaaatttttgttgagactgtgagtgaccaagaattagaaatgttaattggtggtgcagatcgtggatggattaagactttaacaaaagattgtccaaatgtaatttcttcaatttgtgcaggtacaattattacagcctgtaaaaattgtgcttaa

SEQ ID NO: 314 - protein, 64 aa, Streptococcus thermophilus
MKQYNGFEVLHELDLANVTGGQINWGSVVGHCIGGAIIGGAFSGGAAAGVGCLVGSGKAIINGL

SEQ ID NO: 315 - DNA, 195 bp, Streptococcus thermophilus
  source 1..195: mol_type = genomic DNA
atgaagcagtataatggttttgaggttctacatgaacttgacttagcaaatgtaactggcggtcaaattaattggggatcagttgtaggacactgtataggtggagctattatcggaggtgcattttcaggaggtgcagcggctggagtaggatgccttgttgggagcggaaaggcaatcataaatggattataa

SEQ ID NO: 316 - protein, 85 aa, Streptococcus thermophilus
MNTITICKFDVLDAELLSTVEGGYSGKDCLKDMGGYALAGAGSGALWGAPAGGVGALPGAFVGAHVGAIAGGFACMGGMIGNKFN

SEQ ID NO: 317 - DNA, 258 bp, Streptococcus thermophilus
  source 1..258: mol_type = genomic DNA
atgaatacaataactatttgtaaatttgatgttttagatgctgaacttctttcgacagttgagggtggatactctggtaaggattgtttaaaagacatgggaggatatgcattggcaggagctggaagtggagctctgtggggagctccagcaggaggtgttggagcacttccaggtgcatttgtcggagctcatgttggggcaattgcaggaggctttgcatgtatgggtggaatgattggtaataagtttaactaa

SEQ ID NO: 318 - protein, 52 aa, Bacillus cereus
MSEIKKALNTLEIEDFDAIEMVDVDAMPENEALEIMGASCTTCVCTCSCCTT

SEQ ID NO: 319 - DNA, 159 bp, Bacillus cereus
  source 1..159: mol_type = genomic DNA
atgagtgaaattaaaaaagcattaaatacgcttgaaattgaagattttgatgcaattgaaatggttgatgttgatgctatgccagaaaacgaagcgcttgaaattatgggagcgtcatgtacgacatgcgtatgtacatgcagttgttgtacaacttga

SEQ ID NO: 320 - protein, 47 aa, Bacillus cereus
MEVMNNALITKVDEEIGGNAACVIGCIGSCVISEGIGSLVGTAFTLG

SEQ ID NO: 321 - DNA, 144 bp, Bacillus cereus
  source 1..144: mol_type = genomic DNA
atggaagttatgaacaatgctttaattacaaaagtagatgaggagattggaggaaacgctgcttgtgtaattggttgtattggcagttgcgtaattagtgaaggaattggttcacttgtaggaacagcatttactttaggttaa

SEQ ID NO: 322 - protein, 49 aa, Bacillus cereus
MEVLNKQNVNIIPESEEVGGWVACVGACGTVCLASGGVGTEFAAASYFL

SEQ ID NO: 323 - DNA, 150 bp, Bacillus cereus
  source 1..150: mol_type = genomic DNA
atggaagttttaaacaaacaaaatgtaaatattattccagaatctgaagaagtaggtggatgggtagcatgtgttggagcatgtggtacagtatgtcttgctagtggtggtgttggaacagagtttgcagctgcatcttatttcctataa

SEQ ID NO: 324 - protein, 40 aa, Bacillus thuringiensis
METPVVQPRDWTCWSCLVCAACSVELLNLVTAATGASTAS

SEQ ID NO: 325 - DNA, 123 bp, Bacillus thuringiensis
  source 1..123: mol_type = genomic DNA
atggaaacaccagtagtacaaccaagggattggacttgttggagttgcttagtatgtgcagcatgttctgtggaattattaaatttagttactgcggcaacaggggctagtactgcaagctaa

SEQ ID NO: 326 - protein, 42 aa, Rhizobium leguminosarum
  source 1..42: note = bv. trifolii
MDNKVAKNVEVKKGSIKATFKAAVLKSKTKVDIGGSRQGCVA

SEQ ID NO: 327 - DNA, 129 bp, Rhizobium leguminosarum
  source 1..129: mol_type = genomic DNA; note = bv. trifolii
atggataacaaggttgcgaagaatgtcgaagtgaagaagggctccatcaaggcgaccttcaaggctgctgttctgaagtcgaagacgaaggtcgacatcggaggtagccgtcagggctgcgtcgcttaa

SEQ ID NO: 328 - protein, 70 aa, Streptococcus uberis
MNTIEKFENIKLFSLKKIIGGKTVNYGNGLYCNQKKCWVNWSETATTIVNNSIMNGLTGGNAGWHSGGRA

SEQ ID NO: 329 - DNA, 213 bp, Streptococcus uberis
  source 1..213: mol_type = genomic DNA
atgaatacaattgaaaaatttgaaaatattaaacttttttcactaaagaaaattatcggtggcaaaactgtaaattatggtaatggcctttattgtaaccaaaaaaaatgctgggtaaactggtcagaaactgctacaacaatagtaaataattccatcatgaacgggctcacaggtggtaatgcgggttggcactcaggcgggagagcataa

SEQ ID NO: 330 - protein, 76 aa, Streptococcus uberis
MDILLELAGYTGIASGTAKKVVDAIDKGAAAFVIISIISTVISAGALGAVSASADFIILTVKNYISRNLKAQAVIW

SEQ ID NO: 331 - DNA, 231 bp, Streptococcus uberis
  source 1..231: mol_type = genomic DNA
atggacattttattagaactcgcaggatatactgggatagcctcaggtactgcaaaaaaagttgttgatgccattgataaaggagctgcagcctttgttattatttcaattatctcaacagtaattagtgcgggagcattgggagcagtttcagcctcagctgattttattattttaactgtaaaaaattacattagtagaaatttaaaagcacaagctgtcatttggtaa

SEQ ID NO: 332 - protein, 64 aa, Clostridium perfringens
MDSELFKLMATQGAFAILFSYLLFYVLKENSKREDKYQNIIEELTELLPKIKEDVEDIKEKLNK

SEQ ID NO: 333 - DNA, 195 bp, Clostridium perfringens
  source 1..195: mol_type = genomic DNA
atggatagtgaattatttaagttaatggcaacacaaggagcctttgcaatattattttcgtatttattgttttatgttttaaaagagaatagtaaaagagaagataagtatcaaaatataatagaggagcttacagaattattgccaaaaataaaagaagatgtagaagatataaaagaaaaacttaataaatag

SEQ ID NO: 334 - protein, 47 aa, Micrococcus varians
MTNAFQALDEVTDAELDAILGGGSGVIPTISHECHMNSFQFVFTCCS

SEQ ID NO: 335 - DNA, 144 bp, Micrococcus varians
  source 1..144: mol_type = genomic DNA
atgacgaacgcatttcaggcactggacgaagtcacggacgccgagctcgacgccatccttggcgggggcagtggtgttattcccacgatcagccacgagtgccacatgaactccttccagttcgtgttcacctgctgctcctga

SEQ ID NO: 336 - protein, 285 aa, Streptococcus equi
  source 1..285: note = subsp. zooepidemicus
MKRIFFAFLSLCLFIFGTQTVSAATYTRPLDTGNITTGFNGYPGHVGVDYAVPVGTPVRAVANGTVKFAGNGANHPWMLWMAGNCVLIQHADGMHTGYAHLSKISVSTDSTVKQGQIIGYTGATGQVTGPHLHFEMLPANPNWQNGFSGRIDPTGYIANAPVFNGTTPTEPTTPTTNLKIYKVDDLQKINGIWQVRNNILVPTDFTWVDNGIAADDVIEVTSNGTRTSDQVLQKGGYFVINPNNVKSVGTPMKGSGGLSWAQVNFTTGGNVWLNTTSKDNLLYGK

SEQ ID NO: 337 - DNA, 858 bp, Streptococcus equi
  source 1..858: mol_type = genomic DNA; note = subsp. zooepidemicus
atgaaacgtatattttttgctttcttaagtttatgcttatttatattcggaacacaaacggtatctgcagctacttatactcggccattagatacgggaaatatcactacagggtttaacggataccctggtcatgttggagtcgattatgcagtacccgttggaactccggttagagcagttgcaaatggtacagtcaaatttgcaggtaatggggctaatcacccatggatgctttggatggctggaaactgtgttctaattcaacatgctgacgggatgcatactggatatgcacacttatcaaaaatttcagttagcacagatagtacagttaaacaaggacaaatcataggttatactggtgccaccggccaagttaccggtccacatttgcattttgaaatgttgccagcaaatcctaactggcaaaatggtttttctggaagaatagatccaaccggatacatcgctaatgcccctgtatttaatggaacaacacctacagaacctactactcctacaacaaatttaaaaatctataaagttgatgatttacaaaaaattaatggtatttggcaagtaagaaataacatacttgtaccaactgatttcacatgggttgataatggaattgcagcagatgatgtaattgaagtaactagcaatggaacaagaacctctgaccaagttcttcaaaaaggtggttattttgtcatcaatcctaataatgttaaaagtgttggaactccgatgaaaggtagtggtggtctatcttgggctcaagtaaactttacaacaggtggaaatgtctggttaaatactactagcaaagacaacttactttacggaaaataa

SEQ ID NO: 338 - protein, 45 aa, Myxococcus fulvus
ANCSCSTASDYCPILTFCTTGTACSYTPTGCGTGWVYCACNGNFY

SEQ ID NO: 339 - DNA, 135 bp, synthetic construct
  misc_feature 1..135: note = Polynucleotide encoding the polypeptide of SEQ ID NO: 338
  source 1..135: mol_type = other DNA
gcgaactgcagctgcagcaccgcgagcgattattgcccgattctgaccttttgcaccaccggcaccgcgtgcagctataccccgaccggctgcggcaccggctgggtgtattgcgcgtgcaacggcaacttttat

SEQ ID NO: 340 - protein, 19 aa, Streptomyces griseoluteus
CANSCSYGPLTWSCDGNTK

SEQ ID NO: 341 - DNA, 57 bp, synthetic construct
  misc_feature 1..57: note = Polynucleotide encoding the polypeptide of SEQ ID NO: 340
  source 1..57: mol_type = other DNA
tgcgcgaacagctgcagctatggcccgctgacctggagctgcgatggcaacaccaaa

SEQ ID NO: 342 - protein, 19 aa, Streptoverticillium griseoverticillatum
CKQSCSFGPFTFVCDGNTK

SEQ ID NO: 343 - DNA, 57 bp, synthetic construct
  misc_feature 1..57: note = Polynucleotide encoding the polypeptide of SEQ ID NO: 342
  source 1..57: mol_type = other DNA
tgcaaacagagctgcagctttggcccgtttacctttgtgtgcgatggcaacaccaaa

SEQ ID NO: 344 - protein, 7 aa, Carnobacterium sp.
GSEIQPR

SEQ ID NO: 345 - DNA, 21 bp, synthetic construct
  misc_feature 1..21: note = Polynucleotide encoding the polypeptide of SEQ ID NO: 344
  source 1..21: mol_type = other DNA
ggcagcgaaattcagccgcgc

SEQ ID NO: 346 - protein, 39 aa, Lactococcus lactis
  source 1..39: note = subsp. lactis
GTWDDIGQGIGRVAYWVGKAMGNMSDVNQASRINRKKKH

SEQ ID NO: 347 - DNA, 117 bp, synthetic construct
  misc_feature 1..117: note = Polynucleotide encoding the polypeptide of SEQ ID NO: 346
  source 1..117: mol_type = other DNA
ggcacctgggatgatattggccagggcattggccgcgtggcgtattgggtgggcaaagcgatgggcaacatgagcgatgtgaaccaggcgagccgcattaaccgcaaaaaaaaacat

SEQ ID NO: 348 - protein, 35 aa, Lactococcus lactis
  source 1..35: note = subsp. lactis
KKWGWLAWVDPAYEFIKGFGKGAIKEGNKDKWKNI

SEQ ID NO: 349 - DNA, 105 bp, synthetic construct
  misc_feature 1..105: note = Polynucleotide encoding the polypeptide of SEQ ID NO: 348
  source 1..105: mol_type = other DNA
aaaaaatggggctggctggcgtgggtggatccggcgtatgaatttattaaaggctttggcaaaggcgcgattaaagaaggcaacaaagataaatggaaaaacatt

SEQ ID NO: 350 - protein, 19 aa, Streptomyces sp.
CVQSCSFGPLTWSCDGNTK

SEQ ID NO: 351 - DNA, 57 bp, synthetic construct
  misc_feature 1..57: note = Polynucleotide encoding the polypeptide of SEQ ID NO: 350
  source 1..57: mol_type = other DNA
tgcgtgcagagctgcagctttggcccgctgacctggagctgcgatggcaacaccaaa

SEQ ID NO: 352 - protein, 19 aa, Actinoplanes liguriae
SSGWVCTLTIECGTVICAC

SEQ ID NO: 353 - DNA, 57 bp, synthetic construct
  misc_feature 1..57: note = Polynucleotide encoding the polypeptide of SEQ ID NO: 352
  source 1..57: mol_type = other DNA
agcagcggctgggtgtgcaccctgaccattgaatgcggcaccgtgatttgcgcgtgc

SEQ ID NO: 354 - protein, 38 aa, Lactobacillus curvatus
  VARIANT 34: note = Xaa = any amino acid
  VARIANT 37: note = Xaa = any amino acid
YTAKQCLQAIGSCGIAGTGAGAAGGPAGAFVGAXVVXI

SEQ ID NO: 355 - DNA, 114 bp, synthetic construct
  misc_feature 1..114: note = Polynucleotide encoding the polypeptide of SEQ ID NO: 354
  misc_difference 100..102: note = n is A, T, C or G
  misc_difference 100..102: note = nnn is a codon other than a stop codon
  misc_difference 109..111: note = nnn is a codon other than a stop codon
  source 1..114: mol_type = other DNA
  misc_difference 109..111: note = n is A, T, C, or G
tataccgcgaaacagtgcctgcaggcgattggcagctgcggcattgcgggcaccggcgcgggcgcggcgggcggcccggcgggcgcgtttgtgggcgcgnnngtggtgnnnatt

SEQ ID NO: 356 - protein, 42 aa, Lactobacillus sakei
  VARIANT 32: note = Xaa = any amino acid
TKYYGNGVYCNSKKCWVDWGQAAGGIGQTVVXGWLGGAIPGK

SEQ ID NO: 357 - DNA, 126 bp, synthetic construct
  misc_feature 1..126: note = Polynucleotide encoding the polypeptide of SEQ ID NO: 356
  misc_feature 94..96: note = nnn = any amino acid-coding triplet
  source 1..126: mol_type = other DNA
accaaatattatggcaacggcgtgtattgcaacagcaaaaaatgctgggtggattggggccaggcggcgggcggcattggccagaccgtggtgnnnggctggctgggcggcgcgattccgggcaaa

SEQ ID NO: 358 - protein, 22 aa, Streptococcus mutans
FKSWSFCTPGCAKTGSFNSYCC

SEQ ID NO: 359 - DNA, 132 bp, synthetic construct
  misc_feature 1..132: note = Polynucleotide encoding the polypeptide of SEQ ID NO: 358
  source 1..132: mol_type = other DNA
tttaaaagctggagcttttgcaccccgggctgcgcgaaaaccggcagctttaacagctattgctgctttaaaagctggagcttttgcaccccgggctgcgcgaaaaccggcagctttaacagctattgctgc

SEQ ID NO: 360 - protein, 43 aa, Enterococcus mundtii
KYYGNGVSCNKKGCSVDWGKAIGIIGNNSAANLATGGAAGWSK

SEQ ID NO: 361 - DNA, 129 bp, synthetic construct
  misc_feature 1..129: note = Polynucleotide encoding the polypeptide of SEQ ID NO: 360
  source 1..129: mol_type = other DNA
aaatattatggcaacggcgtgagctgcaacaaaaaaggctgcagcgtggattggggcaaagcgattggcattattggcaacaacagcgcggcgaacctggcgaccggcggcgcggcgggctggagcaaa

SEQ ID NO: 362 - protein, 41 aa, Lactobacillus sakei
  VARIANT 9: note = Xaa = any amino acid
  VARIANT 14: note = Xaa is any amino acid
  VARIANT 33: note = Xaa is any amino acid
  VARIANT 37: note = Xaa is any amino acid
KYYGNGVHXGKHSXTVDWGTAIGNIGNNAAANXATGXNAGG

SEQ ID NO: 363 - DNA, 123 bp, synthetic construct
  misc_feature 1..123: note = Polynucleotide encoding the polypeptide of SEQ ID NO: 362
  misc_feature 25..27: note = nnn = any amino acid-coding triplet
  misc_feature 40..42: note = nnn = any amino acid-coding triplet
  misc_feature 97..99: note = nnn = any amino acid-coding triplet
  misc_feature 109..111: note = nnn = any amino acid-coding triplet
  source 1..123: mol_type = other DNA
aaatattatggcaacggcgtgcatnnnggcaaacatagcnnnaccgtggattggggcaccgcgattggcaacattggcaacaacgcggcggcgaacnnngcgaccggcnnnaacgcgggcggc

SEQ ID NO: 364 - protein, 31 aa, Lactobacillus paracasei
GMSGYIQGIPDFLKGYLHGISAANKHKKGRL

SEQ ID NO: 365 - DNA, 93 bp, synthetic construct
  misc_feature 1..93: note = Polynucleotide encoding the polypeptide of SEQ ID NO: 364
  source 1..93: mol_type = other DNA
ggcatgagcggctatattcagggcattccggattttctgaaaggctatctgcatggcattagcgcggcgaacaaacataaaaaaggccgcctg

SEQ ID NO: 366 - protein, 31 aa, Leuconostoc mesenteroides
KGKGFWSWASKATSWLTGPQQPGSPLLKKHR

SEQ ID NO: 367 - DNA, 93 bp, synthetic construct
  misc_feature 1..93: note = Polynucleotide encoding the polypeptide of SEQ ID NO: 366
  source 1..93: mol_type = other DNA
aaaggcaaaggcttttggagctgggcgagcaaagcgaccagctggctgaccggcccgcagcagccgggcagcccgctgctgaaaaaacatcgc

SEQ ID NO: 368 - protein, 43 aa, Leuconostoc mesenteroides
KNYGNGVHCTKKGCSVDWGYAWTNIANNSVMNGLTGGNAGWHN

SEQ ID NO: 369 - DNA, 129 bp, synthetic construct
  misc_feature 1..129: note = Polynucleotide encoding the polypeptide of SEQ ID NO: 368
  source 1..129: mol_type = other DNA
aaaaactatggcaacggcgtgcattgcaccaaaaaaggctgcagcgtggattggggctatgcgtggaccaacattgcgaacaacagcgtgatgaacggcctgaccggcggcaacgcgggctggcataac

SEQ ID NO: 370 - protein, 47 aa, Bacillus subtilis
AIKLVQSPNGNFAASFVLDGTKWIFKSKYYDSSKGYWVGIYEVWDRK

SEQ ID NO: 371 - DNA, 141 bp, synthetic construct
  misc_feature 1..141: note = Polynucleotide encoding the polypeptide of SEQ ID NO: 370
  source 1..141: mol_type = other DNA
gcgattaaactggtgcagagcccgaacggcaactttgcggcgagctttgtgctggatggcaccaaatggatttttaaaagcaaatattatgatagcagcaaaggctattgggtgggcatttatgaagtgtgggatcgcaaa

SEQ ID NO: 372 - protein, 12 aa, Bacillus licheniformis
  VARIANT 7: note = Xaa = any amino acid
ISLEICXIFHDN

SEQ ID NO: 373 - DNA, 36 bp, synthetic construct
  misc_feature 1..36: note = Polynucleotide encoding the polypeptide of SEQ ID NO: 372
  misc_feature 19..21: note = nnn = any amino acid-coding triplet
  source 1..36: mol_type = other DNA
attagcctggaaatttgcnnnatttttcatgataac

SEQ ID NO: 374 - protein, 37 aa, Lactococcus lactis
  source 1..37: note = subsp. lactis
TSYGNGVHCNKSKCWIDVSELETYKAGTVSNPKDILW

SEQ ID NO: 375 - DNA, 111 bp, synthetic construct
  misc_feature 1..111: note = Polynucleotide encoding the polypeptide of SEQ ID NO: 374
  source 1..111: mol_type = other DNA
accagctatggcaacggcgtgcattgcaacaaaagcaaatgctggattgatgtgagcgaactggaaacctataaagcgggcaccgtgagcaacccgaaagatattctgtgg

SEQ ID NO: 376 - protein, 9 aa, Serratia plymuthica
DYHHGVRVL

SEQ ID NO: 377 - DNA, 27 bp, synthetic construct
  misc_feature 1..27: note = Polynucleotide encoding the polypeptide of SEQ ID NO: 376
  source 1..27: mol_type = other DNA
gattatcatcatggcgtgcgcgtgctg

SEQ ID NO: 378 - protein, 15 aa, Halobacterium sp.
DIDITGCSACKYAAG

SEQ ID NO: 379 - DNA, 45 bp, synthetic construct
  misc_feature 1..45: note = Polynucleotide encoding the polypeptide of SEQ ID NO: 378
  source 1..45: mol_type = other DNA
gatattgatattaccggctgcagcgcgtgcaaatatgcggcgggc

SEQ ID NO: 380 - protein, 12 aa, Bacillus subtilis
  VARIANT 1..2: note = Xaa = any amino acid
  VARIANT 6: note = Xaa is any amino acid
XXKEIXHIFHDN

SEQ ID NO: 381 - DNA, 36 bp, synthetic construct
  misc_feature 1..36: note = Polynucleotide encoding the polypeptide of SEQ ID NO: 380
  misc_feature 1..3: note = nnn = any amino acid-coding triplet
  misc_feature 4..6: note = nnn = any amino acid-coding triplet
  misc_feature 16..18: note = nnn = any amino acid-coding triplet
  source 1..36: mol_type = other DNA
nnnnnnaaagaaattnnncatatttttcatgataac

SEQ ID NO: 382 - protein, 12 aa, Lactobacillus curvatus
TPVVNPPFLQQT

SEQ ID NO: 383 - DNA, 36 bp, synthetic construct
  misc_feature 1..36: note = Polynucleotide encoding the polypeptide of SEQ ID NO: 382
  source 1..36: mol_type = other DNA
accccggtggtgaacccgccgtttctgcagcagacc

SEQ ID NO: 384 - protein, 10 aa, Lactobacillus curvatus
  VARIANT 10: note = Xaa = any amino acid
VAPFPEQFLX

SEQ ID NO: 385 - DNA, 30 bp, synthetic construct
  misc_feature 1..30: note = Polynucleotide encoding the polypeptide of SEQ ID NO: 384
  misc_feature 28..30: note = nnn = any amino acid-coding triplet
  source 1..30: mol_type = other DNA
gtggcgccgtttccggaacagtttctgnnn

SEQ ID NO: 386 - protein, 9 aa, Lactobacillus curvatus
NIPQLTPTP

SEQ ID NO: 387 - DNA, 27 bp, synthetic construct
  misc_feature 1..27: note = Polynucleotide encoding the polypeptide of SEQ ID NO: 386
  source 1..27: mol_type = other DNA
aacattccgcagctgaccccgaccccg

SEQ ID NO: 388 - protein, 18 aa, Bacillus thuringiensis
  VARIANT 4: note = Xaa = any amino acid
  source 1..18: note = subsp. entomocidus
  VARIANT 7: note = Xaa is any amino acid
  VARIANT 10: note = Xaa is any amino acid
DWTXWSXLVXAACSVELL

SEQ ID NO: 389 - DNA, 54 bp, synthetic construct
  misc_feature 1..54: note = Polynucleotide encoding the polypeptide of SEQ ID NO: 388
  misc_feature 10..12: note = nnn = any amino acid-coding triplet
  misc_feature 19..21: note = nnn = any amino acid-coding triplet
  misc_feature 28..30: note = nnn = any amino acid-coding triplet
  source 1..54: mol_type = other DNA
gattggaccnnntggagcnnnctggtgnnngcggcgtgcagcgtggaactgctg

SEQ ID NO: 390 - protein, 30 aa, Lactobacillus curvatus
AYPGNGVHCGKYSCTVDKQTAIGNIGNNAA

SEQ ID NO: 391 - DNA, 90 bp, synthetic construct
  misc_feature 1..90: note = Polynucleotide encoding the polypeptide of SEQ ID NO: 390
  source 1..90: mol_type = other DNA
gcgtatccgggcaacggcgtgcattgcggcaaatatagctgcaccgtggataaacagaccgcgattggcaacattggcaacaacgcggcg

SEQ ID NO: 392 - protein, 43 aa, Carnobacterium divergens
TKYYGNGVYCNSKKCWVDWGTAQGCIDVVIGQLGGGIPGKGKC

SEQ ID NO: 393 - DNA, 129 bp, synthetic construct
  misc_feature 1..129: note = Polynucleotide encoding the polypeptide of SEQ ID NO: 392
  source 1..129: mol_type = other DNA
accaaatattatggcaacggcgtgtattgcaacagcaaaaaatgctgggtggattggggcaccgcgcagggctgcattgatgtggtgattggccagctgggcggcggcattccgggcaaaggcaaatgc

SEQ ID NO: 394 - protein, 62 aa, Enterococcus sp.
NRWYCNSAAGGVGGAAVCGLAGYVGEAKENIAGEVRKGWGMAGGFTHNKACKSFPGSGWASG

SEQ ID NO: 395 - DNA, 186 bp, synthetic construct
  misc_feature 1..186: note = Polynucleotide encoding the polypeptide of SEQ ID NO: 394
  source 1..186: mol_type = other DNA
aaccgctggtattgcaacagcgcggcgggcggcgtgggcggcgcggcggtgtgcggcctggcgggctatgtgggcgaagcgaaagaaaacattgcgggcgaagtgcgcaaaggctggggcatggcgggcggctttacccataacaaagcgtgcaaaagctttccgggcagcggctgggcgagcggc

SEQ ID NO: 396 - protein, 39 aa, Enterococcus faecium
TTKNYGNGVCNSVNWCQCGNVWASCNLATGCAAWLCKLA

SEQ ID NO: 397 - DNA, 117 bp, synthetic construct
  misc_feature 1..117: note = Polynucleotide encoding the polypeptide of SEQ ID NO: 396
  source 1..117: mol_type = other DNA
accaccaaaaactatggcaacggcgtgtgcaacagcgtgaactggtgccagtgcggcaacgtgtgggcgagctgcaacctggcgaccggctgcgcggcgtggctgtgcaaactggcg

SEQ ID NO: 398 - protein, 30 aa, Paenibacillus polymyxa
ASIIKTTIKVSKAVCKTLTCICTGSCSNCK

SEQ ID NO: 399 - DNA, 90 bp, synthetic construct
  misc_feature 1..90: note = Polynucleotide encoding the polypeptide of SEQ ID NO: 398
  source 1..90: mol_type = other DNA
gcgagcattattaaaaccaccattaaagtgagcaaagcggtgtgcaaaaccctgacctgcatttgcaccggcagctgcagcaactgcaaa

SEQ ID NO: 400 - protein, 31 aa, Staphylococcus epidermidis
SASIVKTTIKASKKLCRGFTLTCGCHFTGKK

SEQ ID NO: 401 - DNA, 93 bp, synthetic construct
  misc_feature 1..93: note = Polynucleotide encoding the polypeptide of SEQ ID NO: 400
  source 1..93: mol_type = other DNA
agcgcgagcattgtgaaaaccaccattaaagcgagcaaaaaactgtgccgcggctttaccctgacctgcggctgccattttaccggcaaaaaa

SEQ ID NO: 402 - protein, 43 aa, Enterococcus faecium
KYYGNGVSCNKKGCSVDWGKAIGIIGNNAAANLTTGGKAAWAC

SEQ ID NO: 403 - DNA, 129 bp, synthetic construct
  misc_feature 1..129: note = Polynucleotide encoding the polypeptide of SEQ ID NO: 402
  source 1..129: mol_type = other DNA
aaatattatggcaacggcgtgagctgcaacaaaaaaggctgcagcgtggattggggcaaagcgattggcattattggcaacaacgcggcggcgaacctgaccaccggcggcaaagcggcgtgggcgtgc

SEQ ID NO: 404 - protein, 39 aa, Paenibacillus polymyxa
ATYYGNGLYCNKQKHYTWVDWNKASREIGKITVNGWVQH

SEQ ID NO: 405 - DNA, 117 bp, synthetic construct
  misc_feature 1..117: note = Polynucleotide encoding the polypeptide of SEQ ID NO: 404
  source 1..117: mol_type = other DNA
gcgacctattatggcaacggcctgtattgcaacaaacagaaacattatacctgggtggattggaacaaagcgagccgcgaaattggcaaaattaccgtgaacggctgggtgcagcat

SEQ ID NO: 406 - protein, 35 aa, Bacillus circulans
VNYGNGVSCSKTKCSVNWGIITHQAFRVTSGVASG

SEQ ID NO: 407 - DNA, 105 bp, synthetic construct
  misc_feature 1..105: note = Polynucleotide encoding the polypeptide of SEQ ID NO: 406
  source 1..105: mol_type = other DNA
gtgaactatggcaacggcgtgagctgcagcaaaaccaaatgcagcgtgaactggggcattattacccatcaggcgtttcgcgtgaccagcggcgtggcgagcggc

SEQ ID NO: 408 - protein, 30 aa, Paenibacillus polymyxa
FVYGNGVTSILVQAQFLVNGQRRFFYTPDK

SEQ ID NO: 409 - DNA, 90 bp, synthetic construct
  misc_feature 1..90: note = Polynucleotide encoding the polypeptide of SEQ ID NO: 408
  source 1..90: mol_type = other DNA
tttgtgtatggcaacggcgtgaccagcattctggtgcaggcgcagtttctggtgaacggccagcgccgctttttttataccccggataaa

SEQ ID NO: 410 - protein, 13 aa, Lactobacillus rhamnosus
AVPAVRKTNETLD

SEQ ID NO: 411 - DNA, 39 bp, synthetic construct
  misc_feature 1..39: note = Polynucleotide encoding the polypeptide of SEQ ID NO: 410
  source 1..39: mol_type = other DNA
gcggtgccggcggtgcgcaaaaccaacgaaaccctggat

SEQ ID NO: 412 - protein, 69 aa, Bacillus licheniformis
MKNSAAREAFKGANHPAGMVSEEELKALVGGNDVNPETTPATTSSWTCITAGVTVSASLCPTTKCTSRC

SEQ ID NO: 413 - DNA, 207 bp, synthetic construct
  misc_feature 1..207: note = Polynucleotide encoding the polypeptide of SEQ ID NO: 412
  source 1..207: mol_type = other DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgcccgaccaccaaatgcaccagccgctgc

SEQ ID NO: 414 - protein, 36 aa, Lactobacillus plantarum
KYYGNGLSCSKKGCTVNWGQAFSCGVNRVATAGHGK

SEQ ID NO: 415 - DNA, 108 bp, synthetic construct
  misc_feature 1..108: note = Polynucleotide encoding the polypeptide of SEQ ID NO: 414
  source 1..108: mol_type = other DNA
aaatattatggcaacggcctgagctgcagcaaaaaaggctgcaccgtgaactggggccaggcgtttagctgcggcgtgaaccgcgtggcgaccgcgggccatggcaaa

SEQ ID NO: 416 - protein, 24 aa, Lactobacillus acidophilus
GNPKVAHCASQIGRSTAWGAVSGA

SEQ ID NO: 417 - DNA, 72 bp, synthetic construct
  misc_feature 1..72: note = Polynucleotide encoding the polypeptide of SEQ ID NO: 416
  source 1..72: mol_type = other DNA
ggcaacccgaaagtggcgcattgcgcgagccagattggccgcagcaccgcgtggggcgcggtgagcggcgcg

SEQ ID NO: 418 - protein, 40 aa, Enterococcus faecalis; identical to SEQ ID NO: 1

SEQ ID NO: 419 - DNA, 120 bp, synthetic construct
  misc_feature 1..120: note = Polynucleotide encoding the polypeptide of SEQ ID NO: 418
  source 1..120: mol_type = other DNA
tggctgccgccggcgggcctgctgggccgctgcggccgctggtttcgcccgtggctgctgtggctgcagagcggcgcgcagtataaatggctgggcaacctgtttggcctgggcccgaaa

SEQ ID NO: 420 - protein, 40 aa, Anabaena variabilis
NLDQWLTEQVHEFQDMYLEPQAISNQDITFKLSDLDFIHN

SEQ ID NO: 421 - DNA, 124 bp, Anabaena variabilis
  source 1..124: mol_type = genomic DNA
taatttagatcagtggttaacagaacaagttcatgagtttcaagatatgtacttggaaccacaagcaatatccaatcaagacattaccttcaaactatctgacctagattttattcataattga

SEQ ID NO: 422 - protein, 40 aa, Nostoc sp.; identical to SEQ ID NO: 420

SEQ ID NO: 423 - DNA, 123 bp, Nostoc sp.
  source 1..123: mol_type = genomic DNA
aatttagatcaatggttaacagaacaagttcatgagtttcaagatatgtacttggaaccacaagcaatatccaatcaagacattaccttcaaactgtcagacctagattttattcataattga

SEQ ID NO: 424 - protein, 7 aa, Nostoc azollae
HREKKSA

SEQ ID NO: 425 - DNA, 24 bp, Nostoc azollae
  source 1..24: mol_type = genomic DNA
cacagagagaaaaaatcagcatag

SEQ ID NO: 426 - protein, 23 aa, Acaryochloris marina
TSNNWLAKNYLSMWNKKSSNPNL

SEQ ID NO: 427 - DNA, 72 bp, Acaryochloris marina
  source 1..72: mol_type = genomic DNA
acaagcaataactggctagccaaaaactatctttctatgtggaataaaaagagcagtaatccaaacctttag

SEQ ID NO: 428 - protein, 6 aa, Cyanothece sp.
FRYFWW

SEQ ID NO: 429 - DNA, 21 bp, Cyanothece sp.
  source 1..21: mol_type = genomic DNA
tttagatatttttggtggtaa

SEQ ID NO: 430 - protein, 6 aa, Cyanothece sp.
FRYFWW

SEQ ID NO: 431 - DNA, 21 bp, Cyanothece sp.
  source 1..21: mol_type = genomic DNA
tttagatatttttggtggtaa

SEQ ID NO: 432 - protein, 9 aa, Cyanothece sp.
CGEKWRIFS

SEQ ID NO: 433 - DNA, 27 bp, Cyanothece sp.
  source 1..27: mol_type = genomic DNA
tgtggagaaaaatggagaatttttagc

SEQ ID NO: 434 - protein, 8 aa, Cyanothece sp.
FRLQLWQF

SEQ ID NO: 435 - DNA, 24 bp, Cyanothece sp.
  source 1..24: mol_type = genomic DNA
tttcgcttacaactgtggcaattt

SEQ ID NO: 436 - protein, 16 aa, Cyanothece sp.
LGCNQSSIWSIFFWNH

SEQ ID NO: 437 - DNA, 51 bp, Cyanothece sp.
  source 1..51: mol_type = genomic DNA
ctaggatgtaaccagagcagtatctggtcaatttttttctggaatcattaa

SEQ ID NO: 438 - protein, 40 aa, Microcoleus chthonoplastes
YNLQGLPAIESEDCIPDSVAPSDDWFSGVSSLFNRLTGLG

SEQ ID NO: 439 - DNA, 123 bp, Microcoleus chthonoplastes
  source 1..123: mol_type = genomic DNA
tataacctacaggggttgccagcaattgagtcagaagactgtatcccagattctgtagcgccttcggatgattggttttcaggcgtatcgtctctgtttaaccgcttgactgggttgggttag

SEQ ID NO: 440 - protein, 37 aa, Nostoc sp.
WMAIRRILRCHPFHPGGYDPVPELGEHCCHHDSGNKG

SEQ ID NO: 441 - DNA, 114 bp, Nostoc sp.
  source 1..114: mol_type = genomic DNA
tggatggcgattcgccgcattttgcgttgtcatccattccacccagggggttatgatcctgtaccagagttgggtgagcattgttgtcatcatgatagcgggaataaggggtga

SEQ ID NO: 442 - protein, 35 aa, Anabaena variabilis
WMGIRRILRCHPFHPGGYDPVPEVGEHCCHHDSGK

SEQ ID NO: 443 - DNA, 108 bp, Anabaena variabilis
  source 1..108: mol_type = genomic DNA
tggatggggattcgccgcattttgcgttgtcatccattccacccaggcggttatgatcctgtaccagaggtgggtgagcattgttgtcatcatgatagcgggaagtag

SEQ ID NO: 444 - protein, 48 aa, Nodularia spumigena
WMATRRILRCHPFHPGGYDPVPEVKHNCCDQHLSDSGKQTTEDHHKGS

SEQ ID NO: 445 - DNA, 147 bp, Nodularia spumigena
  source 1..147: mol_type = genomic DNA
tggatggcgactcggcggattttgcgttgtcatcccttccatcctggtggatatgatccagttccagaggtaaaacacaattgctgcgatcagcatctgtccgattctgggaaacagaccacagaagaccatcacaaaggctcgtag

SEQ ID NO: 446 - protein, 34 aa, Nostoc azollae
WMATLRILRCHPFHPGGYDPVPGLAEKSCCDHHD

SEQ ID NO: 447 - DNA, 105 bp, Nostoc azollae
  source 1..105: mol_type = genomic DNA
tggatggcaactttgcggattttacgctgtcatcctttccatcctggtggttatgatcctgtaccaggactagcggaaaaatcctgttgtgaccatcatgattga

SEQ ID NO: 448 - protein, 28 aa, Synechococcus
WLTAKRFCRCHPLHPGGYDPVPEKKSVL

SEQ ID NO: 449 - DNA, 87 bp, Synechococcus
  source 1..87: mol_type = genomic DNA
tggctaacagccaagcgcttttgtcgctgtcatccgcttcatcctggcgggtatgatccggtaccggagaagaaatcggtactctaa

SEQ ID NO: 450 - protein, 23 aa, Prochlorococcus marinus
WLTLRRLSRCHPFTPCGCDPVPD

SEQ ID NO: 451 - DNA, 72 bp, Prochlorococcus marinus
  source 1..72: mol_type = genomic DNA
tggctcaccctgcggcgcctgtctcgttgccatccttttaccccctgtggttgcgacccggtgcctgattaa

SEQ ID NO: 452 - protein, 69 aa, Escherichia coli
MSYKKLYQLTAIFSLPLTILLVSLSSLRIVGEGNSYVDVFLSFIIFLGFIELIHGIRKILVWSGWKNGS

SEQ ID NO: 453 - DNA, 210 bp, Escherichia coli
  source 1..210: mol_type = genomic DNA
atgagttataaaaaactgtaccaattgacggctatatttagtttacctcttactatcttattggtttcactttcatcccttcggattgttggcgaagggaattcttatgttgacgtttttctaagctttataatatttcttggttttattgagctgattcatgggattcgaaagattttggtctggtcaggctggaaaaacggaagttaa

SEQ ID NO: 454 - protein, 85 aa, Escherichia coli
MGLKLDLTWFDKSTEDFKGEEYSKDFGDDGSVMESLGVPFKDNVNNGCFDVIAEWVPLLQPYFNHQIDISDNEYFVSFDYRDGDW

SEQ ID NO: 455 - DNA, 258 bp, Escherichia coli
  source 1..258: mol_type = genomic DNA
atgggacttaaattggatttaacttggtttgataaaagtacagaagattttaagggtgaggagtattcaaaagattttggagatgacggttcagttatggaaagtctaggtgtgccttttaaggataatgttaataacggttgctttgatgttatagctgaatgggtacctttgctacaaccatactttaatcatcaaattgatatttccgataatgagtattttgtttcgtttgattatcgtgatggtgattggtga

SEQ ID NO: 456 - protein, 113 aa, Escherichia coli
MSLRYYIKNILFGLYCTLIYIYLITKNSEGYYFLVSDKMLYAIVISTILCPYSKYAIEYIAFNFIKKDFFERRKNLNNAPVAKLNLFMLYNLLCLVLAIPFGLLGLFISIKNN

SEQ ID NO: 457 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagggtattatttccttgtgtcagataagatgctatatgcaatagtgataagcactattctatgtccatattcaaaatatgctattgaatacatagcttttaacttcataaagaaagattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 458 - protein, 85 aa, Escherichia coli
MGLKLHIHWFDKKTEEFKGGEYSKDFGDDGSVIESLGMPLKDNINNGWFDVEKPWVSILQPHFKNVIDISKFDYFVSFVYRDGNW

SEQ ID NO: 459 - DNA, 258 bp, Escherichia coli
  source 1..258: mol_type = genomic DNA
atggggcttaaattacatattcattggtttgataagaaaaccgaagagtttaaaggcggtgaatactcaaaagacttcggtgatgatggttctgtcattgaaagtctggggatgcctttaaaggataatattaataatggttggtttgatgttgaaaaaccatgggtttcgatattacagccacactttaaaaatgtaatcgatattagtaaatttgattactttgtatcctttgtttaccgggatggtaactggtaa

SEQ ID NO: 460 - protein, 86 aa, Escherichia coli
MELKHSISDYTEAEFLEFVKKICRAEGATEEDDNKLVREFERLTEHPDGSDLIYYPRDDREDSPEGIVKEIKEWRAANGKSGFKQG

SEQ ID NO: 461 - DNA, 261 bp, Escherichia coli
  source 1..261: mol_type = genomic DNA
atggaactgaaacatagtattagtgattataccgaggctgaatttctggagtttgtaaaaaaaatatgtagagctgaaggtgctactgaagaggatgacaataaattagtgagagagtttgagcgattaactgagcacccagatggttcagatctgatttattatcctcgcgatgacagggaagatagtcctgaagggattgtcaaggaaattaaagaatggcgagctgctaacggtaagtcaggatttaaacagggctga

SEQ ID NO: 462 - protein, 178 aa, Citrobacter freundii
MMNEHSIDTDNRKANNALYLFIIIGLIPLLCIFVVYYKTPDALLLRKIATSTENLPSITSSYNPLMTKVMDIYCKTAPFLALILYILTFKIRKLINNTDRNTVLRSCLLSPLVYAAIVYLFCFRNFELTTAGRPVRLMATNDATLLLFYIGLYSIIFFTTYITLFTPVTAFKLLKKRQ

SEQ ID NO: 463 - DNA, 537 bp, Citrobacter freundii
  source 1..537: mol_type = genomic DNA
atgatgaatgaacactcaatagatacggacaacagaaaggccaataacgcattgtatttatttataataatcggattaataccattattgtgcatttttgttgtttactacaaaacgccagacgctttacttttacgtaaaattgctacaagcactgagaatctcccgtcaataacatcctcctacaacccattaatgacaaaggttatggatatttattgtaaaacagcgcctttccttgccttaatactatacatcctaacctttaaaatcagaaaattaatcaacaacaccgacaggaacactgtacttagatcttgtttattaagtccattggtctatgcagcaattgtttatctattctgcttccgaaattttgagttaacaacagccggaaggcctgtcagattaatggccaccaatgacgcaacactattgttattttatattggtctgtactcaataattttctttacaacctatatcacgctattcacaccagtcactgcatttaaattattaaaaaaaaggcagtaa

SEQ ID NO: 464 - protein, 111 aa, Escherichia coli
MNRKYYFNNMWWGWVTGGYMLYMSWDYEFKYRLLFWCISLCGMVLYPVAKWYIEDTALKFTRPDFWNSGFFADTPGKMGLLAVYTGTVFILSLPLSMIYILSVIIKRLSVR

SEQ ID NO: 465 - DNA, 336 bp, Escherichia coli
  source 1..336: mol_type = genomic DNA
atgaacagaaaatattattttaataatatgtggtggggatgggtgacggggggatatatgctgtatatgtcatgggattatgagtttaaatacagattactgttctggtgtatttctctctgcggaatggttttgtatccggttgcaaaatggtatattgaagatacagctctaaaatttacccggcctgatttctggaacagcggtttttttgctgatacacctggaaaaatggggttgcttgcggtttatacgggtactgttttcatattatctcttccgttaagtatgatatatattctttctgttattataaaaaggctgtctgtaagatag

SEQ ID NO: 466 - protein, 115 aa, Escherichia coli
MKLDISVKYLLKSLIPILIILTVFYLGWKDNQENARMFYAFIGCIISAITFPFSMRIIQKMVIRFTGKEFWQKDFFTNPVGGSLTAIFELFCFVISVPVVAIYLIFILCKALSGK

SEQ ID NO: 467 - DNA, 348 bp, Escherichia coli
  source 1..348: mol_type = genomic DNA
atgaaactggatatatctgtaaagtatttactgaaaagcctgataccaatcctcattattcttacagttttttatctgggatggaaagataaccaggaaaatgcaagaatgttttatgcgttcatcggatgcattatcagtgccattacttttcctttttcaatgaggataatacagaaaatggtaataaggtttacagggaaagaattctggcaaaaagacttctttacaaatccagttggcggaagcttaactgcaatatttgaattattctgtttcgttatatcagttcctgtggttgccatttacttaatttttatactctgcaaagccctttcaggaaaatga

SEQ ID NO: 468 - protein, 131 aa, Escherichia coli
MHNTLLEKIIAYLSLPGFHSLNNPPLSEAFNLYVHTAPLAATSLFIFTHKELELKPKSSPLRALKILTPFTILYISMIYCFLLTDTELTLSSKTFVLIVKKRSVFVFFLYNTIYWDIYIHIFVLLVPYRNI

SEQ ID NO: 469 - DNA, 396 bp, Escherichia coli
  source 1..396: mol_type = genomic DNA
atgcacaatacactcctcgaaaaaatcatcgcatacctatccctaccaggatttcattcattaaacaacccgcccctaagcgaagcattcaatctctatgttcatacagcccctttagctgcaaccagcttattcatattcacacacaaagaattagagttaaaaccaaagtcgtcacctctgcgggcactaaagatattaactcctttcactattctttatatatccatgatatactgtttcttgctaactgacacagaactaaccttgtcatcaaaaacatttgtattaatagtcaaaaaacgatctgtttttgtcttttttctatataacactatatattgggatatatatattcacatatttgtacttttggttccttataggaacatataa

SEQ ID NO: 470 - protein, 85 aa, Escherichia coli
MELKNSISDYTETEFKKIIEDIINCEGDEKKQDDNLEHFISVTEHPSGSDLIYYPEGNNDGSPEAVIKEIKEWRAANGKSGFKQG

SEQ ID NO: 471 - DNA, 258 bp, Escherichia coli
  source 1..258: mol_type = genomic DNA
atggaactgaaaaacagcattagtgattacactgaaactgaattcaaaaaaattattgaagacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 472 - protein, 98 aa, Lactococcus lactis
  source 1..98: note = subsp. lactis
MKKKQIEFENELRSMLATALEKDISQEERNALNIAEKALDNSEYLPKIILNLRKALTPLAINRTLNHDLSELYKFITSSKASNKNLGGGLIMSWGRLF

SEQ ID NO: 473 - DNA, 297 bp, Lactococcus lactis
  source 1..297: mol_type = genomic DNA; note = subsp. lactis
atgaaaaaaaaacaaatagaatttgaaaacgagctaagaagtatgttggctaccgcccttgaaaaagacattagtcaagaggaaagaaatgctctgaatattgcagaaaaggcgcttgacaattctgaatatttaccaaaaattattttaaacctcagaaaagccctaactccattagctataaatcgaacacttaaccatgatttatctgaactgtataaattcattacaagttccaaagcatcaaacaaaaatttaggtggtggtttaattatgtcgtggggacgactattctaa

SEQ ID NO: 474 - protein, 98 aa, Lactococcus lactis; identical to SEQ ID NO: 472
  source 1..98: note = subsp. cremoris

SEQ ID NO: 475 - DNA, 297 bp, Lactococcus lactis; identical to SEQ ID NO: 473
  source 1..297: mol_type = genomic DNA; note = subsp. cremoris

SEQ ID NO: 476 - protein, 87 aa, Escherichia coli
MNKMAMIDLAKLFLASKITAIEFSERICVERRRLYGVKDLSPNILNCGEELFMAAERFEPDADRANYEIDDNGLKVEVRSILEKFKL

SEQ ID NO: 477 - DNA, 249 bp, Escherichia coli
  source 1..249: mol_type = genomic DNA
atgatcgatttggcgaaattatttttagcttcgaaaattacagtgattgagttttcagagcgaatttgtgttgaacggagaagattgtatggtgttaaggatttgtctccgaatatattaaattgtggggaagagttgtctatggctgctgagcgatttgagcctgatgcagatagggctaattatgaaattgatgataatggacttaaggtcgaggtccgatctatcttggaaaaacttaaatcataa

SEQ ID NO: 478 - protein, 83 aa, Escherichia coli
MKLSPKAAIEVCNEAAKKGLWILGIDGGHWLNPGFRIDSSASWTYDMPEEYKSKIPENNRLAIENIKDDIENGYTAFIITLKM

SEQ ID NO: 479 - DNA, 243 bp, Escherichia coli
  source 1..243: mol_type = genomic DNA
atgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggagaatacaaatcaaaaatccctgaaaataatagattggctattgaaaatattaaagatgatattgagaatggatacactgctttcattatcacgttaa

SEQ ID NO: 480 - protein, 85 aa, Escherichia coli
MGLKLHINWFDKRTEEFKGGEYSKDFGDDGSVIERLGMPFKDNINNGWFDVIAEWVPLLQPYFNHQIDISDNEYFVSFDYRDGDW

SEQ ID NO: 481 - DNA, 258 bp, Escherichia coli
  source 1..258: mol_type = genomic DNA
atggggcttaaattacatattaattggtttgataagacgaccgaggaatttaaaggtggtgagtattcaaaagattttggagatgatggctcggtcattgaacgtcttggaatgcctttaaaagataatatcaataatggttggtttgatgttatagctgaatgggtacctttgctacaaccatactttaatcatcaaattgatatttccgataatgagtattttgtttcgtttgattatcgtgatggtgattggtga

SEQ ID NO: 482 - protein, 85 aa, Escherichia coli
MELKKSIGDYTETEFKKIIENIINCEGDEKKQDDNLEHFISVTEHPSGSDLIYYPEGNNDGSPEAVIKEIKEWRAANGKSGFKQG

SEQ ID NO: 483 - DNA, 258 bp, Escherichia coli
  source 1..258: mol_type = genomic DNA
gtggagctaaagaaaagtattggtgattacactgaaaccgaattcaaaaaaattattgaaaacatcatcaattgtgaaggtgatgaaaaaaaacaggatgataacctcgagcattttataagtgttactgagcatcctagtggttctgatctgatttattacccagaaggtaataatgatggtagccctgaagctgttattaaagagattaaagaatggcgagctgctaacggtaagtcaggatttaaacagggctga

SEQ ID NO: 484 - protein, 86 aa, Escherichia coli [sequence omitted]

SEQ ID NO: 485 - DNA, 261 bp, Escherichia coli
  source 1..261: mol_type = genomic DNA
atggaactgaagcatagcattagtgattatacagaagctgaatttttacaacttgtaacaacaatttgtaatgcgaacacttccagtgaagaagaactggttaaattggttacacactttgaggaaatgactgagcaccctagtggtagtgatttaatatattacccaaaagaaggtgatgatgactcaccttcaggtattgtaaacacagtaaaacaatggcgagccgctaacggtaagtcaggatttaaacagggctaa

SEQ ID NO: 486 - protein, 141 aa, Escherichia coli
MLTLYGYIRNVFLYRMNDRSCGDFMKVISMKFIFILTIIALAAVFFWSEDKGPACYQVSDEQARTFVKNDYLQRMKRWDNDVQLLGTEIPKITWEKIERSLTDVEDEKTLLVPFKAEGPDGKRMYYGMYHCEEGYVEYAND

SEQ ID NO: 487 - DNA, 354 bp, Escherichia coli
  source 1..354: mol_type = genomic DNA
atgaaagtaattagcatgaaatttatttttattttaacgattattgctcttgctgctgtttttttctggtctgaagataaaggtccggcatgctatcaggtcagcgatgaacaggccagaacgtttgtaaaaaatgattacctgcaaagaatgaaacgctgggacaacgatgtacaacttcttggtacagaaatcccgaaaattacatgggaaaagattgagagaagtttaacagatgttgaagatgaaaaaacacttcttgtcccatttaaagctgaaggcccggacggtaagagaatgtattatggcatgtaccattgtgaggagggatatgttgaatatgcgaatgactaa

SEQ ID NO: 488 - protein, 175 aa, Escherichia coli
MTSNKDKNKKANEILYAFSIIGIIPLMAILILRINDPYSQVLYYLYNKVAFLPSITSLHDPVMTTLMSNYNKTAPVMGILVFLCTYKTREIIKPVTRKLVVQSCFWGPVFYAILIYITLFYNLELTTAGGFFKLLSHNVITLFILYCSIYFTVLTMTYAILLMPLLVIKYFKGRQ

SEQ ID NO: 489 - DNA, 528 bp, Escherichia coli
  source 1..528: mol_type = genomic DNA
atgaccagcaataaagataagaacaagaaagcaaacgaaatattatatgcattttccataatcgggattattccattaatggctatattaatacttcgaataaatgatccatattctcaagtgctgtactacttatataataaggtggcatttctcccttctattacatcattgcatgatcccgtcatgacaacacttatgtcaaactacaacaagacagcgccagttatgggtattctcgtttttctttgcacatataagacaagagaaatcataaagccagtaacaagaaaacttgttgtgcaatcctgtttctgggggcccgttttttatgccattctgatttatatcacactgttctataatctggaactaacaacagcaggtggtttttttaaattattatctcataatgtcatcactctgtttattttatattgctccatttactttactgttttaaccatgacatatgcgattttactgatgccattacttgtcattaaatattttaaagggaggcagtaa

SEQ ID NO: 490 - protein, 78 aa, Escherichia coli
MDRKRTKLELLFAFIINATAIYIALAIYDCVFRGKDFLSMHTFCFSALMSAICYFVGDNYYSISDKIKRRSYENSDSK

SEQ ID NO: 491 - DNA, 237 bp, Escherichia coli
  source 1..237: mol_type = genomic DNA
atggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatga

SEQ ID NO: 492 - protein, 113 aa, Shigella sonnei
MSLRYYIKNILFGLYCALIYIYLITKNNEGYYFLASDKMLYAIVISTILCPYSKYAIEHIFFKFIKKDFFRKRKNLNKCPRGKIKPYLCVYNLLCLVLAIPFGLLGLVYINKE

SEQ ID NO: 493 - DNA, 342 bp, Shigella sonnei
  source 1..342: mol_type = genomic DNA
atgagtttaagatactacataaaaaatattttgtttggcctatactgcgcacttatatatatataccttataacaaaaaacaacgaagggtattatttcctagcgtcagataagatgctatacgcaatagtgataagcactattctatgcccatattcaaaatatgctattgaacacatattttttaagttcataaagaaagattttttcagaaaaagaaaaaacctaaataaatgcccccgtggcaaaattaaaccgtatttatgcgtatacaatctactttgtttggtcctagcaatcccatttggattgctaggacttgtttatatcaataaagaataa

SEQ ID NO: 494 - protein, 113 aa, Escherichia coli
MSLRYYIKNILFGLYCTLIYIYLITKNSEEYYFLVTDKMLYAIVISTILCPYSKYAIEHIAFNFIKKHFFERRKNLNNAPVAKLNLFMLYNLLCLVLAIPFGLLGLFISIKNN

SEQ ID NO: 495 - DNA, 342 bp, Escherichia coli
  source 1..342: mol_type = genomic DNA
atgagcttaagatactacataaaaaatattttatttggcctgtactgcacacttatatatatataccttataacaaaaaacagcgaagagtattatttccttgtgacagataagatgctatatgcaatagtgataagcactattctatgtccatattcaaaatatgctattgaacacatagcttttaacttcataaagaaacattttttcgaaagaagaaaaaacctaaataacgcccccgtagcaaaattaaacctatttatgctatataatctactttgtttggtcctagcaatcccatttggattgctaggactttttatatcaataaagaataattaa

SEQ ID NO: 496 - protein, 113 aa, Leuconostoc gelidum
MRKNNILLDDAKIYTNKLYLLLIDRKDDAGYGDICDVLFQVSKKLDSTKNVEALINRLVNYIRITASTNRIKFSKDEEAVIIELGVIGQKAGLNGQYMADFSDKSQFYSIFER

SEQ ID NO: 497 - DNA, 342 bp, Leuconostoc gelidum
  source 1..342: mol_type = genomic DNA
ttgagaaaaaataacattttattggacgatgctaaaatatacacgaacaaactctatttgctattaatcgatagaaaagatgacgctgggtatggagatatttgtgatgttttgtttcaggtatccaaaaaattagatagcacaaaaaatgtagaagcattgattaaccgattggtcaattatatacgaattaccgcttcaacaaacagaattaagttttcaaaagatgaagaggctgtaattatagaacttggtgtaattggtcagaaggctggattaaacggccaatacatggctgatttttctgacaaatctcagttttatagtatctttgaaagataa

SEQ ID NO: 498 - protein, 91 aa, Lactococcus lactis
  source 1..91: note = subsp. cremoris
MKKKVDTEKQITSWASDLASKNETKVQEKLILSSYIQDIENHVYFPKAMISLEKKLRDQNNICALSKEVNQFYFKVVEVNQRKSWMVGLIV

SEQ ID NO: 499 - DNA, 276 bp, Lactococcus lactis
  source 1..276: mol_type = genomic DNA; note = subsp. cremoris
atgaaaaaaaaagttgatacagaaaaacaaattacttcttgggcatctgacttagcttccaaaaatgaaacaaaggttcaagaaaaattaatactgtcttcttatattcaggacatcgaaaaccatgtttactttccaaaagcaatgatttctttagaaaaaaaattacgagaccaaaataatatttgcgctttatcaaaagaagtcaatcagttttattttaaagttgttgaagtaaatcaaagaaaatcctggatggtaggtttgatagtttaa

SEQ ID NO: 500 - protein, 112 aa, Pediococcus acidilactici
MNKTKSEHIKQQALDLFTRLQFLLQKHDTIEPYQYVLDILETGISKTKHNQQTPERQARVVYNKIASQALVDKLHFTAEENKVLAAINELAHSQKGWGEFNMLDTTNTWPSQ

SEQ ID NO: 501 - DNA, 339 bp, Pediococcus acidilactici
  source 1..339: mol_type = genomic DNA
atgaataagactaagtcggaacatattaaacaacaagctttggacttatttactaggctacagtttttactacagaagcacgatactatcgaaccttaccagtacgttttagatattctggagactggtatcagtaaaactaaacataaccagcaaacgcctgaacgacaagctcgtgtagtctacaacaagattgccagccaagcgttagtagataagttacattttactgccgaagaaaacaaagttctagcagccatcaatgaattggcgcattctcaaaaagggtggggcgagtttaacatgctagatactaccaatacgtggcctagccaatag

SEQ ID NO: 502 - protein, 88 aa, Carnobacterium maltaromaticum
MIKDEKINKIYALVKSALDNTDVKNDKKLSLLLMRIQETSINGELFYDYKKELQPAISMYSIQHNFRVPDDLVKLLALVQTPKAWSGF

SEQ ID NO: 503 - DNA, 267 bp, Carnobacterium maltaromaticum
  source 1..267: mol_type = genomic DNA
atgataaaagatgaaaaaataaataaaatctatgctttagttaagagcgcacttgataatacggatgttaagaatgataaaaaactttctttacttcttatgagaatacaagaaacatcaattaatggagaactattttacgattataaaaaagaattacagccagctattagtatgtactctattcaacataactttcgggttcctgacgatctagtaaaactgttagcattagttcaaacacctaaagcttggtcagggttttaa

SEQ ID NO: 504 - protein, 111 aa, Carnobacterium maltaromaticum
MDIKSQTLYLNLSEAYKDPEVKANEFLSKLVVQCAGKLTASNSENSYIEVISLLSRGISSYYLSHKRIIPSSMLTIYTQIQKDIKNGNIDTEKLRKYEIAKGLMSVPYIYF

SEQ ID NO: 505 - DNA, 336 bp, Carnobacterium maltaromaticum
  source 1..336: mol_type = genomic DNA
atggatataaagtctcaaacattatatttgaatctaagcgaggcatataaagaccctgaagtaaaagctaatgaattcttatcaaaattagttgtacaatgtgctgggaaattaacagcttcaaacagtgagaacagttatattgaagtaatatcattgctatctaggggtatttctagttattatttatcccataaacgtataattccttcaagtatgttaactatatatactcaaatacaaaaggatataaaaaacgggaatattgacaccgaaaaattaaggaaatatgagatagcaaaaggattaatgtccgttccttatatatatttctaa

SEQ ID NO: 506 - protein, 245 aa, Lactococcus lactis
  source 1..245: note = subsp. lactis
MRRYLILIVALIGITGLSGCYQTSHKKVRFDEGSYTNFIYDNKSYFVTDKEIPQENVNNSKVKFYKLLIVDMKSEKLLSSSNKNSVTLVLNNIYEASDKSLCMGINDRYYKILPESDKGAVKALRLQNFDVTSDISDDNFVIDKNDSRKIDYMGNIYSISDTTVSDEELGEYQDVLAEVRVFDSVSGKSIPRSEWGRIDKDGSNSKQSRTEWDYGEIHSIRGKSLTEAFAVEINDDFKLATKVGN

SEQ ID NO: 507 - DNA, 738 bp, Lactococcus lactis
  source 1..738: mol_type = genomic DNA; note = subsp. lactis
atgagaagatatttaatacttattgtggccttaatagggataacaggtttatcagggtgttatcaaacaagtcataaaaaggtgaggtttgacgaaggaagttatactaattttatttatgataataaatcgtatttcgtaactgataaggagattcctcaggagaacgttaacaattccaaagtaaaattttataagctgttgattgttgacatgaaaagtgagaaacttttatcaagtagcaacaaaaatagtgtgactttggtcttaaataatatttatgaggcttctgacaagtcgctatgtatgggtattaacgacagatactataagatacttccagaaagtgataagggggcggtcaaagctttgagattacaaaactttgatgtgacaagcgatatttctgatgataattttgttattgataaaaatgattcacgaaaaattgactatatgggaaatatttacagtatatcggacaccaccgtatctgatgaagaattgggagaatatcaggatgttttagctgaagtacgtgtgtttgattcagttagtggcaaaagtatcccgaggtctgaatgggggagaattgataaggatggttcaaattccaaacagagtaggacggaatgggattatggcgaaatccattctattagaggaaaatctcttactgaagcatttgccgttgagataaatgatgattttaagcttgcaacgaaggtaggaaactag

SEQ ID NO: 508 - protein, 261 aa, Rhizobium leguminosarum
  source 1..261: note = bv. trifolii
MNDEICLTGGGRTTVTRRGGVVYREGGPWSSTVISLLRHLEASGFAEAPSVVGTGFDERGRETLSFIEGEFVHPGPWSEEAFPQFGMMLRRLHDATASFKPPENSMWRDWFGRNLGEGQHVIGHCDTGPWNIVCRSGLPVGLIDWEVAGPVRADIELAQACWLNAQLYDDDIAERVGLGSVTMRAHQVRLLLDGYGLSRKQRGGFVDKLITFAVHDAAEQAKEAAVTPESNDAEPLWAIAWRTRSASWMLHHRQTLEAALA

SEQ ID NO: 509 - DNA, 786 bp, Rhizobium leguminosarum
  source 1..786: mol_type = genomic DNA; note = bv. trifolii
atgaatgatgagatttgcctgacaggtggcggacgaacgactgtcacgcggcgcggcggagtcgtgtatcgcgaaggcggcccgtggtcatcaaccgtcatttcgctcctgcggcatctggaagcctctggcttcgctgaagctccttccgttgtcggcaccggtttcgatgagcgcggccgggagacattatcgtttatcgagggtgagtttgttcacccaggcccttggtcggaggaggcttttccgcaatttggaatgatgttgcggcgactgcacgatgccaccgcctcgttcaaacctcccgaaaactcgatgtggcgcgattggttcgggcgtaacctcggtgagggtcaacacgtaataggacactgcgacacaggcccatggaacattgtttgccggtcaggattgcctgtcgggttgatagattgggaggtggctgggcctgtcagggcggatatcgaattggcccaggcttgttggctgaatgcccagctctacgatgacgacattgcggagagggtcggattaggctctgtgaccatgagagcgcatcaagttcgcctgctgcttgacggctatggtctgtctcggaagcaacgcggcggcttcgtcgacaagctaatcacgttcgcagttcacgatgcggccgagcaggcgaaagaggcggctgtcacgccagagtcgaacgatgcggaaccgctatgggcaattgcctggcgcactagaagtgcctcctggatgctccatcatcggcaaacactggaagcagcgctggcatag

SEQ ID NO: 510 - protein, 436 aa, Bacillus subtilis
MNNIIPIMSLLFKQLYSRQGKKDAIRIAAGLVILAVFEIGLIRQAGIDESVLRKTYIILALLLMNTYMVFLSVTSQWKESYMKLSCLLPISSRSFWLAQSVVLFVDTCLRRTLFFFILPLFLFGNGTLSGAQTLFWLGRFSFFTVYSIIFGVVLSNHFVKKKNLMFLLHAAIFACVCISAALMPAATIPLCAVHILWAVVIDFPVFLQAPPQQGKMHSFMRRSEFSFYKREWNRFISSKAMLLNYAVMAVFSGFFSFQMMNTGIFNQQVIYIVISALLLICSPIALLYSIEKNDRMLLITLPIKRKTMFWAKYRFYSGLLAGGFLLVVMIVGFISGRSISVLTFLQCIELLLAGAYIRLTADEKRPSFSWQTEQQLWSGFSKYRSYLFCLPLFLAILAGTAVSLAVIPIAGLVIVYYLQKQDGGFFDTSKRERLGS

SEQ ID NO: 511 - DNA, 1000 bp, Bacillus subtilis
  source 1..1000: mol_type = genomic DNA
atgaataacataatccctatcatgtctttgctgttcaaacagctttacagccggcaagggaaaaaggacgccatccgcattgccgcaggccttgtcattctggccgtgtttgaaatcgggctgatccgccaggccggcattgatgaatcggtgttgcgcaaaacgtatatcatactcgcgcttcttttgatgaacacatatatggtgtttctttccgtgacatcacaatggaaggaatcttatatgaagctgagctgcctgctgccgatttcttcacggagcttttggctcgcccagagtgtcgttttgtttgtcgatacctgtttgagaagaactttattcttttttattttaccgctgttcttatttggaaacggaacgctgtcaggggcgcaaacattgttttggctcggcaggttttcgttttttaccgtttactccattattttcggagttgtgctaagcaaccacttcgtcaaaaagaagaacttgatgtttctgctgcatgcggcgatattcgcctgtgtatgtatcagcgccgctttgatgccggccgccacgattccgctttgcgcggttcatatcctgtgggcggtggtcattgactttcctgtctttctgcaggcgcctccgcagcagggcaagatgcattcatttatgcggcgatctgaattttcgttttacaaaagagaatggaaccgatttatctcttctaaagcgatgctgttaaattacgcggtaatggcggtattcagcggcttcttttcgttccagatgatgaacaccggcatcttcaatcagcaagtgatttatatcgtgatttccgcgcttttgctcatctgctcgccgatcgcccttttgtattcgattgaaaaaaatgaccggatgctgctcatcacgcttccgatcaagcgaaaaacgatgttttgggcgaaatatcgcttttattcaggcctattggcaggcggatttctccttgtcgtgatgattgtgggtttca

SEQ ID NO: 512 - protein, 239 aa, Bacillus subtilis
MSILDIHDVSVWYERDNVILEQVDLHLEKGAVYGLLGVNGAGKTTLINTLTGVNRNFSGRFTLCGIEAEAGMPQKTSDQLKTHRYFAADYPLLFTEITAKDYVSFVHSLYQKDFSEQQFASLAEAFHFSKYINRRISELSLGNRQKVVLMTGLLLRAPLFILDEPLVGLDVESIEVFYQKMREYCEAGGTILFSSHLLDVVQRFCDYAAILHNKQIQKVIPIGEETDLRREFFEVIGHE

SEQ ID NO: 513 - DNA, 716 bp, Bacillus subtilis
  source 1..716: mol_type = genomic DNA
gcattttggatatacacgatgtatccgtttggtatgaacgggacaacgtcatcttagagcacgtggacttacacttagaaaaaggcgccgtttacggattgcttggggtaaacggtgccggcaaaacaacactgatcaatacgctgacaggagtgaaccgcaattacagcgggggctttacgctgtgcggcattgaagctgaggccggcatgccgcagaaaacatcagatcaactgaagattcaccgttacttcgccgctgattatccgctgctgtttacagaaattacggcgaaggactatgtgtctttcgtccattcgctttatcaaaaggatttttcagagcgacagtttgccagtttggctgaggcctttcatttttcaaaatacatcaacaggagaatctcggagctgtccttggggaacaggcaaaaggttgtgttgatgacaggattattgctgcgggctcccctgtttattttggatgagccgctcgtcggtttggatgtggaatcaatagaggtcttttatcagaaaatgcgggagtactgtgaggaaggcggaaccattttgttttcttcccatctgctcgatgtcgtgcagagattttgtgattttgcggccattctgcacaacaaacagatccaaaaggtcattccgattggggaggagaccgatctgcggcgggaattttttgaggttatcggccatgaataa

SEQ ID NO: 514 - protein, 53 aa, Bacillus subtilis
MSPAQRRILLYILSFIFVIGAVVYFVKSDYLFTLIFIAIAILFGMRARKADSR

SEQ ID NO: 515 - DNA, 162 bp, Bacillus subtilis
  source 1..162: mol_type = genomic DNA
ttgtcaccagcacaaagaagaattttactgtatatcctttcatttatctttgtcatcggcgcagtcgtctattttgtcaaaagcgattatctgtttacgctgattttcattgccattgccattctgttcgggatgcgcgcgcggaaggctgactcgcgatga

SEQ ID NO: 516 - protein, 87 aa, Escherichia coli
MELKNSISDYTEAEFVQLLKEIEKENVAATDDVLDVLLEHFVKITEHPDGTDLIYYPSDNRDDSPEGIVKEIKEWRAANGKPGFKQG

SEQ ID NO: 517 - DNA, 264 bp, Escherichia coli
  source 1..264: mol_type = genomic DNA
atggaactgaaaaatagtattagtgattacacagaggctgagtttgttcaacttcttaaggaaattgaaaaagagaatgttgctgcaactgatgatgtgttagatgtgttactcgaacactttgtaaaaattactgagcatccagatggaacggatctgatttattatcctagtgataatagagacgatagccccgaagggattgtcaaggaaattaaagaatggcgagctgctaacggtaagccaggatttaaacagggctga

SEQ ID NO: 518 - protein, 87 aa, Pseudomonas aeruginosa
MKSKISEYTEKEFLEFVEDIYTNNKKKFPTEESHIQAVLEFKKLTEHPSGSDLLYYPNENREDSPAGVVKEVKEWRASKGLPGFKAG

SEQ ID NO: 519 - DNA, 264 bp, Pseudomonas aeruginosa
  source 1..264: mol_type = genomic DNA
atgaagtccaagatttccgaatatacggaaaaagagtttcttgagtttgttgaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 520 - protein, 87 aa, Pseudomonas aeruginosa
MKSKISEYTEKEFLEFVKDIYTNNKKKFPTEESHIQAVLEFKKLTEHPSGSDLLYYPNENREDSPAGVVKEVKEWRASKGLPGFKAG

SEQ ID NO: 521 - DNA, 264 bp, Pseudomonas aeruginosa
  source 1..264: mol_type = genomic DNA
atgaagtccaagatttccgaatatacggaaaaagagtttcttgagtttgttaaagacatatacacaaacaataagaaaaagttccctaccgaggagtctcatattcaagccgtgcttgaatttaaaaaactaacggaacacccaagcggctcagaccttctttactaccccaacgaaaatagagaagatagcccagctggagttgtaaaggaagttaaagaatggcgtgcttccaaggggcttcctggctttaaggccggttag

SEQ ID NO: 522 - protein, 95 aa, Enterococcus hirae
MDFTKEEKLLNAISKVYNEATIDDYPDLKEKLFLYSKEISEGKSVGEVSMKLSSFLGRYILKHKFGLPKSLIELQEIVSKESQVYRGWASIGIWS

SEQ ID NO: 523 - DNA, 288 bp, Enterococcus hirae
  source 1..288: mol_type = genomic DNA
atggattttactaaagaagaaaaacttttaaatgcaattagtaaagtatacaatgaagcaactatagatgactatcctgacttaaaagaaaagctctttctttattctaaagaaatcagtgagggaaaaagtgttggtgaagttagtatgaaattaagtagttttcttggaagatatattttaaaacataaatttggattacctaaatctttaatagaattacaagaaattgttagtaaggaatctcaagtatatagaggatgggcttctattggtatttggagttaa

SEQ ID NO: 524 - protein, 113 aa, Leuconostoc mesenteroides
MKKKYRYLEDSKNYTSTLYSLLVDNVDKPGYSDICDVLLQVSKKLDNTQSVEALINRLVNYIRITASTYKIIFSKKEEELIIKLGVIGQKAGLNGQYMADFSDKSQFYSVFDQ

SEQ ID NO: 525 - DNA, 342 bp, Leuconostoc mesenteroides
  source 1..342: mol_type = genomic DNA
ttgaaaaaaaagtatcggtatttagaagatagcaaaaattacactagtacactctattctctgttagttgataatgttgacaaacctggatactcagatatttgcgatgttttgcttcaagtttctaagaagttggataatactcaaagtgttgaagcgctaattaatcgattggttaattatattcgtattactgcttcaacatacaaaattattttttcaaaaaaagaagaggaattgattataaaacttggtgttattggacaaaaagctggacttaatggtcagtatatggctgatttttcagacaagtctcagttttacagcgttttcgatcagtaa

SEQ ID NO: 526 - protein, 93 aa, Escherichia coli
MSFLNFAFSPVFFSIMACYFIVWRNKRNEFVCNRLLSIIIISFLICFIYPWLNYKIEVKYYIFEQFYLFCFLSSLVAVVINLIVYFILYRRCI

SEQ ID NO: 527 - DNA, 282 bp, Escherichia coli
  source 1..282: mol_type = genomic DNA
atgagttttcttaattttgcattttctcctgtattcttctccattatggcgtgttatttcattgtatggagaaataaacgaaacgaatttgtctgcaatagattgctatcaattataataatatcttttttgatatgcttcatatatccatggctaaattacaaaatcgaagttaaatattatatatttgaacagttttatcttttttgttttttatcgtcactcgtggctgttgtaataaacctaattgtatactttatattatacaggagatgtatatga

SEQ ID NO: 528 - protein, 96 aa, Escherichia coli
MHLKYYLHNLPESLIPWILILIFNDNDNTPLLFIFISSIHVLLYPYSKLTISRYIKENTKLKKEPWYLCKLSALFYLLMAIPVGLPSFIYYTLKRN

SEQ ID NO: 529 - DNA, 291 bp, Escherichia coli
  source 1..291: mol_type = genomic DNA
atgcatttaaaatactacctacataatttacctgaatcacttataccatggattcttattttaatatttaacgacaatgataacactcctttgttatttatatttatatcatcaatacatgtattgctatatccatactctaaattaaccatatctagatatatcaaagaaaatacaaagttaaaaaaagaaccctggtacttatgcaagttatctgcattgttttatttattaatggcaatcccagtaggattgccaagtttcatatattacactctaaagagaaattaa

SEQ ID NO: 530 - protein, 344 aa, Escherichia coli
MMIQSHPLLAAPLAVGDTIGFFSSSAPATVTAKNRFFRGVEFLQRKGFKLVSGKLTGKTDFYRSGTIKERAQEFNELVYNPDITCIMSTIGGDNSNSLLPFLDYDAIIANPKIIIGYSDTTALLAGIYAKTGLITFYGPALIPSFGEHPPLVDITYESFIKILTRKQSGIYTYTLPEKWSDESINWNENKILRPKKLYKNNCAFYGSGKVEGRVIGGNLNTLTGIWGSEWMPEILNGDILFIEDSRKSIATIERLFSMLKLNRVFDKVSAIILGKHELFDCAGSKRRPYEVLTEVLDGKQIPVLDGFDCSHTHPMLTLPLGVKLAIDFDNKNISITEQYLSTEK

SEQ ID NO: 531 - DNA, 1000 bp, Escherichia coli
  source 1..1000: mol_type = genomic DNA
atgatgatacaatctcatccactactggccgctcccctggcagtaggagatacaattggtttcttttcatcatctgctccggcaacagttactgcaaaaaatcgtttttttcggggagttgagtttcttcagagaaagggatttaagctggtatcagggaagcttaccggtaaaacagatttttatcgttcaggtactattaaagaaagagctcaagaatttaatgagttagtctacaatcctgatattacctgtataatgtcaacgatcggtggagataacagtaattcactactaccgtttctggactatgatgctatcattgcaaaccccaaaattatcataggttactcagatacaactgctttattagcaggaatatatgcaaaaacagggttaataacattctatggaccagctcttattccttcgtttggtgaacatccacctcttgtggatataacatatgaatcatttattaaaatactaacaagaaaacaatcaggaatatatacctacacattacctgaaaagtggagtgatgagagcataaactggaatgaaaacaagatattaaggcctaagaagctatataaaaacaactgtgccttttatggttccggaaaagttgaggggcgtgtaattggaggaaatctaaatactttgacaggtatatgggggagtgaatggatgcctgaaattcttaatggagatatattgtttattgaggacagtcggaaaagcattgcaacaattgaacgattattctctatgctaaagcttaatcgcgtgtttgataaagttagtgcaataatactcgggaaacatgagctttttgattgtgcaggaagtaaacgcagaccatatgaagtattaacagaggtattagatgggaaacagattcctgtactggatggatttgattgttcacatacacatccaatgctaactcttccacttggtgtaaaattagctattgactttgacaacaaaaatatat

SEQ ID NO: 532 - protein, 90 aa, Lactobacillus sakei
MKADYKKINSILTYTSTALKNPKIIKDKDLVVLLTIIQEEAKQNRIFYDYKRKFRPAVTRFTIDNNFEIPDCLVKLLSAVETPKAWSGFS

SEQ ID NO: 533 - DNA, 268 bp, Lactobacillus sakei
  source 1..268: mol_type = genomic DNA
ggcagattataaaaaaataaattcaatactaacttacacatctactgctttaaaaaaccctaaaattataaaagataaagatttagtagtccttctaactattattcaagaagaagccaaacaaaatagaatcttttatgattataaaagaaaatttcgtccagcggttactcgctttacaattgataataattttgagattcctgattgtttggttaaactactgtcagctgttgaaacacctaaggcgtggtctggatttagttag

SEQ ID NO: 534 - protein, 83 aa, Escherichia coli
MKLSPKAAIEVCNEAAKKGLWILGIDGGHWLNPGFRIDSSASWTYDMPEEYKSKTPENNRLAIENIKDDIENGYTAFIITLKM

SEQ ID NO: 535 - DNA, 251 bp, Escherichia coli
  source 1..251: mol_type = genomic DNA
tgaagttatcaccaaaagctgcaatagaagtttgtaatgaagcagcgaaaaaaggcttatggattttgggcattgatggtgggcattggctgaatcctggattcaggatagatagttcagcatcatggacatatgatatgccggaggaatacaaatcaaaaacccctgaaaataatagattggctattgaaaatattaaagatgatattgagaatggatacactgctttcattatcacgttaaagatgtaa

SEQ ID NO: 536 - protein, 436 aa, Bacillus subtilis
MNNIFPIMSLLFKQLYSRQGKKDAIRIAAGLVILAVFEIGLIRQAGIDESVLGKTYIILALLLMNTYMVFLSVTSQWKESYMKLSCLLPISSRSFWLAQSVVLFVDTCLRRTLFFFILPLFLFGNGTLSGAQTLFWLGRFSFFTVYSILFGVMLSNHFVKKKNSMFLLHAAVFAFVCLSAAFMPAVTIPLCAVHMLWAVIIDFPVFLQAPPHQSKMHFFMRRSEFSFYKREWNRFISSKAMLLNYVVMAAFSGFFSFQMMNTGIFNQQVIYIVISALLLICSPIALLYSIEKNDRMLLITLPIKRRTMFWAKYRFYSGLLAGGFLLVAIIVGFISGRPISALTFVQCMELLLAGAFIRLTADEKRPSFGWQTEQQLWSGFSKYRSYLFCLPLFLATLAGTAVSLAVIPIAALIIVYYLQKQDGGFFDTSKRERIGS

SEQ ID NO: 537 - DNA, 1000 bp, Bacillus subtilis
  source 1..1000: mol_type = genomic DNA
ttggggaggagaccgatctgcggcgggaattttttgaggttatcggccatgaataacatattccccatcatgtcgttgctgttcaaacagctgtacagccggcaagggaaaaaggacgctatccgcattgctgcagggcttgtgattctcgccgtgtttgaaatcgggctgatccgacaagccggcattgacgaatcggtgttgggaaaaacgtatatcatattggcgcttctcttaatgaacacgtatatggtgtttctttccgtgacatcacaatggaaggaatcttatatgaagctgagctgtctgctgccgatttcatcacggagcttttggctcgcccagagtgtcgttctgtttgtcgatacctgtttgagaagaacgttattcttttttattttaccgctgttcttatttggaaacggaacgctgtcaggggcgcaaacattgttttggcttggcagattttcgttttttaccgtttactcgattctattcggagttatgctaagcaaccatttcgtcaaaaagaagaactcgatgtttctgctgcatgcggcggtattcgcctttgtatgcctcagtgccgcttttatgccggccgtcacgatcccgctatgcgcggttcacatgctatgggcggtgatcattgactttccggtctttctgcaggcgcctccgcatcagagcaagatgcatttttttatgcggcgatctgaattttcgttttacaaaagagaatggaaccgatttatttcttctaaagcgatgctgttaaattacgtggtgatggcggcgttcagcggattcttttcgttccagatgatgaacactggcatcttcaatcagcaagtgatttatattgtgatttccgctctattgctgatttgctcgccgatcgcccttttgtactctattgaaaaaaacgatcgcatgctgctcatcacgcttccaattaaaagaagaacgatgttttgggcgaaatatcgcttttattcag

SEQ ID NO: 538 - protein, 580 aa, Escherichia coli
MERKQKNSLFNYIYSLMDVRGKFLFFSMLFITSLSSIIISISPLILAKITDLLSGSLSNFSYEYLVLLACLYMFCVISNKASVFLFMILQSSLRINMQKKMSLKYLRELYNENITNLSKNNAGYTTQSLNQASNDIYILVRNVSQNILSPVIQLISTIVVVLSTKDWFSAGVFFLYILVFVIFNTRLTGSLASLRKHSMDITLNSYSLLSDTVDNMIAAKKNNALRLISERYEDALTQENNAQKKYWLLSSKVLLLNSLLAVILFGSVFIYNILGVLNGVVSIGHFIMITSYIILLSTPVENIGALLSEIRQSMSSLAGFIQRHAENKATSPSIPFLNMERKLNLSIRELSFSYSDDKKILNSVSLDLFTGKMYSLTGPSGSGKSTLVKIISGYYKNYFGDIYLNDISLRNISDEDLNDAIYYLTQDDYIFMDTLRFNLRLANYDASENEIFKVLKLANLSVVNNEPVSLDTHLINRGNNYSGGQKQRISLARLFLRKPAIIIIDEATSALDYINESEILSSIRTHFPDALIINISHRINLLECSDCVYVLNEGNIVASGHFRDLMVSNEYISGLASVTE

SEQ ID NO: 539 - DNA, 1000 bp, Escherichia coli
  source 1..1000: mol_type = genomic DNA
atggaaagaaaacagaaaaactcattatttaattatatttattcattaatggatgtaagaggtaaatttttattcttttccatgttattcattacatcattatcatcgataatcatatctatttcaccattgattcttgcaaagattacagatttactgtctggctcattgtcaaattttagttatgaatatctggttttacttgcctgtttatacatgttttgcgttatatctaataaagcaagtgtttttttatttatgatactgcaaagtagtctacgtattaacatgcagaaaaaaatgtcgctaaagtatttgagagaattgtataacgaaaatataactaacttgagtaaaaataatgctggatatacaacgcaaagtcttaaccaggcttcaaatgacatttatattcttgtgagaaatgtttcccagaatatcctgtcacctgttatacaacttatttccactattgttgttgttttatctacgaaggactggttttctgccggtgtgttttttctctatattctggtatttgtaatttttaataccagactgactggcagtttagcgtctctcagaaaacacagcatggatatcactcttaactcttatagtctgttatctgatactgttgataacatgatagcagctaaaaagaataatgcattaagacttatttctgaacgttatgaagatgctctcactcaggaaaacaatgctcagaaaaaatactggttactcagttctaaagttcttttattgaactctttacttgctgtaatattatttggttctgtattcatatataatattttaggtgtgctgaatggtgtagttagtatcggccacttcattatgattacatcatatatcattcttctttcaacgccagtggaaaatataggggcattgctaagtgagatcaggcagtcaatgtctagcctggcaggttttattcaacgtcatgccgagaataaagccacatctccttcaa

SEQ ID NO: 540 - protein, 95 aa, Klebsiella pneumoniae
MTLLSFGFSPVFFSVMAFCIISRSKFYPQRTRNKVIVLILLTFFICFLYPLTKVYLVGSYGIFDKFYLFCFISTLIAIAINVVILTINGAKNERN

SEQ ID NO: 541 - DNA, 288 bp, Klebsiella pneumoniae
  source 1..288: mol_type = genomic DNA
atgacattactttcatttggattttctcctgttttcttttcagtcatggcgttctgtatcatttcacgtagtaaattctatccgcagagaacgcgaaacaaagttattgttctgattttactaactttttttatttgttttttatatccattaacaaaagtgtatctggtgggaagttacggtatatttgacaaattctacctcttttgctttatttctacgttaattgcaatagcaattaacgtagtgatacttacaataaatggagctaagaatgagagaaattag

SEQ ID NO: 542 - RNA, 13 nt, synthetic construct
  misc_feature 1..13: note = Kozak sequence
  source 1..13: mol_type = other RNA
gccgccrccatgg

SEQ ID NO: 543 - skipped

SEQ ID NO: 544 - DNA, 30 bp, synthetic construct
  misc_feature 1..30: note = Lead promoter
  source 1..30: mol_type = other DNA
gaaaaccttgtcaatgaagagcgatctatg

SEQ ID NO: 545 - DNA, 30 bp, synthetic construct
  misc_feature 1..30: note = FecA promoter
  source 1..30: mol_type = other DNA
ttctcgttcgactcatagctgaacacaaca

SEQ ID NO: 546 - DNA, 16 bp, synthetic construct
  misc_feature 1..16: note = Cu-sensitive promoter
  source 1..16: mol_type = other DNA
atgacaaaattgtcat

SEQ ID NO: 547 - DNA, 30 bp, synthetic construct
  misc_feature 1..30: note = Fe promoter
  source 1..30: mol_type = other DNA
accaatgctgggaacggccagggcacctaa

SEQ ID NO: 548 - DNA, 30 bp, synthetic construct
  misc_feature 1..30: note = Fe and UV promoters
  source 1..30: mol_type = other DNA
ctgaaagcgcataccgctatggagggggtt

SEQ ID NO: 549 - DNA, 30 bp, synthetic construct
  misc_feature 1..30: note = PrFe (PI + PII rus operon)
  source 1..30: mol_type = other DNA
tagatatgcctgaaagcgcataccgctatg

SEQ ID NO: 550 - DNA, 30 bp, synthetic construct
  misc_feature 1..30: note = Lux cassette right promoter
  source 1..30: mol_type = other DNA
tgttatagtcgaatacctctggcggtgata

SEQ ID NO: 551 - DNA, 30 bp, synthetic construct
  misc_feature 1..30: note = P(Las) TetO
  source 1..30: mol_type = other DNA
ttttggtacactccctatcagtgatagaga

SEQ ID NO: 552 - DNA, 30 bp, synthetic construct
  misc_feature 1..30: note = P(Las) CIO
  source 1..30: mol_type = other DNA
ctttttggtacactacctctggcggtgata

SEQ ID NO: 553 - DNA, 30 bp, synthetic construct
  misc_feature 1..30: note = P(Rhl)
  source 1..30: mol_type = other DNA
tacgcaagaaaatggtttgttatagtcgaa

SEQ ID NO: 554 - DNA, 30 bp, synthetic construct
  misc_feature 1..30: note = Double Promoter (LuxR/HSL, positive / cI, negative)
  source 1..30: mol_type = other DNA
cgtgcgtgttgataacaccgtgcgtgttga

SEQ ID NO: 555 - DNA, 30 bp, synthetic construct
  misc_feature 1..30: note = P2 promoter in agr operon from S. aureus
  source 1..30: mol_type = other DNA
agattgtactaaatcgtataatgacagtga

SEQ ID NO: 556 - DNA, 30 bp, synthetic construct
  misc_feature 1..30: note = plux-cI hybrid promoter
  source 1..30: mol_type = other DNA
gtgttgatgcttttatcaccgccagtggta

SEQ ID NO: 557 - DNA, 30 bp, synthetic construct
  misc_feature 1..30: note = plux-lac hybrid promoter
  source 1..30: mol_type = other DNA
agtgtgtggaattgtgagcggataacaatt

SEQ ID NO: 558 - DNA, 30 bp, synthetic construct
  misc_feature 1..30: note = CinR, CinL and glucose controlled promotor
  source 1..30: mol_type = other DNA
acatcttaaaagttttagtatcatattcgt

SEQ ID NO: 559 - DNA, 30 bp, synthetic construct; identical to SEQ ID NO: 553
  misc_feature 1..30: note = RhIR promoter repressible by CI
  source 1..30: mol_type = other DNA

SEQ ID NO: 560 - DNA, 30 bp, synthetic construct
  misc_feature 1..30: note = Reverse Lux Promoter
  source 1..30: mol_type = other DNA
tcttgcgtaaacctgtacgatcctacaggt

SEQ ID NO: 561 - DNA, 30 bp, synthetic construct
  misc_feature 1..30: note = rhlI promoter
  source 1..30: mol_type = other DNA
atcctcctttagtcttccccctcatgtgtg

SEQ ID NO: 562 - DNA, 30 bp, synthetic construct
  misc_feature 1..30: note = lasI promoter
  source 1..30: mol_type = other DNA
taaaattatgaaatttgcataaattcttca

SEQ ID NO: 563 - DNA, 30 bp, synthetic construct
  misc_feature 1..30: note = LuxR+3OC6HSL independent R0065
  source 1..30: mol_type = other DNA
gtgttgactattttacctctggcggtgata

SEQ ID NO: 564 - DNA, 30 bp, synthetic construct
  misc_feature 1..30: note = LasR/LasI Inducible & RHLR/RHLI repressible Promoter
  source 1..30: mol_type = other DNA
gaaatctggcagtttttggtacacgaaagc

SEQ ID NO: 565 - DNA, 30 bp, synthetic construct
  misc_feature 1..30: note = pLux/cI Hybrid Promoter
  source 1..30: mol_type = other DNA
acaccgtgcgtgttgatatagtcgaataaa

SEQ ID NO: 566 - DNA, 30 bp, synthetic construct
  misc_feature 1..30: note = pLas promoter
  source 1..30: mol_type = other DNA
aaaattatgaaatttgtataaattcttcag

SEQ ID NO: 567 - DNA, 30 bp, synthetic construct
  misc_feature 1..30: note = pLas/cI Hybrid Promoter
  source 1..30: mol_type = other DNA
ggttctttttggtacctctggcggtgataa

SEQ ID NO: 568 - DNA, 30 bp, synthetic construct
  misc_feature 1..30: note = pLas/Lux Hybrid Promoter
  source 1..30: mol_type = other DNA
tgtaggatcgtacaggtataaattcttcag

SEQ ID NO: 569 - DNA, 30 bp, synthetic construct
  misc_feature 1..30: note = pLux
  source 1..30: mol_type = other DNA
caagaaaatggtttgttatagtcgaataaa

SEQ ID NO: 570 - DNA, 30 bp, synthetic construct
  misc_feature 1..30: note = pLux/Las Hybrid Promoter
  source 1..30: mol_type = other DNA
ctatctcatttgctagtatagtcgaataaa

SEQ ID NO: 571 - DNA, 30 bp, synthetic construct
  misc_feature 1..30: note = Hybrid promoter: HSL-LuxR activated, P22 C2 repressed
  source 1..30: mol_type = other DNA
tagtttataatttaagtgttctttaatttc

SEQ ID NO: 572 - DNA, 30 bp, synthetic construct
  misc_feature 1..30: note = PAI+LasR -> LuxI (AI)
  source 1..30: mol_type = other DNA
caccttcgggtgggcctttctgcgtttata

SEQ ID NO: 573 - DNA, 30 bp, synthetic construct
  misc_feature 1..30: note = PAI+LasR -> LasI & AI+LuxR --| LasI
  source 1..30: mol_type = other DNA
aataactctgatagtgctagtgtagatctc

SEQ ID NO: 574 - DNA, 30 bp, synthetic construct; identical to SEQ ID NO: 572
  misc_feature 1..30: note = PAI+LasR -> LasI+GFP & AI+LuxR --| LasI+GFP
  source 1..30: mol_type = other DNA

SEQ ID NO: 575 - DNA, 30 bp, synthetic construct; identical to SEQ ID NO: 572
  misc_feature 1..30: note = Complex QS -> LuxI & LasI circuit
  source 1..30: mol_type = other DNA

SEQ ID NO: 576 - DNA, 30 bp, synthetic construct; identical to SEQ ID NO: 569
  misc_feature 1..30: note = position 3 mutated promoter lux pR-3 (luxR & HSL regulated)
  source 1..30: mol_type = other DNA

SEQ ID NO: 577 - DNA, 30 bp, synthetic construct; identical to SEQ ID NO: 569
  misc_feature 1..30: note = position 5 mutated promoter lux pR-5 (luxR & HSL regulated)
  source 1..30: mol_type = other DNA

SEQ ID NO: 578 - DNA, 30 bp, synthetic construct; identical to SEQ ID NO: 569
  misc_feature 1..30: note = position 3&5 mutated promoter lux pR-3/5 (luxR & HSL regulated)
  source 1..30: mol_type = other DNA

SEQ ID NO: 579 - DNA, 30 bp, synthetic construct
  misc_feature 1..30: note = Promoter (HSL-mediated luxR repressor)
  source 1..30: mol_type = other DNA
ttgacacctgtaggatcgtacaggtataat

SEQ ID NO: 580 - DNA, 30 bp, synthetic construct; identical to SEQ ID NO: 569
  misc_feature 1..30: note = Promoter (luxR & HSL regulated -- lux pR)
  source 1..30: mol_type = other DNA

SEQ ID NO: 581 - DNA, 30 bp, synthetic construct
  misc_feature 1..30: note = Promoter (luxR & HSL regulated -- lux pL)
  source 1..30: mol_type = other DNA
cacgcaaaacttgcgacaaacaataggtaa

SEQ ID NO: 582 - DNA, 30 bp, synthetic construct
  misc_feature 1..30: note = Promoter (RhlR & C4-HSL regulated)
  source 1..30: mol_type = other DNA
gttagctttcgaattggctaaaaagtgttc

SEQ ID NO: 583 - DNA, 30 bp, synthetic construct
  misc_feature 1..30: note = Promoter (cinR and HSL regulated)
  source 1..30: mol_type = other DNA
ccattctgctttccacgaacttgaaaacgc

SEQ ID NO: 584 - DNA, 30 bp, synthetic construct
  misc_feature 1..30: note = Promoter (LasR & PAI regulated)
  source 1..30: mol_type = other DNA
ggccgcgggttctttttggtacacgaaagc

SEQ ID NO: 585 - DNA, 30 bp, synthetic construct
  misc_feature 1..30: note = Promoter, Standard (luxR and HSL regulated -- lux pR)
  source 1..30: mol_type = other DNA
aagaaaatggtttgttgatactcgaataaa

SEQ ID NO: 586 - DNA, 30 bp, synthetic construct
  misc_feature 1..30: note = P(Bla)
  source 1..30: mol_type = other DNA
gtttatacataggcgagtactctgttatgg

SEQ ID NO: 587 - DNA, 30 bp, synthetic construct
  misc_feature 1..30: note = P(Cat)
  source 1..30: mol_type = other DNA
agaggttccaactttcaccataatgaaaca

SEQ ID NO: 588 - DNA, 30 bp, synthetic construct
  misc_feature 1..30: note = P(Kat)
  source 1..30: mol_type = other DNA
taaacaactaacggacaattctacctaaca

SEQ ID NO: 589 - DNA, 30 bp, synthetic construct
  misc_feature 1..30: note = Template for Building Primer Family Member
  source 1..30: mol_type = other DNA
acatcaagccaaattaaacaggattaacac

SEQ ID NO: 590 - DNA, 30 bp, synthetic construct
  misc_feature 1..30: note = Reverse lambda cI-regulated promoter
  source 1..30: mol_type = other DNA
gaggtaaaatagtcaacacgcacggtgtta

SEQ ID NO: 591 - DNA, 30 bp, synthetic construct
  misc_feature 1..30: note = Key Promoter absorbs 3
  source 1..30: mol_type = other DNA
caggccggaataactccctataatgcgcca

SEQ ID NO: 592 - DNA, 30 bp, synthetic construct
  misc_feature 1..30: note = constitutive promoter family member
  source 1..30: mol_type = other DNA
ggctagctcagtcctaggtacagtgctagc

SEQ ID NO: 593 - DNA, 30 bp, synthetic construct
  misc_feature 1..30: note = constitutive promoter family member
  source 1..30: mol_type = other DNA
agctagctcagtcctaggtattatgctagc

SEQ ID NO: 594 - DNA, 30 bp, synthetic construct
  misc_feature 1..30: note = constitutive promoter family member
  source 1..30: mol_type = other DNA
agctagctcagtcctaggtactgtgctagc

SEQ ID NO: 595 - DNA, 30 bp, synthetic construct
  misc_feature 1..30: note = constitutive promoter family member
  source 1..30: mol_type = other DNA
agctagctcagtcctagggattatgctagc

SEQ ID NO: 596 - DNA, 30 bp, synthetic construct
  misc_feature 1..30: note = constitutive promoter family member
  source 1..30: mol_type = other DNA
agctagctcagtcctaggtattgtgctagc

SEQ ID NO: 597 - DNA, 30 bp, synthetic construct
  misc_feature 1..30: note = constitutive promoter family member
  source 1..30: mol_type = other DNA
ggctagctcagtcctaggtactatgctagc

SEQ ID NO: 598 - DNA, 30 bp, synthetic construct
  misc_feature 1..30: note = constitutive promoter family member
  source 1..30: mol_type = other DNA
ggctagctcagtcctaggtatagtgctagc

SEQ ID NO: 599 - DNA, 30 bp, synthetic construct
  misc_feature 1..30: note = constitutive promoter family member
  source 1..30: mol_type = other DNA
ggctagctcagccctaggtattatgctagc

SEQ ID NO: 600 - DNA, 30 bp, synthetic construct
  misc_feature 1..30: note = constitutive promoter family member
  source 1..30: mol_type = other DNA
agctagctcagtcctaggtataatgctagc

SEQ ID NO: 601 - DNA, 30 bp, synthetic construct
  misc_feature 1..30: note = constitutive promoter family member
  source 1..30: mol_type = other DNA
agctagctcagtcctagggactgtgctagc

SEQ ID NO: 602 - DNA, 30 bp, synthetic construct
  misc_feature 1..30: note = constitutive promoter family member
  source 1..30: mol_type = other DNA
ggctagctcagtcctaggtacaatgctagc

SEQ ID NO: 603 - DNA, 30 bp, synthetic construct; identical to SEQ ID NO: 598
  misc_feature 1..30: note = constitutive promoter family member
  source 1..30: mol_type = other DNA

SEQ ID NO: 604 - DNA, 30 bp, synthetic construct; identical to SEQ ID NO: 595
  misc_feature 1..30: note = constitutive promoter family member
  source 1..30: mol_type = other DNA

SEQ ID NO: 605 - DNA, 30 bp, synthetic construct
  misc_feature 1..30: note = constitutive promoter family member
  source 1..30: mol_type = other DNA
ggctagctcagtcctagggattatgctagc

SEQ ID NO: 606 - DNA, 30 bp, synthetic construct; identical to SEQ ID NO: 602
  misc_feature 1..30: note = constitutive promoter family member
  source 1..30: mol_type = other DNA

SEQ ID NO: 607 - DNA, 30 bp, synthetic construct
  misc_feature 1..30: note = constitutive promoter family member
  source 1..30: mol_type = other DNA
agctagctcagcccttggtacaatgctagc

SEQ ID NO: 608 - DNA, 30 bp, synthetic construct
  misc_feature 1..30: note = constitutive promoter family member
  source 1..30: mol_type = other DNA
agctagctcagtcctagggactatgctagc

SEQ ID NO: 609 - DNA, 30 bp, synthetic construct
  misc_feature 1..30: note = constitutive promoter family member
  source 1..30: mol_type = other DNA
agctagctcagtcctagggattgtgctagc

SEQ ID NO: 610 - DNA, 30 bp, synthetic construct
  misc_feature 1..30: note = constitutive promoter family member
  source 1..30: mol_type = other DNA
ggctagctcagtcctaggtattgtgctagc

SEQ ID NO: 611 - DNA, 30 bp, synthetic construct; identical to SEQ ID NO: 600
  misc_feature 1..30: note = constitutive promoter family member
  source 1..30: mol_type = other DNA

SEQ ID NO: 612 - DNA, 30 bp, synthetic construct
  misc_feature 1..30: note = 1bp mutant from J23107
  source 1..30: mol_type = other DNA
ggctagctcagtcctaggtattatgctagc

SEQ ID NO: 613 - DNA, 30 bp, synthetic construct; identical to SEQ ID NO: 602
  misc_feature 1..30: note = 1bp mutant from J23114
  source 1..30: mol_type = other DNA

SEQ ID NO: 614 - DNA, 30 bp, synthetic construct
  misc_feature 1..30: note = pBAD reverse
  source 1..30: mol_type = other DNA
aaagtgtgacgccgtgcaaataatcaatgt

SEQ ID NO: 615 - DNA, 30 bp, synthetic construct
  misc_feature 1..30: note = NikR promoter, a protein of the ribbon helix-helix family of trancription factors that repress expre
  source 1..30: mol_type = other DNA
gacgaatacttaaaatcgtcatacttattt

SEQ ID NO: 616 - DNA, 30 bp, synthetic construct
  misc_feature 1..30: note = lacq_Promoter
  source 1..30: mol_type = other DNA
aaacctttcgcggtatggcatgatagcgcc

SEQ ID NO: 617 - DNA, 30 bp, synthetic construct
  misc_feature 1..30: note = lacIQ - promoter sequence
  source 1..30: mol_type = other DNA
tgatagcgcccggaagagagtcaattcagg

SEQ ID NO: 618 - DNA, 30 bp, synthetic construct
  misc_feature 1..30: note = E. Coli CreABCD phosphate sensing operon promoter
  source 1..30: mol_type = other DNA
ttatttaccgtgacgaactaattgctcgtg

SEQ ID NO: 619 - DNA, 30 bp, synthetic construct
  misc_feature 1..30: note = GlnRS promoter
  source 1..30: mol_type = other DNA
catacgccgttatacgttgtttacgctttg

SEQ ID NO: 620 - DNA, 30 bp, synthetic construct
  misc_feature 1..30: note = Constitutive weak promoter of lacZ
  source 1..30: mol_type = other DNA
ttatgcttccggctcgtatgttgtgtggac

SEQ ID NO: 621 - DNA, 30 bp, synthetic construct
  misc_feature 1..30: note = Mutated LacZ promoter
  source 1..30: mol_type = other DNA
ttatgcttccggctcgtatggtgtgtggac

SEQ ID NO: 622 - DNA, 30 bp, synthetic construct
  misc_feature 1..30: note = constitutive promoter with (TA)10 between -10 and -35 elements
  source 1..30: mol_type = other DNA
atatatatatatatataatggaagcgtttt

SEQ ID NO: 623 - DNA, 30 bp, synthetic construct; identical to SEQ ID NO: 622
  misc_feature 1..30: note = constitutive promoter with (TA)9 between -10 and -35 elements
  source 1..30: mol_type = other DNA

SEQ ID NO: 624 - DNA, 30 bp, synthetic construct
  misc_feature 1..30: note = constitutive promoter with (C)10 between -10 and -35 elements
  source 1..30: mol_type = other DNA
ccccgaaagcttaagaatataattgtaagc

SEQ ID NO: 625 - DNA, 30 bp, synthetic construct; identical to SEQ ID NO: 624
  misc_feature 1..30: note = constitutive promoter with (C)12 between -10 and -35 elements
  source 1..30: mol_type = other DNA

SEQ ID NO: 626 - DNA, 30 bp, synthetic construct
  misc_feature 1..30: note = optimized (TA) repeat constitutive promoter with 13 bp between -10 and -35 elements
  source 1..30: mol_type = other DNA
tgacaatatatatatatatataatgctagc

SEQ ID NO: 627 - DNA, 30 bp, synthetic construct
  misc_feature 1..30: note = optimized (TA) repeat constitutive promoter with 15 bp between -10 and -35 elements
  source 1..30: mol_type = other DNA
acaatatatatatatatatataatgctagc

SEQ ID NO: 628 - DNA, 30 bp, synthetic construct
  misc_feature 1..30: note = optimized (TA) repeat constitutive promoter with 17 bp between -10 and -35 elements
  source 1..30: mol_type = other DNA
aatatatatatatatatatataatgctagc

SEQ ID NO: 629 - DNA, 30 bp, synthetic construct
  misc_feature 1..30: note = optimized (TA) repeat constitutive promoter with 19 bp between -10 and -35 elements
  source 1..30: mol_type = other DNA
tatatatatatatatatatataatgctagc

SEQ ID NO: 630 - DNA, 30 bp, synthetic construct; identical to SEQ ID NO: 629
  misc_feature 1..30: note = optimized (TA) repeat constitutive promoter with 21 bp between -10 and -35 elements
  source 1..30: mol_type = other DNA

SEQ ID NO: 631 - DNA, 30 bp, synthetic construct
  misc_feature 1..30: note = optimized (A) repeat constitutive promoter with 17 bp between -10 and -35 elements
  source 1..30: mol_type = other DNA
aaaaaaaaaaaaaaaaaatataatgctagc

SEQ ID NO: 632 - DNA, 30 bp, synthetic construct; identical to SEQ ID NO: 631
  misc_feature 1..30: note = optimized (A) repeat constitutive promoter with 18 bp between -10 and -35 elements
  source 1..30: mol_type = other DNA

SEQ ID NO: 633 - DNA, 30 bp, synthetic construct; identical to SEQ ID NO: 572
  misc_feature 1..30: note = J23101:GFP
  source 1..30: mol_type = other DNA

SEQ ID NO: 634 - DNA, 30 bp, synthetic construct; identical to SEQ ID NO: 572
  misc_feature 1..30: note = J23119:IFP
  source 1..30: mol_type = other DNA

SEQ ID NO: 635 - DNA, 30 bp, synthetic construct; identical to SEQ ID NO: 572
  misc_feature 1..30: note = J23119:HO1
  source 1..30: mol_type = other DNA

SEQ ID NO: 636 - DNA, 30 bp, synthetic construct; identical to SEQ ID NO: 572
  misc_feature 1..30: note = Infrared signal reporter (J23119:IFP:J23119:HO1)
  source 1..30: mol_type = other DNA

SEQ ID NO: 637 - DNA, 30 bp, synthetic construct; identical to SEQ ID NO: 592
  misc_feature 1..30: note = Double terminator + constitutive promoter
  source 1..30: mol_type = other DNA

SEQ ID NO: 638 - DNA, 30 bp, synthetic construct
  misc_feature 1..30: note = Double terminator + Constitutive promoter + Strong RBS
  source 1..30: mol_type = other DNA
tgctagctactagagattaaagaggagaaa

SEQ ID NO: 639 - DNA, 30 bp, synthetic construct
  misc_feature 1..30: note = IPTG inducible Lac promoter cassette
  source 1..30: mol_type = other DNA
ttgtgagcggataacaagatactgagcaca

SEQ ID NO: 640 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 641 - DNA, 30 bp, synthetic construct; identical to SEQ ID NO: 639
  misc_feature 1..30: note = IPTG inducible Lac promoter cassette
  source 1..30: mol_type = other DNA

SEQ ID NO: 642 - DNA, 30 bp, synthetic construct
  misc_feature 1..30: note = M13K07 gene I promoter
  source 1..30: mol_type = other DNA
cctgtttttatgttattctctctgtaaagg

SEQ ID NO: 643 - DNA, 30 bp, synthetic construct
  misc_feature 1..30: note = M13K07 gene II promoter
  source 1..30: mol_type = other DNA
aaatatttgcttatacaatcttcctgtttt

SEQ ID NO: 644 - DNA, 30 bp, synthetic construct
  misc_feature 1..30: note = M13K07 gene III promoter
  source 1..30: mol_type = other DNA
gctgataaaccgatacaattaaaggctcct

SEQ ID NO: 645 - DNA, 30 bp, synthetic construct
  misc_feature 1..30: note = M13K07 gene IV promoter
  source 1..30: mol_type = other DNA
ctcttctcagcgtcttaatctaagctatcg

SEQ ID NO: 646 - DNA, 30 bp, synthetic construct
  misc_feature 1..30: note = M13K07 gene V promoter
  source 1..30: mol_type = other DNA
atgagccagttcttaaaatcgcataaggta

SEQ ID NO: 647 - DNA, 30 bp, synthetic construct
  misc_feature 1..30: note = M13K07 gene VI promoter
  source 1..30: mol_type = other DNA
ctattgattgtgacaaaataaacttattcc

SEQ ID NO: 648 - DNA, 30 bp, synthetic construct
  misc_feature 1..30: note = M13K07 gene VIII promoter
  source 1..30: mol_type = other DNA
gtttcgcgcttggtataatcgctgggggtc

SEQ ID NO: 649 - DNA, 30 bp, synthetic construct
  misc_feature 1..30: note = M13110
  source 1..30: mol_type = other DNA
ctttgcttctgactataatagtcagggtaa

SEQ ID NO: 650 - DNA, 30 bp, synthetic construct
  misc_feature 1..30: note = Modified promoter sequence of g3.
  source 1..30: mol_type = other DNA
aaaccgatacaattaaaggctcctgctagc

SEQ ID NO: 651 - DNA, 30 bp, synthetic construct
  misc_feature 1..30: note = Constitutive Promoter I
  source 1..30: mol_type = other DNA
caccacactgatagtgctagtgtagatcac

SEQ ID NO: 652 - DNA, 30 bp, synthetic construct
  misc_feature 1..30: note = Constitutive Promoter II
  source 1..30: mol_type = other DNA
gccggaataactccctataatgcgccacca

SEQ ID NO: 653 - DNA, 30 bp, synthetic construct
  misc_feature 1..30: note = --Specify Parts List--
  source 1..30: mol_type = other DNA
ttgacaagcttttcctcagctccgtaaact

SEQ ID NO: 654 - DNA, 30 bp, synthetic construct
  misc_feature 1..30: note = Full-length stationary phase osmY promoter
  source 1..30: mol_type = other DNA
ggtttcaaaattgtgatctatatttaacaa

SEQ ID NO: 655 - DNA, 30 bp, synthetic construct; identical to SEQ ID NO: 654
  misc_feature 1..30: note = Minimal stationary phase osmY promoter
  source 1..30: mol_type = other DNA

SEQ ID NO: 656 - DNA, 30 bp, synthetic construct
  misc_feature 1..30: note = htpG Heat Shock Promoter
  source 1..30: mol_type = other DNA
tctattccaataaagaaatcttcctgcgtg

SEQ ID NO: 657 - DNA, 30 bp, synthetic construct
  misc_feature 1..30: note = Promoter veg a constitutive promoter for B. subtilis
  source 1..30: mol_type = other DNA
aaaaatgggctcgtgttgtacaataaatgt

SEQ ID NO: 658 - DNA, 30 bp, synthetic construct
  misc_feature 1..30: note = Promoter 43 a constitutive promoter for B. subtilis
  source 1..30: mol_type = other DNA
aaaaaaagcgcgcgattatgtaaaatataa

SEQ ID NO: 659 - DNA, 30 bp, synthetic construct
  misc_feature 1..30: note = Strong constitutive promoter for Bacillus subtilis
  source 1..30: mol_type = other DNA
aattgcagtaggcatgacaaaatggactca

SEQ ID NO: 660 - DNA, 30 bp, synthetic construct
  misc_feature 1..30: note = PliaG
  source 1..30: mol_type = other DNA
caagcttttcctttataatagaatgaatga

SEQ ID NO: 661 - DNA, 30 bp, synthetic construct
  misc_feature 1..30: note = PlepA
  source 1..30: mol_type = other DNA
tctaagctagtgtattttgcgtttaatagt

SEQ ID NO: 662 - DNA, 30 bp, synthetic construct
  misc_feature 1..30: note = Pveg
  source 1..30: mol_type = other DNA
aatgggctcgtgttgtacaataaatgtagt

SEQ ID NO: 663 - DNA, 30 bp, synthetic construct
  misc_feature 1..30: note = Promoter ctc for B. subtilis
  source 1..30: mol_type = other DNA
atccttatcgttatgggtattgtttgtaat

SEQ ID NO: 664 - DNA, 30 bp, synthetic construct
  misc_feature 1..30: note = Promoter gsiB for B. subtilis
  source 1..30: mol_type = other DNA
taaaagaattgtgagcgggaatacaacaac

SEQ ID NO: 665 - DNA, 30 bp, synthetic construct; identical to SEQ ID NO: 658
  misc_feature 1..30: note = Promoter 43 a constitutive promoter for B. subtilis
  source 1..30: mol_type = other DNA

SEQ ID NO: 666 - DNA, 30 bp, synthetic construct
  misc_feature 1..30: note = Pspv2 from Salmonella
  source 1..30: mol_type = other DNA
tacaaaataattcccctgcaaacattatca

SEQ ID NO: 667 - DNA, 30 bp, synthetic construct
  misc_feature 1..30: note = Pspv from Salmonella
  source 1..30: mol_type = other DNA
tacaaaataattcccctgcaaacattatcg

SEQ ID NO: 668 - DNA, 30 bp, synthetic construct
  misc_feature 1..30: note = T7 promoter (strong promoter from T7 bacteriophage)
  source 1..30: mol_type = other DNA
agggaatacaagctacttgttctttttgca

SEQ ID NO: 669 - DNA, 23 bp, synthetic construct
  misc_feature 1..23: note = T7 Promoter
  source 1..23: mol_type = other DNA
taatacgactcactatagggaga

SEQ ID NO: 670 - DNA, 28 bp, synthetic construct
  misc_feature 1..28: note = T7 Promoter
  source 1..28: mol_type = other DNA
gaatttaatacgactcactatagggaga

SEQ ID NO: 671 - DNA, 19 bp, synthetic construct
  misc_feature 1..19: note = T7 consensus -10 and rest
  source 1..19: mol_type = other DNA
taatacgactcactatagg

SEQ ID NO: 672 - DNA, 30 bp, synthetic construct
  misc_feature 1..30: note = overlapping T7 promoter
  source 1..30: mol_type = other DNA
gagtcgtattaatacgactcactatagggg

SEQ ID NO: 673 - DNA, 30 bp, synthetic construct
  misc_feature 1..30: note = more overlapping T7 promoter
  source 1..30: mol_type = other DNA
agtgagtcgtactacgactcactatagggg

SEQ ID NO: 674 - DNA, 30 bp, synthetic construct
  misc_feature 1..30: note = weaken overlapping T7 promoter
  source 1..30: mol_type = other DNA
gagtcgtattaatacgactctctatagggg

SEQ ID NO: 675 - DNA, 23 bp, synthetic construct
  misc_feature 1..23: note = T7 Consensus Promoter Sequence
  source 1..23: mol_type = other DNA
taatacgactcactatagggaga

SEQ ID NO: 676 - DNA, 23 bp, synthetic construct
  misc_feature 1..23: note = T7 RNAP promoter
  source 1..23: mol_type = other DNA
ttatacgactcactatagggaga

SEQ ID NO: 677 - DNA, 23 bp, synthetic construct
  misc_feature 1..23: note = T7 RNAP promoter
  source 1..23: mol_type = other DNA
gaatacgactcactatagggaga

SEQ ID NO: 678 - DNA, 23 bp, synthetic construct
  misc_feature 1..23: note = T7 RNAP promoter
  source 1..23: mol_type = other DNA
taatacgtctcactatagggaga

SEQ ID NO: 679 - DNA, 23 bp, synthetic construct
  misc_feature 1..23: note = T7 RNAP promoter
  source 1..23: mol_type = other DNA
tcatacgactcactatagggaga

SEQ ID NO: 680 - DNA, 30 bp, synthetic construct
  misc_feature 1..30: note = T7 strong promoter
  source 1..30: mol_type = other DNA
taatacgactcactatagggagaccacaac

SEQ ID NO: 681 - DNA, 30 bp, synthetic construct
  misc_feature 1..30: note = T7 weak binding and processivity
  source 1..30: mol_type = other DNA
taattgaactcactaaagggagaccacagc

SEQ ID NO: 682 - DNA, 30 bp, synthetic construct
  misc_feature 1..30: note = T7 weak binding promoter
  source 1..30: mol_type = other DNA
cgaagtaatacgactcactattagggaaga

SEQ ID NO: 683 - DNA, 30 bp, synthetic construct
  misc_feature 1..30: note = pCyc (Medium) Promoter
  source 1..30: mol_type = other DNA
acaaacacaaatacacacactaaattaata

SEQ ID NO: 684 - DNA, 30 bp, synthetic construct
  misc_feature 1..30: note = pAdh (Strong) Promoter
  source 1..30: mol_type = other DNA
ccaagcatacaatcaactatctcatataca

SEQ ID NO: 685 - DNA, 30 bp, synthetic construct
  misc_feature 1..30: note = pSte5 (Weak) Promoter
  source 1..30: mol_type = other DNA
gatacaggatacagcggaaacaacttttaa

SEQ ID NO: 686 - DNA, 30 bp, synthetic construct
  misc_feature 1..30: note = yeast ADH1 promoter
  source 1..30: mol_type = other DNA
tttcaagctataccaagcatacaatcaact

SEQ ID NO: 687 - DNA, 30 bp, synthetic construct
  misc_feature 1..30: note = cyc100 minimal promoter
  source 1..30: mol_type = other DNA
cctttgcagcataaattactatacttctat

SEQ ID NO: 688 - DNA, 30 bp, synthetic construct; identical to SEQ ID NO: 687
  misc_feature 1..30: note = cyc70 minimal promoter
  source 1..30: mol_type = other DNA

SEQ ID NO: 689 - DNA, 30 bp, synthetic construct; identical to SEQ ID NO: 687
  misc_feature 1..30: note = cyc43 minimal promoter
  source 1..30: mol_type = other DNA

SEQ ID NO: 690 - DNA, 30 bp, synthetic construct; identical to SEQ ID NO: 687
  misc_feature 1..30: note = cyc28 minimal promoter
  source 1..30: mol_type = other DNA

SEQ ID NO: 691 - DNA, 30 bp, synthetic construct; identical to SEQ ID NO: 687
  misc_feature 1..30: note = cyc16 minimal promoter
  source 1..30: mol_type = other DNA

SEQ ID NO: 692 - DNA, 30 bp, synthetic construct
  misc_feature 1..30: note = pPGK1
  source 1..30: mol_type = other DNA
ttatctactttttacaacaaatataaaaca

SEQ ID NO: 693 - DNA, 30 bp, synthetic construct; identical to SEQ ID NO: 683
  misc_feature 1..30: note = pCYC Yeast Promoter
  source 1..30: mol_type = other DNA

SEQ ID NO: 694 - DNA, 30 bp, synthetic construct
  misc_feature 1..30: note = Yeast GPD (TDH3) Promoter
  source 1..30: mol_type = other DNA
gtttcgaataaacacacataaacaaacaaa

SEQ ID NO: 695 - DNA, 30 bp, synthetic construct; identical to SEQ ID NO: 684
  misc_feature 1..30: note = yeast mid-length ADH1 promoter
  source 1..30: mol_type = other DNA

SEQ ID NO: 696 - DNA, 30 bp, synthetic construct
  misc_feature 1..30: note = Yeast CLB1 promoter region, G2/M cell cycle specific
  source 1..30: mol_type = other DNA
accatcaaaggaagctttaatcttctcata

SEQ ID NO: 697 - DNA, 30 bp, synthetic construct
  misc_feature 1..30: note = CMV promoter
  source 1..30: mol_type = other DNA
agaacccactgcttactggcttatcgaaat

SEQ ID NO: 698 - DNA, 30 bp, synthetic construct
  misc_feature 1..30: note = Ubc Promoter
  source 1..30: mol_type = other DNA
ggccgtttttggcttttttgttagacgaag

SEQ ID NO: 699 - protein, 9 aa, Lactobacillus curvatus
NIPQLTPTP

SEQ ID NO: 700 - DNA, 27 bp, Lactobacillus curvatus
  source 1..27: mol_type = genomic DNA
aacattccgcagctgaccccgaccccg

SEQ ID NO: 701 - protein, 18 aa, Bacillus thuringiensis
  VARIANT 4: note = Xaa = any amino acid
  VARIANT 7: note = Xaa is any amino acid
  VARIANT 10: note = Xaa is any amino acid
DWTXWSXLVXAACSVELL

SEQ ID NO: 702 - DNA, 54 bp, Bacillus thuringiensis; identical to SEQ ID NO: 389
  misc_difference 10..12: note = n is A, T, C, or G
  source 1..54: mol_type = genomic DNA
  misc_difference 19..21: note = n is A, T, C, or G
  misc_difference 28..30: note = n is A, T, C, or G

SEQ ID NO: 703 - protein, 30 aa, Lactobacillus curvatus; identical to SEQ ID NO: 390
  source 1..30: note = L442

SEQ ID NO: 704 - DNA, 90 bp, Lactobacillus curvatus; identical to SEQ ID NO: 391
  source 1..90: mol_type = genomic DNA; strain = L442

SEQ ID NO: 705 - protein, 43 aa, Carnobacterium divergens; identical to SEQ ID NO: 392

SEQ ID NO: 706 - DNA, 129 bp, Carnobacterium divergens; identical to SEQ ID NO: 393
  source 1..129: mol_type = genomic DNA

SEQ ID NO: 707 - protein, 24 aa, Microbispora sp.
  source 1..24: note = strain 107891
VTSWSLCTPGCTSPGGGSNCSFCC

SEQ ID NO: 708 - DNA, 72 bp, Microbispora sp.
  source 1..72: mol_type = genomic DNA; strain = 107891
gtgaccagctggagcctgtgcaccccgggctgcaccagcccgggcggcggcagcaactgcagcttttgctgc

SEQ ID NO: 709 - protein, 62 aa, Enterococcus sp.; identical to SEQ ID NO: 394

SEQ ID NO: 710 - DNA, 186 bp, Enterococcus sp.; identical to SEQ ID NO: 395
  source 1..186: mol_type = genomic DNA

SEQ ID NO: 711 - protein, 39 aa, Enterococcus faecium; identical to SEQ ID NO: 396

SEQ ID NO: 712 - DNA, 117 bp, Enterococcus faecium; identical to SEQ ID NO: 397
  source 1..117: mol_type = genomic DNA

SEQ ID NO: 713 - protein, 30 aa, Paenibacillus polymyxa; identical to SEQ ID NO: 398

SEQ ID NO: 714 - DNA, 90 bp, Paenibacillus polymyxa; identical to SEQ ID NO: 399
  source 1..90: mol_type = genomic DNA

SEQ ID NO: 715 - protein, 31 aa, Staphylococcus epidermidis; identical to SEQ ID NO: 400

SEQ ID NO: 716 - DNA, 93 bp, Staphylococcus epidermidis; identical to SEQ ID NO: 401
  source 1..93: mol_type = genomic DNA

SEQ ID NO: 717 - protein, 58 aa, Enterococcus faecium
MEKLTVKEMSQVVGGKYYGNGVSCNKKGCSVDWGKAIGIIGNNAAANLTTGGKAGWKG

SEQ ID NO: 718 - DNA, 178 bp, Enterococcus faecium
  source 1..178: mol_type = genomic DNA
atggaaaaattaactgtgaaagaaatgtcgcaagtagttggcggaaagtactatggtaacggagtatcatgtaataaaaagggatgtagtgttgattggggaaaagctattggtattattggaaataatgctgctgctaatttaactactggcggaaaagcagggtggaaaggttaac

SEQ ID NO: 719 - protein, 39 aa, Paenibacillus polymyxa; identical to SEQ ID NO: 404

SEQ ID NO: 720 - DNA, 93 bp, Paenibacillus polymyxa; identical to SEQ ID NO: 401
  source 1..93: mol_type = genomic DNA

SEQ ID NO: 721 - protein, 35 aa, Bacillus circulans; identical to SEQ ID NO: 406

SEQ ID NO: 722 - DNA, 105 bp, Bacillus circulans; identical to SEQ ID NO: 407
  source 1..105: mol_type = genomic DNA

SEQ ID NO: 723 - protein, 30 aa, Paenibacillus polymyxa; identical to SEQ ID NO: 408

SEQ ID NO: 724 - DNA, 90 bp, Paenibacillus polymyxa; identical to SEQ ID NO: 409
  source 1..90: mol_type = genomic DNA

SEQ ID NO: 725 - protein, 13 aa, Lactobacillus rhamnosus
AVPAVRKTNETLD

SEQ ID NO: 726 - DNA, 39 bp, Lactobacillus rhamnosus; identical to SEQ ID NO: 411
  source 1..39: mol_type = genomic DNA

SEQ ID NO: 727 - protein, 60 aa, Bacillus licheniformis
MKNSAAREAFKGANHPAGMVSEEELKALVGGNDVNPETTPATTSSWTCITAGVTVSASLC

SEQ ID NO: 728 - DNA, 180 bp, Bacillus licheniformis
  source 1..180: mol_type = genomic DNA
atgaaaaacagcgcggcgcgcgaagcgtttaaaggcgcgaaccatccggcgggcatggtgagcgaagaagaactgaaagcgctggtgggcggcaacgatgtgaacccggaaaccaccccggcgaccaccagcagctggacctgcattaccgcgggcgtgaccgtgagcgcgagcctgtgc

SEQ ID NO: 729 - protein, 689 aa, Pseudomonas aeruginosa; identical to SEQ ID NO: 276

SEQ ID NO: 730 - DNA, 2070 bp, Pseudomonas aeruginosa; identical to SEQ ID NO: 277
  source 1..2070: mol_type = genomic DNA

SEQ ID NO: 731 - protein, 36 aa, Lactobacillus plantarum; identical to SEQ ID NO: 414

SEQ ID NO: 732 - DNA, 108 bp, Lactobacillus plantarum; identical to SEQ ID NO: 415
  source 1..108: mol_type = genomic DNA

SEQ ID NO: 733 - protein, 30 aa, Lactococcus lactis
MKTILRFVAGYDIASHKKKTGGYPWERGKA

SEQ ID NO: 734 - DNA, 93 bp, Lactococcus lactis
  source 1..93: mol_type = genomic DNA
atgaaaacaatcctacgttttgttgctggctacgatattgctagtcataaaaagaaaactggcggctatccatgggaacgtggaaaagcttaa

SEQ ID NO: 735 - protein, 24 aa, Lactobacillus acidophilus
GNPKVAHCASQIGRSTAWGAVSGA

SEQ ID NO: 736 - DNA, 72 bp, Lactobacillus acidophilus; identical to SEQ ID NO: 417
  source 1..72: mol_type = genomic DNA

SEQ ID NO: 737 - protein, 53 aa, Lactobacillus salivarius
  source 1..53: note = cp400
MFFNFMKKVDVKKNFGYKEVSRKDLAKVNGGKRKKHRCRVYNNGMPTGMYRWC

SEQ ID NO: 738 - DNA, 162 bp, Lactobacillus salivarius
  source 1..162: mol_type = genomic DNA; strain = cp400
atgttttttaattttatgaaaaaagtagatgtgaagaagaattttggatataaagaagtttctagaaaagatctagctaaagtaaatggtggaaagagaaagaaacatcgttgcagagtttataataatggaatgcctacaggaatgtatcgttggtgctaa

SEQ ID NO: 739 - protein, 5 aa, synthetic construct
  REGION 1..5: note = Caspase 2 consensus sequence
DVADL

SEQ ID NO: 740 - protein, 5 aa, synthetic construct
  REGION 1..5: note = Caspase 2 consensus sequence
DVADI

SEQ ID NO: 741 - protein, 12 aa, synthetic construct
  REGION 1..12: note = Synthetic RAP-binding peptide, RBP
FHWWQTSPAHFS

SEQ ID NO: 742 - protein, 12 aa, synthetic construct
  REGION 1..12: note = Synthetic RAP-binding peptide, RBP
WPFAHWPWQYPR

SEQ ID NO: 743 - protein, 9 aa, synthetic construct
  REGION 1..9: note = Synthetic AgrC ligand
  CROSSLNK 5..9: note = Thiolacton linkage between C5 and F9
GDSVCASYF

SEQ ID NO: 744 - protein, 7 aa, synthetic construct
  REGION 1..7: note = Synthetic AgrC ligand
  CROSSLNK 3..7: note = Thiolacton linkage between C3 and F7
SVCASYF

SEQ ID NO: 745 - protein, 5 aa, synthetic construct
  REGION 1..5: note = Synthetic Cry1Aa ligand
SKADT

SEQ ID NO: 746 - protein, 5 aa, synthetic construct
  REGION 1..5: note = Synthetic Cry1Aa ligand
SKPAD

SEQ ID NO: 747 - protein, 11 aa, synthetic construct
  REGION 1..11: note = Synthetic Fsr ligand
  CROSSLNK 3..11: note = Lacton linkage between S3 and A11
QNSAAAFAAWA

SEQ ID NO: 748 - protein, 11 aa, synthetic construct
  REGION 1..11: note = Synthetic Fsr ligand
  CROSSLNK 3..11: note = Lacton linkage between S3 and A11
QNSAAAFGQWA

SEQ ID NO: 749 - protein, 7 aa, synthetic construct
  REGION 1..7: note = Synthetic AgrC1, AgrC2
  CROSSLNK 4..7: note = Thiolacton linkage between C4 and M7
YSTCFIM

SEQ ID NO: 750 - protein, 7 aa, synthetic construct
  REGION 1..7: note = Synthetic AgrC1, AgrC2
  CROSSLNK 3..7: note = Thiolacton linkage between C3 and M7
STCAFIM

(19) United States
(12) Patent Application Publication
(10) Pub. No.: US 2025/0290112 A1
(43) Pub. Date: Sep. 18, 2025

(54) ENGINEERING ANTIMICROBIAL PEPTIDES

(71) Applicant: Syngulon SA, Seraing (BE)

(72) Inventor: Philippe Gabant, Ottignies Louvain-La-Neuve (BE)

(21) Appl. No.: 18/957,469

(22) Filed: Nov. 22, 2024

Related U.S. Application Data
(62) Division of application No. 16/972,553, filed on Dec. 4, 2020, now Pat. No. 12,173,343.
(60) Provisional application No. 62/681,529, filed on Jun. 6, 2018.

(51) Int. Cl. C12P 21/02; C07K 14/195

(52) U.S. Cl. CPC C12P 21/02; C07K 14/195

(57) ABSTRACT

Embodiments herein relate to methods, systems and kits for engineering antimicrobial peptides such as bacteriocins, for example to have a desired range of activity in a desired range of culture conditions. The antimicrobial peptides may be engineered to have a particular activity for a particular culture, environmental conditions or a range of conditions. Some embodiments include screening an antimicrobial peptides or several candidate antimicrobial peptides for a desired activity. Some embodiments include an iterative process for engineering antimicrobial peptides such as bacteriocins. In some embodiments, the process is performed by automated machine learning.

### REFERENCE TO RELATED APPLICATIONS

The present application is a divisional application of U.S. application Ser. No. 16/972,553, filed Dec. 4, 2020, now U.S. Pat. No. 12,173,343, issued Dec. 24, 2024, which is a U.S. national phase application under 35 U.S.C. § 371 of International Application No. PCT/US2019/035666, filed Jun. 5, 2019, which claims the benefit of U.S. Provisional Application No. 62/681,529, filed Jun. 6, 2018. Each of the aforementioned related applications is incorporated by reference herein in its entirety.

### REFERENCE TO SEQUENCE LISTING, TABLE, OR COMPUTER PROGRAM LISTING

The present application is being filed along with a Sequence Listing in electronic format. The Sequence Listing is provided as a file entitled SYNG005D1_REPLACEMENT_SEQLIST.xml, created and last saved on Jun. 4, 2025, which is 868,492 bytes in size. The information in the electronic format of the Sequence Listing is incorporated herein by reference in its entirety.

### BACKGROUND

Microbial organisms such as bacteria can affect human and animal health, and participate in microbiota associated with a variety of animal organs and tissues. Microbial organism-mediated processes can be used in a variety of industrial processes for the manufacture of products of interest, for example for fermentation in a feedstock. Additionally, microbial organisms can be used to manufacture products in sterile environments, such as in the manufacture of pharmaceuticals, biologics, and cosmetics.

Tuning populations of microbial organisms, for example to reduce or eliminate undesired microbial organisms can be useful for maintaining the industrial processes and maintaining the health of tissues that comprise microbial organisms. Antimicrobial peptides such as bacteriocins can affect the growth or viability of microbial organisms.

### Field

Embodiments herein relate to methods, systems and kits for engineering antimicrobial peptides such as bacteriocins, for example to have a desired range of activity in a desired range of culture conditions.

### SUMMARY

Some embodiments include a method of engineering an antimicrobial peptide, such as a bacteriocin. The method can comprise (a) translating a candidate nucleic acid encoding a candidate antimicrobial peptide (e.g., bacteriocin) in vitro in a translation solution, so that the translation solution comprises the candidate antimicrobial peptide. The method can further comprise (b) combining the candidate antimicrobial peptide and a microbial organism in a solution environment. The method can further comprise (c) culturing the microbial organism and the candidate antimicrobial peptide in the solution environment under selected culture conditions. The method can further comprise (d) detecting inhibition of growth and/or reproduction, or a lack thereof, of the microbial organism in the solution environment. The method can further comprise (e) selecting the candidate nucleic acid upon detection of inhibition of growth and/or reproduction of the microbial organism in the solution environment under the selected culture conditions. The method can further comprise (f) producing a variant nucleic acid of the selected candidate nucleic acid, the variant nucleic acid encoding a variant of the candidate antimicrobial peptide. The method can further comprise repeating (a)-(f) using one or more successive variant nucleic acids as the candidate nucleic acid, until a predetermined level of inhibition of growth and/or reproduction of the microbial organism in the solution environment under the selected culture conditions is achieved. Thus, the antimicrobial peptide has been engineered. In some embodiments, the method further comprises obtaining a sequence of the selected candidate nucleic acid. The method can further comprise indexing the sequence to the detected inhibition of growth and/or reproduction of the microbial organism in the solution environment of (d), wherein (f) comprises producing the variant nucleic acid based on the indexed sequence of the candidate nucleic acid. By way of example, the candidate nucleic acid can be amplified (such as by PCR), and one or more of the amplicons can be sequenced. In some embodiments, the method further comprises obtaining a sequence of an other candidate nucleic acid for which was detected a lack of inhibition of growth and/or reproduction of the microbial organism in another solution environment under the selected culture conditions. The method can further comprise indexing the sequence of the other candidate nucleic acid to the lack of inhibition of growth and/or reproduction of the microbial organism, wherein (f) comprises producing the variant nucleic acid of the selected candidate nucleic acid based on the indexed sequence of the other candidate nucleic acid. In some embodiments, the sequence of the other candidate nucleic acid is obtained if the other candidate nucleic acid produces a lower level of inhibition of growth and/or reproduction of the microbial organism in the solution environment under the selected culture conditions than the candidate nucleic acid. In some embodiments, producing the variant nucleic acid sequence based on the indexed sequence information comprises machine learning, such as automated machine learning. In some embodiments, the method further comprises confirming the inhibition of growth and/or reproduction of the microbial organism in the solution environment by the candidate antimicrobial peptide after (e), said confirming comprising repeating (a)-(d) for the candidate nucleic acid or a copy thereof, wherein the inhibition of growth and/or reproduction is confirmed when, in the repeat of (d), the quantity of microbial organism in the solution environment indicates inhibition of growth and/or reproduction of the microbial organism in the solution environment under the selected culture conditions. In some embodiments, detecting inhibition of growth and/or reproduction, or a lack thereof, of the microbial organism comprises quantifying the microbial organism in the solution environment, in which a decrease in a quantity of the microbial organism in the solution environment over a period of time indicates inhibition of growth and/or reproduction of the microbial organism. In some embodiments, the predetermined level of inhibition of growth and/or reproduction of the microbial organism in the solution environment under the selected culture conditions is a greater level of inhibition than that of a reference naturally-occurring or engineered antimicrobial peptide. In some embodiments, the engineered antimicrobial peptide and the candidate antimicrobial peptide of an earlier iteration of the method each have a potency. The potency of the engineered antimicrobial peptide can be greater than the potency of the candidate antimicrobial peptide. In some embodiments, the engineered antimicrobial peptide has a greater potency than the candidate antimicrobial peptide across a range of culture conditions and/or against a range of microbial organism strains and/or species. In some embodiments, repeating (b) of (a)-(f) comprises combining the translation solution with a different strain or species of microbial organism than that of a previous iteration of (b). In some embodiments, repeating (b) of (a)-(f) comprises combining the translation solution with the same species or strain of microbial organism as a previous iteration of (b). In some embodiments, repeating (c) of (a)-(f) comprises culturing the microbial organism in a different culture environment than a previous iteration of (c). In some embodiments, repeating (c) of (a)-(f) comprises culturing the microbial organism in the same culture environment as a previous iteration of (c). In some embodiments, the candidate nucleic acid comprises DNA. The method can further comprise transcribing the candidate nucleic acid. In some embodiments, the translation solution further comprises a transcription solution, so that the translation solution is configured for transcription and translation of the candidate nucleic acid. In some embodiments, the translation solution comprises a translation reagent, such as a ribosome. In some embodiments, the translation solution comprises one or more post-translational modification enzymes. In some embodiments, the translation solution comprises no more than one candidate nucleic acid sequence encoding a candidate antimicrobial peptide. In some embodiments, the candidate nucleic acid encodes two or more different candidate antimicrobial peptides, so that the solution environment comprises two or more candidate antimicrobial peptides (for example, a cocktail of antimicrobial peptides). The variant nucleic acid can encode variants of at least one of the two or more candidate antimicrobial peptides. Thus, two or more antimicrobial peptides can be co-engineered to inhibit growth and/or reproduction of the microbial organism under the selected culture conditions. In some embodiments, the candidate antimicrobial peptide comprises a chimeric protein. In some embodiments, the selected culture conditions comprise conditions of an industrial process, a pharmaceutical manufacturing process, or a mammalian microbiota. In some embodiments, the mammalian microbiota is of a human gastrointestinal tract, skin, mammary gland, placenta, tissue, biofluid, seminal fluid, uterus, vagina, ovarian follicle, lung, saliva, oral cavity, mucosa, conjunctiva, or biliary tract. In some embodiments, the solution environment comprises two or more species of microbial organism. In some embodiments, the translation solution further comprises a substrate. The candidate nucleic acid can be immobilized on the substrate. The method can further comprise producing the variant nucleic acid immobilized on another substrate that is the same as or different from the substrate. In some embodiments, the substrate comprises a bead, nanoparticle, well, membrane, nitrocellulose, PVDF, nylon, acetate derivative, matrix, pore, plastic, metal, glass, polymer, polysaccharide, or paramagnetic compound. In some embodiments, said producing the variant nucleic acid comprises one or more of amplifying the selected candidate nucleic acid with a degenerate polymerase; amplifying the selected candidate nucleic acid in the presence of a degenerate primer; amplifying the selected candidate nucleic acid in the presence of a degenerate nucleotide;

and mutagenesis reaction on the selected candidate nucleic acid. In some embodiments, a library of candidate nucleic acids is screened. In some embodiments, producing the variant nucleic acid comprises producing a library of variant nucleic acids, the method further comprising performing (a)-(e) on the library of variant nucleic acids. In some embodiments, the method is performed in a microfluidic system. In some embodiments, the translation solution and/or the solution environment is microliter-scale. In some embodiments, the translation solution and/or the solution environment has a volume of 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl. In some embodiments, the solution environment comprises a microdrop. In some embodiments, the candidate antimicrobial peptide and the microbial organism in (b) are each in a microdrop, and said combining comprises combining microdrops into a solution environment comprising a microdrop. In some embodiments, said culturing in (c) comprises culturing a microdrop comprising the microbial organism and the candidate antimicrobial peptide in the solution environment. In some embodiments, a microdrop comprises a nanometer-scale droplet of solution. In some embodiments, the microbial organism comprises, consists essentially of, or consists of a bacteria. For example, the microbial organism can be a bacteria that is resistant to antibiotics, such as Methicillin-resistant Staphylococcus aureus (MRSA). In the method of some embodiments, the antimicrobial peptide comprises, consists essentially of, or consists of a bacteriocin. In some embodiments, the solution environment does not comprise any cells comprising nucleic acid encoding the antimicrobial peptide.

Some embodiments include a microfluidic system for engineering an antimicrobial peptide (e.g., bacteriocin). The microfluidic system can comprise a transcription station configured to perform in vitro transcription, the transcription station comprising a transcription reagent. The microfluidic system can further comprise a translation station in fluid communication with the transcription station, the translation station configured to perform in vitro translation, and comprising a translation reagent. The microfluidic system can further comprise a culture station in fluid communication with the translation station and configured to culture a microbial organism in a solution environment comprising the microbial organism, a candidate nucleic acid encoding a candidate antimicrobial peptide, and the candidate antimicrobial peptide under selected culture conditions. The microfluidic system can further comprise a detector in fluid communication with the culture station, and configured to detect inhibition of growth and/or reproduction, or a lack thereof, of the microbial organism in the solution environment. The microfluidic system can further comprise a variant station configured to produce a variant nucleic acid of the nucleic acid encoding the candidate antimicrobial peptide. The variant station can be in fluid communication with the transcription station. In some embodiments, the microfluidic system further comprises a processor. The variant station can further comprise a sequencing module configured to obtain sequence information from the candidate nucleic acid. The processor can be configured to index the sequence information to the detection of inhibition of growth and/or reproduction or the lack thereof for the microbial organism. In some embodiments, the processor is configured to select a sequence of the variant nucleic acid based on the indexed information by machine learning, such as automatic machine learning. In some embodiments, the variant station produces the variant nucleic acid only if the detector detects an inhibition of growth and/or reproduction of the microbial organism in the solution environment. In some embodiments, the variant station comprises a degenerate polymerase. In some embodiments, two or more of the transcription station, the translation station, the culture station, and/or the variant station are comprised within discrete chambers that are separate from each other. In some embodiments, the transcription station and the translation station are the same station or overlap with each other. In some embodiments, two or more of the transcription station, the translation station, the culture station, and the variant station, are comprised within a single chamber. In some embodiments, the single chamber is configured to expel a first set of reagents and subsequently receive a second set of reagents that is different from the first set. In some embodiments, the selected culture conditions of the culture station comprise conditions of an industrial process, pharmaceutical manufacturing process, or mammalian microbiota. In some embodiments, the translation station is in fluid communication with a substrate comprising the candidate nucleic acid immobilized thereon. In some embodiments, the substrate comprises a bead, nanoparticle, well, membrane, nitrocellulose, PVDF, nylon, acetate derivative, matrix, pore, plastic, metal, glass, polymer, polysaccharide, or paramagnetic compound. In some embodiments, the translation station comprises a chamber that is microliter-scale and/or the solution environment is microliter-scale. In some embodiments, the solution environment has a volume of about 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl. In some embodiments, the chamber of the translation station has a volume of about 1 μl-1000 μl, 1 μl-50 μl, 1 μl-100 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl. In some embodiments, the translation station comprises a mixture of different candidate antimicrobial peptides. In some embodiments, the microbial organism comprises multiple species of microbial organisms. In some embodiments, the translation station comprises one or more post-translational modification enzymes. In some embodiments, the solution environment comprises a microdrop. In some embodiments, the microfluidic system is configured to move and combine microdrops within the system. In some embodiments, the transcription reagent and/or the translation reagent are each in separate microdrops or in a combined microdrop. In some embodiments, the culture station is configured to culture a microbial organism in a microdrop. In some embodiments, a microdrop comprises a nanometer-scale droplet of solution. In some embodiments, for any microfluidic system described herein, the antimicrobial peptide comprises, consists essentially of, or consists of a bacteriocin. In some embodiments, the microfluidic system does not comprise any cells comprising nucleic acid encoding the antimicrobial peptide.

Some embodiments include a kit for engineering an antimicrobial peptide (e.g., bacteriocin). The kit can comprise a candidate nucleic acid encoding a candidate bacteriocin. The kit can further comprise a microfluidic system as described herein. In some embodiments, the kit further comprises a library of candidate nucleic acids. In some embodiments, the kit further comprises a microbial organism. In some embodiments, the microbial organism comprises a bacteria. In some embodiments, for any kit described herein, the antimicrobial peptide comprises, consists essentially of, or consists of a bacteriocin.

### BRIEF DESCRIPTION OF THE DRAWINGS

FIG. 1 is a flow diagram illustrating a method of engineering antimicrobial peptides (e.g., bacteriocins) according to some embodiments herein.

FIG. 2 is a schematic diagram of a system for engineering antimicrobial peptides (e.g., bacteriocins) according to some embodiments herein.

### DETAILED DESCRIPTION

Described herein are methods, systems and kits for engineering antimicrobial peptides (e.g., bacteriocins). The antimicrobial peptides may be engineered to have a particular activity for a specified culture, environmental condition(s), and/or a range of conditions. For example, antimicrobial peptides can be engineered to have antimicrobial activity against pathogenic bacteria under conditions of a human gut microbiota. In some embodiments, an antimicrobial peptide is transcribed and translated in vitro and tested for effects on the viability or growth of a microbial organism under a specified set of conditions in a solution environment (for example, a droplet or microdrop in a microfluidic system). Based on the results of the testing, variants of the antimicrobial peptide can be designed, and the variants can be tested in an iterative process until a desired activity is achieved. In some embodiments, the iterative process for engineering antimicrobial peptides is performed by automated machine learning.

### Translation Solutions and Translation Stations

Translation solutions can be useful for translating nucleic acids in accordance with the methods, microfluidic systems and kits of some embodiments described herein. Suitable translation solutions can comprise, consist essentially of, or consist of reagents for in vitro translation (which, for convenience, may be referred to herein as “translation reagents”), and as such can be configured for in vitro translation of a transcript such as an RNA. In some embodiments, a translation solution is comprised by a translation station of a microfluidic system as describe herein. In some embodiments, the translation solution further comprises a transcription solution comprising reagents for transcription (which, for convenience, may be referred to herein as “transcription reagents”), and thus is configured for in vitro transcription and translation, for example to transcribe and translate a candidate nucleic acid encoding a candidate antimicrobial peptide as described herein. It is contemplated that in vitro transcription and translation in a single solution (such as a transcription solution further comprising a translation solution as described herein) can facilitate efficient in vitro production of candidate antimicrobial peptides in accordance with methods, systems, and kits of some embodiments.

In accordance with the methods, systems and kits of some embodiments described herein, the translation solution comprises, consists essentially of, or consists of one or more translation reagents Examples of translation reagents include a ribosome, a buffer, an amino acid, a tRNA (which may be conjugated to an amino acid), a lysate or extract such as an E. coli lysate or E. coli extract, and a cofactor or metallic ion such as Mg²⁺ or a combination of two or more of any of the listed items. In accordance with the methods, systems and kits of some embodiments described herein the translation solution further comprises a transcription solution, and thus is configured for in vitro transcription and translation. As described herein, a transcription solution further comprising a translation solution contemplates a single solution that is suitable for in vitro transcription and translation. As such, a transcription solution further comprising a translation solution encompasses a single transcription/translation solution, and well as translation solution with discrete sub-environments, at least some of which are suitable for transcription. It will be appreciated that some components of a transcription and/or translation solution, for example ribosomes, may not be liquids, and could potentially be isolated from the transcription and/or translation solution, for example by filtration and/or centrifugation. Translation solutions of methods, systems and kits of some embodiments described herein (and which can be comprised by translation solutions as described herein) can comprise, consist essentially or, or consist of one or more transcription reagents. Examples of transcription reagents include an RNA polymerase, a buffer, a nucleic acid mix (for example, NTPs including ATP, GTP, CTP, and UTP), a cofactor or metallic ion such as Mg²⁺ a transcription inducer (such as a transcription factor, IPTG, or lactose), a polyadenylation enzyme, a capping enzyme, a lysate or extract such as a bacterial lysate or extract such as an E. coli lysate or E. coli extract, an SP6 polymerase, a T3 polymerase, a T7 RNA polymerase, or a mixture of two or more of any of the listed items. The transcription solution can be useful for transcribing a template, such as a candidate nucleic acid as described herein. Translation solutions of methods, kits, and systems of some embodiments include one or more transcription reagents in combination with one or more translation reagents.

In some embodiments, the translation solution comprises a post-translational modification enzyme. Examples of post-translational modification enzymes include, but are not limited to a cleavage enzyme, a kinase, a phosphatase, a glycosyltransferase, or a mixture of any two of the listed items.

In some embodiments, a translation station of a microfluidic device comprises the translation solution. In some embodiments, the microfluidic device comprises a transcription station and a transcription solution, which may be the same station, or may be different stations. For example, in some embodiments, the transcription station comprises a single transcription/translation station configured for in vitro transcription and translation of a nucleic acid. In some embodiments, the translation station is configured to perform in vitro translation. In some embodiments, the transcription station is configured to perform in vitro transcription. In some embodiments, the translation station further comprises the transcription station (for example as a single environment, or as two discrete environments), and is configured to perform in vitro transcription and translation. In some embodiments, the microfluidic device comprises a transcription station comprising the transcription solution, and a separate translation station comprising the translation solution.

In some embodiments, the translation station is configured to receive a translation solution and/or one or more translation reagents and/or transcription reagents as described herein. In some embodiments, the transcription station is in fluid communication with one or more reservoirs comprising transcription reagents and/or translation reagents. As such, in some embodiments, a translation station initially does not include a translation solution, but is configured to receive a translation solution, or one or more reagents.

In accordance with the methods, systems and kits of some embodiments described herein, the translation solution comprises more than one candidate nucleic acid encoding a candidate antimicrobial peptide, for example encoding two or more different antimicrobial peptides that are being co-engineered together in accordance with some embodiments herein (so that a cocktail of bacteriocins can be engineered). In some embodiments, the translation solution comprises a candidate nucleic acid encoding no more than one candidate antimicrobial peptide.

It is contemplated that immobilizing a candidate nucleic acid on a substrate such as a bead can be useful for manipulation and analysis of a candidate nucleic acid and its corresponding candidate antimicrobial peptide in a microfluidic environment, for example in a microdroplet. In accordance with the methods, systems and kits of some embodiments described herein, the translation solution comprises a substrate. Examples of suitable substrates include a bead, a nanoparticle, a well, a membrane, nitrocellulose, PVDF, nylon, an acetate derivative, a matrix, a pore, plastic, metal, glass, a polymer, a polysaccharide, and a paramagnetic compound, or a combination of two or more of any of the listed items. In some embodiments, the candidate nucleic acid is immobilized on the substrate.

In accordance with the methods, systems and kits of some embodiments described herein, the translation solution is at a microliter-scale. For example, the translation solution may have a volume of 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl.

### Bacteriocins and Antimicrobial Peptides

As used herein, “bacteriocin,” and variations of this root term, has its customary and ordinary meaning as understood by one of skill in the art in view of this disclosure. It refers to a polypeptide that is secreted by a host cell and can neutralize at least one microbial organism other than the individual host cell in which the polypeptide is made, including cells clonally related to the host cell and other microbial cells. “Bacteriocin” also encompasses a cell-free or chemically synthesized version of such a polypeptide, for example an engineered bacteriocin in accordance with some embodiments herein. A host cell can exert cytotoxic or growth-inhibiting effects on one or a plurality of other microbial organisms by secreting bacteriocins. Example bacteriocins are set forth in SEQ ID NOS: 4-450 (even numbers) and 699-737 (odd numbers). Example nucleic acids encoding these bacteriocins are provided as SEQ ID NOs: 5-451 (odd numbers) and 700-738 (even numbers). Detailed descriptions of bacteriocins and some polynucleotide sequences that encode bacteriocins, including methods and compositions for using bacteriocins to control the growth of microbial cells can be found, for example, in U.S. Pat. No. 9,333,227, which is hereby incorporated by reference in its entirety. Some examples of suitable bacteriocins and categories of bacteriocins are taught in Tables 1.1 and 1.2 of U.S. Pat. No. 9,333,227. It is contemplated that any of these bacteriocins can be subject to further engineering. For example, variants and/or modifications of these bacteriocins can be candidate bacteriocins that can be used to engineer a bacteriocin having at least a specified activity in a solution environment under selected culture conditions in accordance with some embodiments herein. As used herein a “candidate bacteriocin” refers to a polypeptide that may have, but has not necessarily been confirmed to have bacteriocin activity. A candidate bacteriocin can be confirmed to inhibit growth and/or reproduction of a microbial organism under selected culture conditions in accordance with methods, systems, and kits of some embodiments herein. Accordingly, it is contemplated that that for any bacteriocin described herein, a “candidate bacteriocin” can comprise the noted structures, and/or can be a candidate for having the noted functionality (the functionality can subsequently be confirmed). A “variant antimicrobial peptide” or “variant bacteriocin” refers to an antimicrobial peptide (or bacteriocin, in particular) that has a different sequence and/or post-translational modification than a reference candidate bacteriocin. A “variant nucleic acid” refers to a nucleic acid that encodes the variant antimicrobial peptide (or bacteriocin, in particular). It will be appreciated that a variant of a candidate antimicrobial peptide (or candidate bacteriocin, in particular) may be selected as described herein, and as such, a variant nucleic acid of some embodiments is selected in order to encode the variant of the candidate antimicrobial peptide (or bacteriocin). If a variant antimicrobial peptide (e.g., variant bacteriocin) is tested, used, or cultured in accordance with methods, kits, and systems of some embodiments, the variant antimicrobial peptide itself can be a candidate antimicrobial peptide. For example, the variant antimicrobial peptide (e.g., variant bacteriocin) can comprise a point mutation, deletion (including a truncation), insertion (including a C- or N-terminal addition), rearrangement, or two or more of these compared to a candidate antimicrobial peptide.

Antimicrobial peptides are a class of peptides that kill or arrest the growth of microbial organisms. As used herein “antimicrobial peptide” (including variations of this root term) has its customary and ordinary meaning as would be understood by one of ordinary skill in the art in view of this disclosure. Clasically, antimicrobial peptides have been described as peptides produced by the innate immune systems of invertebrates and vertebrates. Thus, while bactericins have classically been referred to a class of microbial gene products that target microbial organisms, antimicrobial peptides have classically been referred to as a class of invertebrate and vertebrate gene products that target microbial organisms. However, for conciseness “antimicrobial peptide” as used herein broadly encompasses classical antimicrobial peptides (e.g., that confer innate immune activity against microbial organisms) as well as bacteriocins.

Examples of classical antimicrobial peptides suitable for methods, systems, and kits of some embodiments herein are known in the art, and can be found, for example, at The Antimicrobial Peptide Database accessible on the world wide web at aps.unmc.edu/AP/, which is incorporated herein by reference in its entirety. Over 1000 antimicrobial peptides and variants thereof have been identified and cataloged. The Antimicrobial Peptide Database is described in Wang et al. (2016), Nucleic Acids Res. 44 (Database issue): D1087-D1093, which is incorporated herein by reference in its entirerty. Examples of antimicrobial peptides include bacteriocins, antibacterial, antiviral, anti-HIV, antifungal, antiparasitic and anticancer peptides, such as Dermaseptin-B2, Abaecin, Ct-AMP1, Andropin, Aurein 1.1, Lactoferricin B, and Heliomicin. Methods, compositions, systems, and microfluidic devices of some embodiments comprise naturally-occuring antimicrobial peptides, or a nucleic acid encoding the same. Methods, compositions, systems, and microfluidic devices of some embodiments comprise non-naturally occurring antimicrobial peptides, or nucleic acids encoding the same. Methods, compositions, systems, and microfluidic devices of some embodiments include antimicrobial peptides that comprise a mutation or variation in a naturally-occuring antimicrobial peptides, or a nucleic acid encoding the same. Methods, compositions, systems, and microfluidic devices of some embodiments comprise antimicrobial peptides comprising, consisting essentially of, or consisting of non-naturally occuring peptide sequences, or nucleic acids encoding the same.

In accordance with the methods, systems and kits of some embodiments described herein, an antimicrobial peptide, for example, a bacteriocin to be engineered or that has been engineered (or a candidate antimicrobial peptide, and/or a variant of a candidate antimicrobial peptide as described herein) of some embodiments is initially produced in a pro-polypeptide, which can then be cleaved to produce the antimicrobial peptide. In some embodiments, the pro-polypeptide or antimicrobial peptide is chemically synthesized. In some embodiments, an antimicrobial peptide comprises a polypeptide that has undergone post-translational modifications, for example cleavage, or the addition of one or more functional groups. In some embodiments, an antimicrobial peptide is engineered. In some embodiments, the engineered antimicrobial peptide is engineered to have a modified activity or ability to kill or affect the growth of a microbial organism.

Some antimicrobial peptides (such as bacteriocins) have cytotoxic activity (e.g. “bacteriocide” effects), and thus can kill microbial organisms, for example bacteria, yeast, algae, synthetic microorganisms, and the like. Some antimicrobial peptides (such as bacteriocins) can inhibit the reproduction of microbial organisms (e.g. “bacteriostatic” effects), for example bacteria, yeast, algae, synthetic microorganisms, and the like, for example by arresting the cell cycle.

While many the bacteriocins are naturally-occurring (for example, naturally occurring bacteriocins set forth in SEQ ID NOS: 4-450 (even numbers) and 699-737 (odd numbers)), the skilled artisan will appreciate that in some embodiments of the methods, systems and kits described herein, a bacteriocin comprises a naturally-occurring bacteriocin other than the bacteriocins and encoding nucleotide sequences of SEQ ID SEQ ID NOS: 4-450 (even numbers) and 699-737 (odd numbers), or a non-naturally-occurring bacteriocin or a synthetic bacteriocin (such as an engineered bacteriocin), or a variant thereof (which can also be a kind of engineered bacteriocin of some embodiments). In some embodiments, an engineered bacteriocin has enhanced or decreased levels of cytotoxic or growth inhibition activity on the same or a different microorganism or species of microorganism relative to a wild-type bacteriocin. In some embodiments, the antimicrobial peptide (or bacteriocin) does not comprise a lantibiotic.

Several motifs have been recognized as characteristic of bacteriocins. For example, the motif YGXGV (SEQ ID NO: 2), wherein X is any amino acid residue, is an N-terminal consensus sequence characteristic of a class IIa bacteriocin. Accordingly, in some embodiments, a candidate (or variant) bacteriocin (e.g., an engineered bacteriocin) comprises an N-terminal sequence with at least about 50% identity to SEQ ID NO: 2), for example at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 2). In some embodiments, a candidate (or variant) bacteriocin (e.g., an engineered bacteriocin) comprises a N-terminal sequence comprising SEQ ID NO: 2). Additionally, some class IIb bacteriocins comprise a GxxxG motif. Without being limited by any particular theory, it is believed that the GxxxG motif can mediate association between helical proteins in the cell membrane, for example to facilitate bacteriocin-mediated neutralization through cell membrane interactions. As such, in some embodiments, the bacteriocin (e.g., the engineered bacteriocin) comprises a motif that facilitates interactions with the cell membrane. In some embodiments, the bacteriocin comprises a GxxxG motif. Optionally, the bacteriocin comprising a GxxxG motif can comprise a helical structure. In addition to structures described herein, “bacteriocin” as used herein also encompasses structures that have substantially the same effect on microbial cells as any of the bacteriocins explicitly provided herein.

In some embodiments, a fusion polypeptide comprising two or more antimicrobial peptides (such as bacteriocins) or portions thereof has a neutralizing activity against a broader range of microbial organisms than either individual antimicrobial peptide of the two or more antimicrobial peptides or portions thereof. For example, it has been shown that a hybrid antimicrobial peptide displays antimicrobial activity against pathogenic Gram-positive and Gram-negative bacteria (Acuña et al. (2012), FEBS Open Bio, 2:12-19). It is noted that that Ent35-MccV fusion bacteriocin comprises, from N-terminus to C-terminus, an N-terminal glycine, Enterocin CRL35, a linker comprising three glycines, and a C-terminal Microcin V.

It is contemplated herein that an antimicrobial peptide (such as a bacteriocin) can comprise a fusion of two or more polypeptides, for example two or more polypeptides having antimicrobial (such as bacteriocin) activity. In some embodiments an antimicrobial peptide or a candidate antimicrobial peptide comprises a chimeric protein. In some embodiments, a variant antimicrobial peptide (such as a bacteriocin) or an engineered antimicrobial peptide (such as an engineered bacteriocin) comprises a fusion polypeptide comprising two or more antimicrobial peptides (such as bacteriocins). In some embodiments, a variant antimicrobial peptide (such as a bacteriocin) or an engineered antimicrobial peptide (such as a bacteriocin) comprises a chimeric protein comprising two or more antimicrobial peptides (such as bacteriocins), or fragments thereof. In some embodiments, the two or more antimicrobial peptides of the fusion comprise polypeptides of SEQ ID NOS: 4-450 (even numbers) and 699-737 (odd numbers), and or encoded by nucleic acids of SEQ ID NOs: 5-451 (odd numbers) and 700-738 (even numbers), or variants or modifications thereof. In some embodiments, the fusion polypeptide has a broader spectrum of activity than either individual antimicrobial peptide, for example having neutralizing activity against more microbial organisms, neutralizing activity under a broader range of environmental conditions, and/or a higher efficiency of neutralization activity. In some embodiments, the fusion polypeptide comprises two, three, four, five, six, seven, eight, nine, or ten antimicrobial peptides. In some embodiments, two or more antimicrobial peptide polypeptides are fused to each other via a covalent bond, for example a peptide linkage. In some embodiments, a linker is positioned between the two individual antimicrobial polypeptides of the fusion polypeptide. In some embodiments, the linker comprises one or glycines, for example about 1, 2, 3, 4, 5, 6, 7, 8, 9, 10, 11, 12, 13, 14, 15, 16, 17, 18, 19, or 20 glycines. In some embodiments, the linker is cleaved within the cell to produce the individual antimicrobial peptides (such as bacteriocins) included in the fusion protein. In some embodiments, a variant antimicrobial peptide (such as a variant bacteriocin) or engineered antimicrobial peptide (such as an engineered bacteriocin) as provided herein comprises a modification to provide a desired spectrum of activity relative to the unmodified or candidate antimicrobial peptide (e.g., bacteriocin). For example, the variant antimicrobial peptide (e.g., bacteriocin) or engineered antimicrobial peptide (e.g., bacteriocin) may have enhanced or decreased activity against the same organisms as the unmodified or candidate antimicrobial peptide (e.g., bacteriocin). Alternatively, the modified antimicrobial peptide (e.g., bacteriocin) may have enhanced activity against an organism against which the unmodified or candidate antimicrobial peptide (e.g., bacteriocin) has less activity or no activity.

In accordance with the methods, systems and kits of some embodiments described herein, an antimicrobial peptide (e.g., bacteriocin) is encoded by a polynucleotide. For example, a DNA sequence of an antimicrobial peptide (e.g., bacteriocin) gene may encode an mRNA transcript that is translated into a protein comprising, consisting essentially of, or consisting of an antimicrobial peptide (such as a bacteriocin). As used herein, a “candidate nucleic acid” refers to a nucleic acid that encodes a “candidate antimicrobial peptide (e.g., bacteriocin)” as described herein. In some embodiments, the polynucleotide encoding an antimicrobial peptide (such as a bacteriocin) comprises a variant nucleic acid. In some embodiments, the variant nucleic acid is produced by engineering or modifying the sequence of the candidate nucleic acid, for example by synthesis of the variant nucleic acid sequence, or by mutagenesis such a chemical mutagenesis, or degenerate polymerization.

### Microbial Organisms

Engineered antimicrobial peptides in accordance with methods, microfluidic systems, and kits of some embodiments herein inhibit the growth and/or reproduction of microbial organisms. As used herein, “microbial organism,” “microorganism,” and variations of these root terms (such as pluralizations and the like), any naturally-occurring species or fully synthetic prokaryotic or eukaryotic unicellular organism, as well as Archaea species, as well as genetic modifications of any of these. Thus, this expression can refer to cells of bacterial species, fungal species, and algae.

Exemplary microorganisms that can be used in accordance with methods, kits, and microfluidic systems of some embodiments herein include, but are not limited to, bacteria, fungus, and algae, for example photosynthetic microalgae. In some embodiments, a microorganism is selected from the group consisting of bacteria and fungus (such as yeast). Furthermore, fully synthetic microorganism genomes can be synthesized and transplanted into single microbial cells, to produce synthetic microorganisms capable of continuous self-replication (see Gibson et al. (2010), “Creation of a Bacterial Cell Controlled by a Chemically Synthesized Genome,” Science 329:52-56, hereby incorporated by reference in its entirety). As such, in some embodiments, the microorganism is fully synthetic. A desired combination of genetic elements, including elements that regulate gene expression, and elements encoding gene products (for example antimicrobial peptides (e.g., bacteriocins), immunity modulators, poison, antidote, and industrially useful molecules) can be assembled on a desired chassis into a partially or fully synthetic microorganism. Description of genetically engineered microbial organisms for industrial applications can also be found in Wright, et al. (2013) “Building-in biosafety for synthetic biology” Microbiology 159:1221-1235.In the methods, microfluidic systems and kits of some embodiments, two or more different species and/or strains of microbial organism are contemplated, for example a solution environment can comprise two or more different species of microbial organisms, such as different members of a microbiota as described herein and/or two or more different species or strains that participate in an industrial fermentation process. For example, some embodiments include two or more different species and/or strains of bacteria, yeast, and/or algae.

A variety of bacterial species and strains can be used in accordance with methods, microfluidic systems, and kits of some embodiments herein, along with genetically modified variants, or synthetic bacteria based on a “chassis” of a known species. Exemplary bacteria with industrially applicable characteristics, which can be used in accordance with methods, systems, and kits of some embodiments herein include, but are not limited to, Bacillus species (for example Bacillus coagulans, Bacillus subtilis, and Bacillus licheniformis), Paenibacillus species, Streptomyces species, Micrococcus species, Corynebacterium species, Acetobacter species, Cyanobacteria species, Salmonella species, Rhodococcus species, Pseudomonas species, Lactobacillus species, Enterococcus species, Alcaligenes species, Klebsiella species, Paenibacillus species, Arthrobacter species, Corynebacterium species, Brevibacterium species, Thermus aquaticus, Pseudomonas stutzeri, Clostridium thermocellus, Staphylococcus species such as Staphylococcus aureus, and Escherichia coli. In some embodiments, the bacterial species are species of a microbiota as described herein, for example the microbiota of a mammalian organ such as a gastrointestinal or skin micobiota. For example, in some embodiments, the bacterial species of a gastrointestinal microbiota include Firmicutes and/or Bacteriodetes.

A variety of yeast species and strains can be used in accordance with methods, microfluidic systems, and kits of some embodiments herein, and genetically modified variants, or synthetic yeast based on a “chassis” of a known species are also contemplated. Exemplary yeast with industrially applicable characteristics, which can be used in accordance with embodiments herein include, but are not limited to Saccharomyces species (for example, Saccharomyces cerevisiae, Saccharomyces bayanus, Saccharomyces boulardii), Candida species (for example, Candida utilis, Candida krusei), Schizosaccharomyces species (for example Schizosaccharomyces pombe, Schizosaccharomyces japonicas), Pichia or Hansenula species (for example, Pichia pastoris or Hansemdla polymorpha) species, and Brettanomyces species (for example, Brettanomyces claussenii).

A variety of algae species and strains can be used in accordance with methods, microfluidic systems, and kits of some embodiments herein, and genetically modified variants, or synthetic algae based on a “chassis” of a known species are also contemplated. In some embodiments, the algae comprises photosynthetic microalgae. Exemplary algae species that can be useful for biofuels, and can be used in accordance with embodiments herein, include Botryococcus braunii, Chlorella species, Dunaliella tertiolecta, Gracilaria species, Pleurochrysis carterae, and Sargassum species. Additionally, many algaes can be useful for food products, fertilizer products, waste neutralization, environmental remediation, and carbohydrate manufacturing (for example, biofuels).

In clinical and industrial settings, undesired bacteria have developed resistance to one or more antibiotics, which can make it challenging to use conventional antibiotics to control the growth of these bacteria. Without being limited by theory, it is contemplated that mechanisms that confer antibiotic resistance (for example pumps or channels that transport drug compounds) are inapplicable to antimicrobial peptides such as bacteriocins, and thus, engineered antimicrobial peptides (e.g., engineered bacteriocins) in accordance with methods, systems, and kits of some embodiments herein can inhibit the growth or reproduction of a microbial organism that is resistant to antibiotics. In some embodiments, the microbial organism is resistant to an antimicrobial compound. For example, some embodiments include bacteria resistant to an antibiotic. In some embodiments, the microbial organism is Methicillin-resistant Staphylococcus aureus (MRSA). In some embodiments, the microbial organism is Vancomycin resistant.

### Solution Environments

Solution environments can be useful for engineering antimicrobial peptides (such as bacteriocins) in accordance with the methods, microfluidic systems and kits of some embodiments described herein. For example, culture conditions in a solution environment can replicate or mimic conditions in a natural or synthetic environment in which a microbial organism may grow (for example a microbiota, an industrial process, and/or a sterile manufacturing process), and to engineer an antimicrobial peptide with selected characteristics in the natural or synthetic environment. Examples of culture conditions can include, but are not limited to temperature, pH, chemicals, the presence of other microbial organisms, and/or the presence of gene products such as proteases. Suitable solution environments can comprise, consist essentially of, or consist of one or more solutions and other ingredients that replicate or mimic the natural or synthetic environment. In some embodiments, a solution environment is comprised by a culture station of a microfluidic system as describe herein. In some embodiments, the solution environment comprises a microbial organism and a candidate antimicrobial peptide (e.g., candidate bacteriocin). The microbial organism can be cultured in the solution environment with the candidate antimicrobial peptide under selected culture conditions to replicate (or mimic) a natural or synthetic environment. In some embodiments, the solution environment is provided as a microdrop that is combined with a microdrop comprising the candidate antimicrobial peptide, so as to form a microdrop solution environment comprising the candidate antimicrobial peptide and microbial organism(s) in the solution environment.

In accordance with the methods, systems and kits of some embodiments described herein, the solution of the solution environment comprises an industrial feedstock, a pharmaceutical or cosmetic manufacturing solution, or a bodily fluid (for example, that of a microbiota). In accordance with the methods, systems and kits of some embodiments described herein, the solution of the solution environment comprises, consists essentially of, or consists of water, oil, acetic acid, methanol, ethanol, propanol, butanol, formic acid, propylene carbonate, nitromethane, dimethyl sulfoxide, acetonitrile, dimethylformamide, acetone, ethyl acetate, tetrahydrofuran, dichloromethane, diethyl ether, chloroform, 1,4-dioxane, toluene, benzene, cyclohexane, hexane, cyclopentane, pentane, a feedstock, a culture medium, a bodily fluid, or a combination of two or more of any of the listed items.

In accordance with the methods, systems and kits of some embodiments described herein, the selected culture conditions of the solution environment comprise, consists essentially of, or consist of conditions of an industrial process, a pharmaceutical manufacturing process, or a microbiota. Accordingly, the engineered antimicrobial peptide(s), when produced, can be integrated into the corresponding industrial process (which can be on an industrial scale).

Examples of industrial processes whose conditions may be replicated in a solution environment of some embodiments include fermentation (for example carbohydrate manufacturing such as biofuels), food production, decomposition, waste neutralization, and environmental remediation.

Examples of pharmaceutical or biologic or cosmetic manufacturing processes whose conditions may be replicated in solution environments of some embodiments include chemical synthesis, tissue culture, fermentation, extraction, isolation of chemical compounds, proportioning, and packaging, or a combination of two or more of any of the listed items. In some embodiments, a solution environment replicates a pharmaceutical or biologic product or component thereof, for example a buffer, a sterile solution, a saline solution, an ointment, a capsule, a container, and a vial, or a combination of two or more of any of the listed items. It can be advantageous for engineered antimicrobial peptides of some embodiments to maintain a sterile pharmaceutical or biologic or cosmetic manufacturing processes by targeting contaminating microbes.

The composition of microbial organisms in a solution environment of methods, microfluidic systems, and kits of some embodiments can be controlled so that a solution environment contains a specified species, quantities, and/or ratios of bacteria. By way of example, live bacteria can be sorted by flow cytometry, for example based on parameters such as membrane integrity (which can be measured, e.g, through labeling with a membrane-impermeable probe such as PI), membrane potential proton motive force (which can be measured, e.g., through labeling with membrane-permeable anionic or cationic dyes), enzyme activity (which can be measured, e.g., through labeling with of fluorogenic substrates that become fluorescent through enzyme activity), substrate turnover (which can be measured, e.g., through labeling with fluorescent substrates or analogs thereof), pump activity (which can be measured, e.g., through destaining in the presence of probes), gene expression activity (such as response to signaling, which can be measured, for example, by promoter-drive fluorescent protein synthesis), cell division (which can be measured, e.g., by counting fixed volumes against references particles), and/or cell proliferation (which can be measured, e.g., through DNA-specific staining). Example approaches for sorting live microbial cells are reviewed in detail in Müller et al. (2010), FEMS Microbiol. Rev. 34:554-587, which is incorporated by reference in its entirety herein. In methods, microfluidic systems, and kits of some embodiments, the composition of microbial organisms in a solution environment is adjusted by flow cytometric sorting based on membrane integrity, membrane potential proton motive force, enzyme activity, substrate turnover, pump activity, gene expression activity, cell division, cell proliferation, or any combination of two or more of the listed items.

In methods, microfluidic systems, and kits of some embodiments, the cultures conditions of the solution environment replicate those of a microbiota, such as a plant microbiota or an animal microbiota such as a mammalian microbiota (e.g., a human microbiota). Examples of a mammalian microbiota include, but are not limited to, microbiota that of a gastrointestinal tract, skin, a mammary gland, a placenta, a tissue, a biofluid, a seminal fluid, a uterus, a vagina, an ovarian follicle, a lung, saliva, an oral cavity, a mucosa, a conjunctiva, and a biliary tract, or a combination of two or more of any of the listed items.

In some embodiments, the solution environment comprises, consists essentially of, or consists of a microbial organism. In some embodiments, the solution environment comprises at least two species or strains of microbial organism.

In some embodiments, the solution environment is at a microliter-scale. For example, the solution environment may have a volume of 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl. In some embodiments, the solution environment comprises substances such as trace metals, ions, and/or metabolites. In some embodiments, the solution environment comprises no such substances. In some embodiments, the solution environment comprises a media such as LB media or DMEM in a liquid form or in a gel or matrix form. In some embodiments, the solution environment is provided as a microdrop that is combined with a microdrop comprising the candidate antimicrobial peptide (e.g., candidate bacteriocin), so as to form a microdrop solution environment comprising the candidate antimicrobial peptide (e.g., candidate bacteriocin) and microbial organism(s) in the solution environment. Optionally, the solution environment can be provided as a single microdrop. Optionally, the solution environment can be provided as two or more mcirodrops (which contain the same or different components). For example, in some embodiments, two or more microdrops containing different components (e.g., a microbial organism, a culture medium, an industrial product or intermediate, or the like) are combined microdrop, and then the combined microdrop is contacted with a microdrop comprising the candidate antimicrobial peptide (e.g., candidate bacteriocin). For example, in some embodiments, two or more different microdrops (comprising the same or different components) can be added to a microdrop comprising the antimicrobial peptide, so as to form the solution environment comprising the microbial organism. Without being limited by theory, it is contemplated that some microbial organisms can inhibit in vitro transcription and/or in vitro translation. Accordingly, in accordance with methods, systems, and kits of some embodiments herein, an antimicrobial peptide (such as a bacteriocin) is produced by in vitro transcription before it is put in a solution environment with a microbial organism. For example, an antimicrobial peptide (such as a bacteriocin) can be transcribed/translated in a microdrop in vitro, and then the antimicrobial peptidecan be contacted or mixed with a microdrop comprising the microbial organism.

Examples of culture conditions that can be selected in the solution environment of methods, microfluidic devices, and kits some embodiments include, but are not limited to temperature, pH, viscosity, osmolality, salinity, humidity, culture time, wind, air flow, and turbidity, or a combination of two or more of any of the listed items. It is noted that the culture conditions can be selected for a particular setpoint (e.g., hold at or about 37° C.), threshold (e.g., no higher than 37° C.), or range (e.g., 0-37° C.). Examples of selected culture conditions of a solution environment include room temperature, body temperature, standard temperature and pressure, acidic conditions, alkaline conditions, pH (e.g., neutral pH), viscosity, salinity, humidity, darkness, turbidity, or a combination of two or more of any of the listed items. In some embodiments, the culture conditions comprise the presence of one or more other microbial organisms, which may be of the same species as each other, the same species (but different strains) from each other, different species from each other, and/or a combination of any of these. In some embodiments, the culture conditions comprise the presence of a gene product, for example a protease. It is contemplated that proteases or other gene products in solution environments can inhibit or prevent antimicrobial peptides (such as bacteriocins) from functioning, and accordingly, it can be useful in some embodiments to engineer antimicrobial peptides that are resistant to protease(s) in an a solution environment that comprises one or more proteases. Similarly, it is contemplated that the solution environment may comprise other gene products that may affect antimicrobial peptide function, for example post-translational modification enzymes, and in some embodiments, it can be useful to engineer antimicrobial peptides that function in the presence of these gene products. Accordingly, in some embodiments, the solution environment contains one or more gene products (for example a protease and/or a post-translational modification enzyme), and antimicrobial peptides are engineered to inhibit the growth or reproduction of microbial organisms in the presence of the gene product.

Detecting Inhibition of Growth and/or Reproduction, or a Lack Thereof

### Inhibition of Growth and or Reproduction

In some embodiments of the methods, systems and kits described herein, a candidate antimicrobial peptide (such as a bacteriocin) and/or an engineered antimicrobial peptide (such as a bacteriocin) inhibits the growth and/or reproduction of a microbial organism (for example a microbial organism as described herein), in a solution environment under selected culture conditions. Inhibition of growth or reproduction has its customary and ordinary meaning as understood by one of skill in the art in view of this disclosure. It refers to a decrease in or arrest of proliferation of microbial organisms (or a decrease in the rate of proliferation of microbial organisms), for example, arrest of the cell cycle and/or killing of microbial organisms. In accordance with methods, systems, and kits of some embodiments herein, an inhibition of growth and/or reproduction of a microbial organism represents a quantity of microbial organisms, and/or a rate of growth of the microbial organisms that does not exceed a reference level. In some embodiments of the methods, systems and kits described herein, inhibition of growth comprises a quantity of a microbial organism remaining constant or decreasing over time. The decrease can be compared to a reference level from an earlier point in time.

In some embodiments, inhibition of growth comprises a decrease in the size or amount of the microbial organism. In some embodiments, inhibition of growth comprises a decrease in an organelle of the microbial organism, for example a chloroplast or mitochondrion. In some embodiments, inhibition of growth comprises killing the microbial organism, for example through lysis, apoptosis, and/or necrosis. In some embodiments, inhibition of reproduction of a microbial organism comprises a decrease or a cessation in the rate of cell division or cell doubling. In some embodiments, inhibition of reproduction of a microbial organism comprises a decrease or a cessation in an increase in an amount of the microbial organism.

### Detecting Inhibition of Growth and or Reproduction

Inhibition of growth and/or reproduction, or a lack thereof, of a microbial organism can be detected directly or indirectly via a number of suitable approaches and apparatuses in accordance with methods, microfluidic systems, and kits of some embodiments herein. By way of example, inhibition of growth or reproduction of one or more microbial organisms can indicate whether a candidate antimicrobial peptide (such as a bacteriocin) has a suitable activity in a particular solution environment under selected culture conditions in accordance with the methods, microfluidic systems and kits of some embodiments described herein. Detecting inhibition of growth and/or reproduction, or a lack thereof, can be performed by any number of suitable methods, for example as described herein.

In methods, kits, and microfluidic systems of some embodiments, inhibition of growth and/or reproduction is detected when a quantity, growth rate, or reproduction rate of a microbial organism is less than, or is less than or equal to a predetermined level. The predetermined level can be a reference point. For example, the predetermined level of some embodiments can be a growth rate or quantity of the microbial organism prior to culturing the microbial organism with the antimicrobial peptide (such as a bacteriocin). For example, the predetermined level of some embodiments can be or the growth rate or quantity of a control microbial organism that is cultured in a control solution environment under the selected culture conditions in the absence of the candidate antimicrobial peptide (and/or in the presence of a sham antimicrobial peptide such as a sham bacteriocin that is known to be inactive). In some embodiments, the predetermined level of inhibition of growth and/or reproduction of the microbial organism in the solution environment under the selected culture conditions is a greater level of inhibition than that of a reference naturally-occurring or engineered antimicrobial peptide (such as an engineered bacteriocin) in a corresponding control solution environment containing the same microbial organism under the same culture conditions.

In accordance with the methods, systems and kits of some embodiments described herein detecting inhibition of growth and/or reproduction, or a lack thereof, of the microbial organism comprises quantifying the microbial organism in the solution environment. A decrease (or arrest) in a quantity of the microbial organism in the solution environment over a period of time can indicate inhibition of growth and/or reproduction of the microbial organism. Quantifying the microbial organism may be performed by any method known in the art. In some embodiments of the methods, systems and kits described herein, quantifying the microbial organism comprises detecting and/or measuring the light absorbance of a bacterial culture. In some embodiments, the quantity of the microbial organism is detected by measuring an optical density with a spectrophotometer (for example at OD₆₀₀). In some embodiments, quantifying the microbial organism comprises determining the amount of a microbial marker such as a protein, RNA sequence or DNA sequence. For example, in some embodiments, quantifying the microbial organism comprises performing RNA or DNA sequencing or qPCR. In some embodiments, quantifying the microbial organism comprises optically, chemically, and/or electromagnetically quantifying the marker (for example, by performing an immunoassay, by performing an enzymatic assay, via chromatography, via mass spectrometry, or the like). In some embodiments, quantifying the microbial organism comprises visually detecting the microbial organism. In some embodiments, a detector such as an optical sensor detects inhibition of growth and/or reproduction, or a lack thereof, of a microbial organism as described herein.

### Sequencing Candidate Nucleic Acids, Indexing, and Variant Antimicrobial Peptides

In accordance with the methods, systems and kits of some embodiments described herein, candidate nucleic acids are sequenced. Sequencing candidate nucleic acids can facilitate the selection of variant antimicrobial peptides (such as bacteriocins) and variant nucleic acids as described herein. Sequencing can be performed by any method known in the art, for example chain termination sequencing (which may also be referred to as “Sanger sequencing”), and/or next generation sequencing using a platform such as Roche 454, Illumina Solexa, ABI-SOLID, Ion Torrent, Complete Genomics, Pacific Bioscience, Helicos, and/or the Polonator platform. In methods, microfluidic systems, and kits of some embodiments, sequencing comprises chain termination sequencing, pyrosequencing, sequencing by synthesis, sequencing by ligation, nanopore sequencing, ion semiconductor sequencing or single-molecule real-time sequencing.

It is further contemplated that as successive rounds of culturing and detection are performed on candidate antimicrobial peptides (such as candidate bacteriocins) and/or variant antimicrobial peptides (such as variant bacteriocins), structural features of the candidate antimicrobial peptides and/or variant antimicrobial peptides that positively (or negatively) correlate with inhibition of growth of a microbial organism in a solution environment under selected culture conditions can guide the design of variant antimicrobial peptides such as variant bacteriocins (and thus variant nucleic acids) having desired activity. By way of example, data can be collected and indexed through successive iterations of engineering candidate nucleic acids, and automated machine learning can identify structures such as domains and structural motifs that correlate with desired inhibition of growth or reproduction (and can be included). For example, as discussed herein, certain domains and motifs have shown to be correlates of certain antimicrobial peptide (e.g., bacteriocin) activities, for example the motif YGXGV motif associated with the N-termini of class IIa bacteriocins. By way of example, domains and structural motifs that negatively correlate with desired inhibition of growth or reproduction can be avoided.

Accordingly, methods, systems and kits of some embodiments described herein comprise indexing or correlating the sequence of a candidate nucleic acid with inhibition of growth and/or reproduction, or a lack thereof, of a microbial organism. As such, structure-function correlations can be identified, which can guide the selection of variant antimicrobial peptides (such as variant bacteriocins). By way of example, particular classes of amino acids (e.g., hydrophobic, hydrophilic) and particular positions or ranges of positions, or known or canonical structural domains or motifs can be correlated with inhibition of growth or reproduction. In some embodiments, a candidate antimicrobial peptide (e.g., candidate bacteriocin) that yields inhibition of growth or reproduction that meets or exceeds a particular threshold can be aligned (for example using a ClustalW tool), and consensus sequences can be identified. As such, in accordance with methods and microfluidic systems and kits of some embodiments, a sequence of a selected candidate nucleic acid (encoding a candidate antimicrobial peptide that yields detection of inhibition of growth or reproduction of the microbial organism in the solution environment under the selected culture conditions) can be obtained. The sequence can be indexed to the detected inhibition of growth and/or reproduction of the microbial organism in a solution environment. Based on the sequence, one or more variant antimicrobial peptides (such as variant bacteriocins) can be identified (as described herein), and a variant nucleic acid encoding the variant antimicrobial peptide (such as a variant bacteriocin) as described herein can be produced.

In accordance with the methods, systems and kits of some embodiments described herein, designing a variant antimicrobial peptide (such as a variant bacteriocin) (and therefore producing a variant nucleic acid sequence) based on the indexed sequence information comprises machine learning, such as automated machine learning.

Variant nucleic acids in accordance with methods, systems, and kits of some embodiments herein can be performed by any suitable method known in the art, for example chemical synthesis, or mutagenesis of a candidate nucleic acid. In some embodiments, the variant nucleic acid is immobilized on a substrate such as a bead as described herein.

In methods, systems and kits of some embodiments, producing a variant nucleic acid comprises one or more of amplifying a selected candidate nucleic acid with a degenerate polymerase; amplifying the selected candidate nucleic acid in the presence of a degenerate primer; amplifying the selected candidate nucleic acid in the presence of a degenerate nucleotide; and a mutagenesis reaction on the selected candidate nucleic acid.

In some embodiments, producing the variant nucleic acid comprises chemical synthesis of the variant nucleic acid. In some embodiments, the chemical synthesis comprises synthesis with random nucleotides. In some embodiments, the chemical synthesis produces a specified sequence, for example a variant nucleic acid designed based on indexing the sequence of a candidate nucleic acid to inhibition of growth and/or reproduction as described herein. The specified variant nucleic acid sequence of some embodiments is designed by automated machine learning. In some embodiments, the variant nucleic acid is codon-optimized. In some embodiments, producing the variant nucleic acid comprises degenerate or mutagenesis replication or amplification of a candidate nucleic acid. In some embodiments, producing the variant nucleic acid comprises mutating a candidate nucleic acid, for example using ionizing radiation, a chemical mutagen such as an intercalating agent, or via incorporation of a synthetic nucleotide. In some embodiments, the mutagen reacts with the candidate nucleic acid and results in mutations upon replication or amplification of the candidate nucleic acid. In some embodiments, the mutagen interacts with a polymerase or a reagent involved in the replication or amplification of the candidate nucleic acid, and thereby causes the polymerase to insert incorrect nucleic acids during the replication or amplification, and results in the variant nucleic acid. In some embodiments, a degenerate polymerase produces replicates of a candidate nucleic acid that include variations.

In methods, systems and kits of some embodiments, the candidate nucleic acid or the variant nucleic acid is transcribed and translated via in vitro translation. In methods, systems and kits of some embodiments, the candidate nucleic acid or the variant nucleic acid is inserted into a vector for cellular or cell-free transcription and translation. In some embodiments, the vector is cultured to produce quantities of the candidate nucleic acid or the variant nucleic acid. Some embodiments comprise harvesting and/or isolating the quantities of the candidate nucleic acid and/or variant nucleic acid produced by the vector. In some embodiments, the vector is configured to express a candidate antimicrobial peptide (such as a candidate bacteriocin) from the candidate nucleic acid or an engineered antimicrobial peptide (such as an engineered bacteriocin) from the variant nucleic acid. In some embodiments, the candidate antimicrobial peptide (e.g., candidate bacteriocin) or the engineered antimicrobial peptide (e.g., engineered bacteriocin) encoded by the vector is harvested or isolated from the vector. In some embodiments, the vector comprises bacteria such as E. coli. Thus, some embodiments comprise growing up quantities of a candidate nucleic acid or a variant nucleic acid in a vector such as bacteria, and isolating the candidate nucleic acid, the variant nucleic acid, a candidate antimicrobial peptide (e.g., candidate bacteriocin) produced by the candidate nucleic acid, or an engineered antimicrobial peptide (e.g., engineered bacteriocin) produced by the variant nucleic acid, from the vector.

### Methods of Engineering Antimicrobial Peptides

In some embodiments, a method of engineering an antimicrobial peptide is provided. For example, the method may include engineering an antimicrobial peptide to have a desired range of activity in a desired range of culture conditions. In some embodiments, one or more of the methods of engineering an antimicrobial peptide (e.g., bacteriocin) are performed on a microfluidic system as described herein, and/or using components of a kit as described herein.

In some embodiments, the method comprises translating a candidate nucleic acid to produce a candidate antimicrobial peptide (e.g., candidate bacteriocin), culturing the candidate antimicrobial peptide with a microbial organism in a solution environment, and detecting inhibition of growth and/or reproduction (or a lack thereof) of the microbial organism. Portions of, or all of the method can be repeated on successive variants of candidate antimicrobial peptides (e.g., candidate bacteriocins) until a candidate antimicrobial peptide exhibiting a desired inhibition of growth and/or reproduction is identified, which can represent an engineered antimicrobial peptide (e.g., engineered bacteriocin).

In some embodiments, a method of engineering an antimicrobial peptide (e.g., bacteriocin) is provided. The method can comprise (a) translating a candidate nucleic acid encoding a candidate antimicrobial peptide (e.g., bacteriocin) in vitro in a translation solution, whereby the translation solution comprises the candidate antimicrobial peptide (e.g., bacteriocin), (b) combining the candidate antimicrobial peptide (e.g., bacteriocin) and a microbial organism in a solution environment, (c) culturing the microbial organism and the candidate antimicrobial peptide (e.g., bacteriocin) in the solution environment under selected culture conditions, (d) detecting inhibition of growth and/or reproduction, or a lack thereof, of the microbial organism in the solution environment, (e) selecting the candidate nucleic acid upon detection of inhibition of growth and/or reproduction of the microbial organism in the solution environment under the selected culture conditions, (f) producing a variant nucleic acid of the selected candidate nucleic acid, the variant nucleic acid encoding a variant of the candidate antimicrobial peptide (e.g., bacteriocin). Steps (a)-(f) can be repeated using one or more successive variant nucleic acids as the candidate nucleic acid, until a predetermined level of inhibition of growth and/or reproduction of the microbial organism in the solution environment under the selected culture conditions is achieved, whereby the antimicrobial peptide (e.g., bacteriocin) has been engineered.

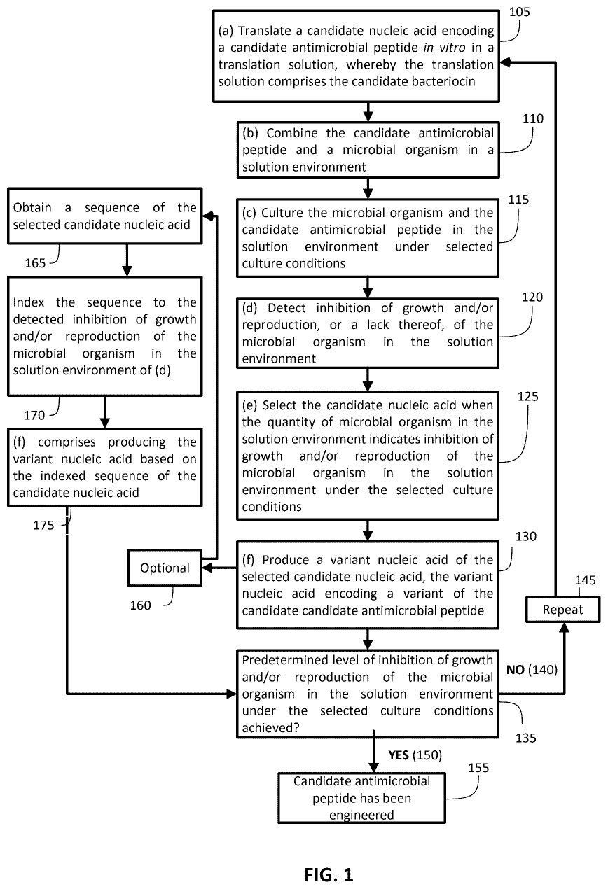

FIG. 1 is a flow diagram illustrating a method of engineering an antimicrobial peptide (e.g., bacteriocin) according to some embodiments herein. The method can comprise (a) translating a candidate antimicrobial peptide (e.g., candidate bacteriocin) encoding an antimicrobial peptide (e.g., candidate bacteriocin) in vitro in a translation solution, so that the translation solution thus comprises the candidate antimicrobial peptide (e.g., candidate bacteriocin) 105. The method can comprise (b) combining the candidate antimicrobial peptide (e.g., candidate bacteriocin) and a microbial organism in a solution environment 110, for example by mixing microdroplets comprising the candidate antimicrobial peptide (e.g., candidate bacteriocin) and microbial organism, respectively to form a microdroplet of the solution environment. The method can comprise (c) culturing the microbial organism and the candidate antimicrobial peptide (e.g., candidate bacteriocin) in the solution environment under selected culture conditions 115 as described herein, for example temperature, pH, nutrient content, microbiota flora, in vivo conditions, and/or industrial feedstock. The method can comprise (d) detecting inhibition of growth and/or reproduction, or a lack thereof, of the microbial organism in the solution environment 120. The detecting can be performed as described herein. The method can comprise (e) selecting the candidate nucleic acid when the quantity of microbial organism in the solution environment indicates inhibition of growth and/or reproduction of the microbial organism in the solution environment under the selected culture conditions 125, for example if the quantity of, or growth rate of the microbial organism does not exceed a reference level as described herein. The method can comprise (f) producing a variant nucleic acid of the selected candidate nucleic acid, the variant nucleic acid encoding a variant of the candidate antimicrobial peptide 130, for example by chemical synthesis, and/or by mutagenesis or degenerate polymerase as described herein. The method can comprise comparing the detected inhibition of growth and/or reproduction, or a lack thereof, to a predetermined level of inhibition of growth and/or reproduction 135. If the predetermined level of inhibition of growth and/or reproduction of the microbial organism in the solution environment under the selected culture conditions is again not achieved 140, the method can comprise repeating the cycle 145 using a different candidate antimicrobial peptide (e.g., different candidate bacteriocin). In some embodiments, the sequence of the different candidate antimicrobial peptide (e.g., different candidate bacteriocin) is selected based on indexing the sequence information of the candidate antimicrobial peptide to the detected of inhibition of growth and/or reproduction (or the lack thereof) for the microbial organism. If the predetermined level of inhibition of growth and/or reproduction of the microbial organism in the solution environment under the selected culture conditions is achieved 150, the method can indicate that a desired antimicrobial peptide (e.g., bacteriocin) has been engineered 155. In some embodiments, the cycle is repeated 145 using iteratively different candidate antimicrobial peptides (e.g., different candidate bacteriocins) until the predetermined level of inhibition of growth and/or reproduction of the microbial organism in the solution environment under the selected culture conditions is achieved 150. This result can indicate that the desired antimicrobial peptide (e.g., bacteriocin) has been engineered 155. In some embodiments, one or more of the steps is performed in a different order than is shown, one or more additional steps are added, or one or more step is omitted. For example, in some embodiments, (e) is omitted.

In some embodiments, the method comprises producing the variant nucleic acid based on the indexed sequence of the candidate nucleic acid. It is contemplated that such indexing can advantageously identify antimicrobial peptide (e.g., bacteriocin) structures that positively (or negatively) correlate with inhibition of growth and/or reproduction of a microbial organism in a solution environment, and thus can guide the selection of variants of candidate antimicrobial peptides (e.g., bacteriocins) so as to select sequences, motifs, and/or other structures that are predicted to yield enhanced inhibition of growth and/or reproduction of the microbial organism in the solution environment. Accordingly, with continued reference to FIG. 1, some embodiments of the method comprise one or more optional steps 160. In some embodiments, a sequence of the selected candidate nucleic acid is obtained 165. In some embodiments, the sequence is obtained 165 after (f) 130. In some embodiments, the method comprises indexing the sequence to the detected inhibition of growth and/or reproduction of the microbial organism in the solution environment of (d) 170. The indexing can be performed as described herein, for example using a processor as described herein. In some embodiments, (f) comprises producing the variant nucleic acid based on the indexed sequence of the candidate nucleic acid 175. The variant nucleic acid can be produced as described herein, for example by chemical synthesis of a variant nucleic acid encoding a specified variant of a candidate antimicrobial peptide (e.g., bacteriocin). Accordingly, in some embodiments, the producing the selecting of the sequence of the variant candidate antimicrobial peptide (e.g., bacteriocin) (and the variant nucleic acid sequence) is based on the indexed sequence information comprises machine learning, such as automated machine learning.

It is further contemplated that sequences of candidate antimicrobial peptides (e.g., bacteriocin) that do not yield inhibition of growth and/or reproduction of the microbial organism in the sequence environment (or that yield inhibition of growth and/or reproduction that falls short of a predetermined level) can inform the selection of variants of candidate antimicrobial peptides (e.g., bacteriocin). In accordance with some embodiments, the method comprises obtaining the sequence of an other candidate nucleic acid (encoding an other candidate antimicrobial peptide) for which was detected a lack of inhibition of growth and/or reproduction of the microbial organism in another solution environment under the selected culture conditions (in addition to an absence of inhibition of growth and/or reproduction, this method also contemplates other candidate nucleic acids for which inhibition of growth and/or reproduction of the microbial organism is less than that of the candidate nucleic acid, and/or falls short of a predetermined level). The method can further comprise indexing the sequence of the other candidate nucleic acid to the lack of inhibition of growth and/or reproduction of the microbial organism. In accordance with this method, producing a variant nucleic acid comprises producing the variant nucleic acid of the selected candidate nucleic acid based on the indexed sequence of the other candidate nucleic acid. Thus, the inhibition of growth and/or reproduction, or a lack thereof, is indexed, and the indexed information is used to produce a variant nucleic acid. In some embodiments the sequence of the other candidate nucleic acid is obtained if the other candidate nucleic acid produces a lower level of inhibition of growth and/or reproduction of the microbial organism in the solution environment under the selected culture conditions than the candidate nucleic acid. In some embodiments, producing the variant nucleic acid comprises one or more of amplifying the selected candidate nucleic acid with a degenerate polymerase; amplifying the selected candidate nucleic acid in the presence of a degenerate primer; amplifying the selected candidate nucleic acid in the presence of a degenerate nucleotide; and mutagenesis reaction on the selected candidate nucleic acid.

It is further contemplated that if a candidate antimicrobial peptide (e.g., bacteriocin) is determined to yield a suitable inhibition of reproduction and/or growth of the microbial organism in the solution environment, the method of some embodiments can further comprise confirming the inhibition of reproduction and/or growth by repeating at least portions of the method using the same candidate antimicrobial peptide (e.g., bacteriocin). In some embodiments, the method comprises confirming the inhibition of growth and/or reproduction of the microbial organism in the solution environment by the candidate antimicrobial peptide (e.g., bacteriocin) after (e). The confirming can comprise repeating (a)-(d) for the candidate nucleic acid or a copy thereof. The inhibition of growth and/or reproduction can be confirmed when, in the repeat of (d), the quantity of microbial organism in the solution environment indicates inhibition of growth and/or reproduction of the microbial organism in the solution environment under the selected culture conditions.

In accordance with the methods, systems and kits of some embodiments described herein, an engineered antimicrobial peptide (e.g., bacteriocin) and a candidate antimicrobial peptide (e.g., bacteriocin) of an early iteration of a method as described herein each have a potency. A “potency” refers to an amount by which the engineered antimicrobial peptide (e.g., bacteriocin) or candidate antimicrobial peptide (e.g., bacteriocin) inhibits growth and/or reproduction of a microbial organism, for example a change in growth or reproduction rate, or a change in the number of microbial organisms in the solution environment. In some embodiments, the potency of the engineered antimicrobial peptide (e.g., bacteriocin) is greater than the potency of the candidate antimicrobial peptide (e.g., bacteriocin) (from an earlier iteration of the method). In some embodiments, the engineered antimicrobial peptide (e.g., bacteriocin) has a greater potency than the candidate antimicrobial peptide (e.g., bacteriocin) across a range of culture conditions and/or against a range of microbial organism strains and/or species.

It is contemplated that many industrial, pharmaceutical, cosmetic, and microbiota environments will contain more than one type of microbial organism that can be regulated. Accordingly, in methods, systems, and kits of some embodiments, the candidate antimicrobial peptide (e.g., bacteriocin) inhibits the growth and/or reproduction of more than one type of microbial organism, such as more than one species and/or strain of microbial organism. It is further contemplated that it can be desirable in many industrial, pharmaceutical, cosmetic, and microbiota environments to inhibit the growth and/or reproduction of one or more kinds of undesired microbial organisms (e.g., pathogenic and/or contaminating strains and/or species), while preserving one or more microbial organisms unaffected by the antimicrobial peptides (e.g., bacteriocin) (e.g., commensal, industrially useful, and/or otherwise desirable microbial organisms may be preserved). Accordingly, in some embodiments, the candidate antimicrobial peptide (e.g., bacteriocin) does not inhibit the growth and/or reproduction of one or more microbial organisms in the solution environment. In some embodiments, the candidate antimicrobial peptide (e.g., bacteriocin) inhibits the growth and/or reproduction of at least one type of microbial organism in the solution environment, but does not inhibit the growth and/or reproduction of one or more other types of microbial organisms in the solution environment. In some embodiments, one candidate antimicrobial peptide (e.g., bacteriocin) inhibits the growth and/or reproduction of a microbial organism, and another candidate antimicrobial peptide (e.g., bacteriocin) does not inhibit the growth and/or reproduction of the microbial organism. In some embodiments, a candidate antimicrobial peptide (e.g., bacteriocin) inhibits the growth and/or reproduction of a microbial organism, but does not inhibit the growth and/or reproduction of a second microbial organism.

In some embodiments of the method one or more portions of the method are repeated and/or performed in a different order than is shown in FIG. 1. In some embodiments of the method, (b) is repeated. In some embodiments of the method, repeating (b) of (a)-(f) comprises combining the translation solution with a different strain or species of microbial organism than that of a previous iteration of (b). For example, it may be desired to engineer an antimicrobial peptide (e.g., bacteriocin) to be effective against one microbial organism and also another microbial organism. In some embodiments, repeating (b) of (a)-(f) comprises combining the translation solution with the same species or strain of microbial organism as a previous iteration of (b). For example, multiple iterations of the method to produce and test multiple variant candidate antimicrobial peptides (e.g., variant candidate bacteriocins) may result in an engineered antimicrobial peptide (e.g., engineered bacteriocins) with enhanced properties against a microbial organism compared to just one iteration of (a)-(f). In some embodiments, (c) is repeated. In some embodiments, repeating (c) of (a)-(f) comprises culturing the microbial organism in a different solution environment and/or under different culture conditions than a previous iteration of (c). For example, it may be desired to engineer an antimicrobial peptide (e.g., bacteriocin) to be effective in multiple solution environments. In some embodiments, repeating (c) of (a)-(f) comprises culturing the microbial organism in the same culture environment as a previous iteration of (c).

In some embodiments of the method, the candidate nucleic acid comprises DNA. In some embodiments, the method comprises transcribing the candidate nucleic acid (e.g., DNA). The candidate nucleic acid can be transcribed in a transcription solution as described herein. In the method of some embodiments, the translation solution comprises a transcription solution, in which the translation solution is configured for transcription and translation of the candidate nucleic acid (e.g., DNA). In the method of some embodiments, the translation solution comprises a translation reagent, such as a ribosome. In the method some embodiments, the translation solution comprises one or more post-translational modification enzymes. In some embodiments, the translation solution further comprises a substrate, and the candidate nucleic acid is immobilized on the substrate. In the method of some embodiments, the translation solution and/or the solution environment is microliter-scale. In the method of some embodiments, the translation solution and/or the solution environment has a volume of 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl. In some embodiments, the solution environment is a microdrop. In methods, kits, and systems of some embodiments, the solution environment is hydrophilic. In methods, kits, and systems of some embodiments, the solution environment is hydrophobic. In methods, kits, and systems of some embodiments, the solution environment comprises a hydrogel. In methods, kits, and systems of some embodiments, the solution environment is hydrophilic. In methods, kits, and systems of some embodiments, the solution environment does not comprise any cells comprising nucleic acid encoding the antimicrobial peptide.

It is further contemplated that in methods, kits, and systems of some embodiments, two or more antimicrobial peptides (e.g., bacteriocin) are co-engineered to inhibit growth and/or reproduction of the microbial organism under the selected culture conditions. In some embodiments of the method, the candidate nucleic acid encodes two or more different candidate antimicrobial peptides (e.g., candidate bacteriocins) so that a cocktail of candidate antimicrobial peptides is encoded. For example, the two or more different candidate nucleic acids can each have their own promoter, and/or the downstream candidate nucleic acid can contain an IRES, and/or the candidate nucleic acid can comprise a sequence encoding 2A or a proteolytic site between the sequences encoding the candidate antimicrobial peptides (e.g., candidate bacteriocins). Accordingly, the solution environment in the method, system, and/or kit of some embodiments can comprise two or more candidate antimicrobial peptides (e.g., candidate bacteriocins). Additionally, the variant nucleic acid can encode variants of at least one of the two or more candidate antimicrobial peptides (e.g., bacteriocin), for example both of the candidates. Thus, two or more antimicrobial peptides (e.g., bacteriocin) can be co-engineered to inhibit growth and/or reproduction of the microbial organism under the selected culture conditions. For example, more than one candidate nucleic acids may be co-engineered together to have synergistic effects. In some embodiments, the translation solution comprises no more than one candidate nucleic acid sequence encoding a candidate antimicrobial peptide (e.g., bacteriocin). In some embodiments of the method, the candidate antimicrobial peptide (e.g., bacteriocin) comprises a chimeric protein.

In some embodiments of the method, a candidate nucleic acid is immobilized on a substrate, for example a bead as described herein. Example of suitable substrates suitable for methods, systems, and kits of some embodiments include a bead, nanoparticle, well, membrane, nitrocellulose, PVDF, nylon, acetate derivative, matrix, pore, plastic, metal, glass, polymer, polysaccharide, or paramagnetic compound. In some embodiments, the method comprises immobilizing the candidate nucleic acid on the substrate. In some embodiments, the method comprises producing the variant nucleic acid immobilized on another substrate, which this is the same as, or different from the substrate that the candidate nucleic is or was immobilized on.

In some embodiments of the method, a library of candidate nucleic acids is screened. For example, two or more candidate nucleic acids of a library can be screened in parallel (for example, in different microfluidic devices, or in different portions of the same microfluidic device), and/or can be screened in serial (for example, on the same microfluidic devices, one-at-a-time). In some embodiments, producing the variant nucleic acid comprises producing a library of variant nucleic acids, and the method further comprises performing (a)-(e) on the library of variant nucleic acids. For example, several antimicrobial peptides (e.g., bacteriocins) may be engineered at the same time, or several variant antimicrobial peptides (e.g., bacteriocins) may be tested at the same time, and one(s) that yield inhibition of growth and/or reproduction of a microbial organism are selected for producing variant nucleic acids. By way of example, in some embodiments, a library of candidate nucleic acids (or variant nucleic acids) comprises at least 5, 10, 100, 500,1000,5000, 10,000, 50,000, 100000 candidate nucleic acids (or variant nucleic acids).

In some embodiments, the method is performed in a microfluidic system as described herein.

### Microfluidic Systems for Engineering Antimicrobial Peptides (e.g., Bacteriocins)

A microfluidic system can be useful to determine if a candidate antimicrobial peptide (e.g., bacteriocin) inhibits growth and/or reproduction of a microbial organism in a solution environment under selected culture conditions, and to engineer an antimicrobial peptide (e.g., bacteriocin) in accordance with some embodiments herein. In some embodiments, multiple components of the system work together to produce engineered antimicrobial peptides (e.g., bacteriocin) with desired activities that are tested in one or more of industrial, pharmaceutical, or physiological (e.g., microbiota) culture conditions.

In some embodiments, a microfluidic system for engineering an antimicrobial peptide (e.g., bacteriocin) is provided. In some embodiments, the microfluidic system comprises a transcription station configured to perform in vitro transcription of a candidate nucleic acid. The transcription station can comprise transcription reagent. The microfluidic system can further comprise a translation station in fluid communication with the transcription station. The translation station can be configured to perform in vitro translation. The translation station can further comprise a translation reagent. The system can further comprise a culture station in fluid communication with the translation station and configured to culture a microbial organism in a solution environment comprising the microbial organism, a candidate nucleic acid encoding a candidate antimicrobial peptide (e.g., candidate bacteriocin), and the candidate antimicrobial peptide. The culture can be under selected culture conditions as described herein. The system can further comprise a detector in fluid communication with the culture station. The detector can be configured to detect inhibition of growth and/or reproduction (or a lack thereof) of the microbial organism in the solution environment. The system can further comprise a variant station configured to produce a variant nucleic acid of the nucleic acid encoding the candidate antimicrobial peptide (e.g., candidate bacteriocin). The variant station is in fluid communication with the transcription station. In some embodiments, the variant station is in data communication with the detector (either directly, or via processor). Accordingly, the system (for example via the processor) can be configured to index (for example, via the processor) the sequence information to the detection of inhibition of growth and/or reproduction or the lack thereof for the microbial organism.

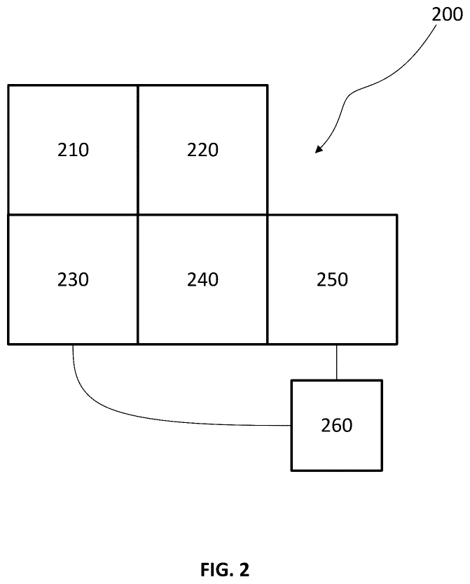

FIG. 2 is a schematic diagram of a system 200 for engineering antimicrobial peptides (e.g., bacteriocin) according to some embodiments herein. The system can comprise a transcription station 210 and/or a translation station 220 as provided herein (which can be comprised by a single transcription/translation station, or can be separate stations). The translation station 220 can be in fluid communication with the transcription station 210. The system can further comprise a culture station 240 as described herein. The culture station 240 can be in fluid communication with the translation station 220 (for example, directly, and/or via a transcription station). The system comprise a detector 250 as described herein. The detector 250 can be in fluid communication with the culture station 240, and can be configured to detect inhibition of growth and/or reproduction of a microbial organism, or a lack thereof. The system can further comprise a variant station 230 in fluid communication with the transcription station 210 and/or translation station 220. In some embodiments, the variant station comprises a sequencing module as described herein. The system of some embodiments further comprises a processor 260. In some embodiments, the processor 260 is in data communication with the detector 250 and/or the variant station 230. For example, the processor can be configured to index sequence information of a candidate antimicrobial peptide (e.g., bacteriocin) to the detection of inhibition of growth and/or reproduction or the lack thereof for the microbial organism as described herein. In some embodiments, the detector 250 detects inhibition of growth or reproduction of the microbial organism in the solution environment in the culture station 240. In some embodiments, the detector 250 detects inhibition of growth or reproduction of the microbial organism at a location outside of the culture station 240. In some embodiments, the microfluidic system comprises a microdrop system, and is configured to move mircrodrops comprising candidate nucleic acids, candidate antimicrobial peptides (e.g., bacteriocin) and/or microbial organisms to the applicable station(s), and/or to move the applicable station(s) to the microdrop. In some embodiments, the microfluidic system comprise a microfluidic channel, for example a network of microfluidic channels.

In some embodiments, two or more of the transcription station, the translation station, the culture station, and/or the variant station are comprised within discrete chambers that are separate from each other. In some embodiments, the transcription station and the translation station are the same station or overlap with each other (e.g., a single transcription/translation station). In some embodiments, two or more of the transcription station, the translation station, the culture station, and the variant station, are comprised within a single chamber. Optionally, the single chamber can be configured to expel a first set of reagents and subsequently receive a second set of reagents that is different from the first set so that a single location can be used for two or more functions (for example, transcription and translation, and/or culture and detection).

### Transcription Stations

Some embodiments of the microfluidic system comprise a transcription station. In some embodiments, the transcription station is configured to perform in vitro transcription. In some embodiments, the transcription station comprises a transcription solution as described herein. In some embodiments, the transcription station comprises one or more transcription reagents. In some embodiments, the transcription station is part of a single transcription/translation station. In some embodiments, the transcription station is the same station as the translation station. In some embodiments, the transcription station is separate from the translation station, for example separated by a wall, barrier, valve, or the like.

### Translation Stations

The microfluidic system of some embodiments comprises a translation station. In some embodiments, the translation station is configured to perform in vitro translation of a candidate nucleic acid encoding a candidate antimicrobial peptide (e.g., candidate bacteriocin). In some embodiments, the translation station comprises a translation solution as described herein. In some embodiments, the translation station comprises one or more translation reagents. In some embodiments, the translation station comprises one or more post-translational modification enzymes.

In some embodiments, the translation station is in fluid communication with a substrate comprising the candidate nucleic acid immobilized thereon, for example when variant nucleic acids are synthesized on a substrate in the variant station. In some embodiments, the substrate comprises a bead, nanoparticle, well, membrane, nitrocellulose, PVDF, nylon, acetate derivative, matrix, pore, plastic, metal, glass, polymer, polysaccharide, or paramagnetic compound.

In some embodiments, the translation station comprises a chamber (e.g., an in vitro translation chamber) that is microliter-scale and/or the solution environment is microliter-scale. In some embodiments, the chamber of the translation station has a volume of about 1 μl-1000 μl, 1 μl-50 μl, 1 μl-100 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl.

In some embodiments, the translation station comprises a mixture of different candidate antimicrobial peptides (e.g., candidate bacteriocins).

### Culture Stations

Some embodiments of the microfluidic system comprise a culture station. In some embodiments, the culture station is configured to culture one or more microbial organisms in a culture environment under selected culture conditions as described herein, for example conditions of an industrial process, chemical or pharmaceutical manufacturing process, fermentation process, and/or a microbiota (such as a mammalian microbiota). In some embodiments, the culture station comprises a solution environment as described herein. In some embodiments, the culture station is configured to culture a microbial organism in a solution environment. In some embodiments, the solution environment comprises the microorganism, a candidate nucleic acid encoding a candidate antimicrobial peptide (e.g., candidate bacteriocin), and the candidate antimicrobial peptide under selected culture conditions.

In some embodiments, the selected culture conditions of the culture station comprise conditions of an industrial process, pharmaceutical manufacturing process, or microbiota (e.g., mammalian microbiota) as described herein.

In some embodiments, the solution environment in the culture station has a volume of about 1 μl-1000 μl, 1 μl-50 μl, 1 μl-500 μl, 1 μl-900 μl, 50 μl-100 μl, 50 μl-500 μl, 50 μl-1000 μl, 100 μl-200 μl, 100 μl-500 μl, 100 μl-1000 μl, 200 μl-500 μl, 200 μl-1000 μl, 500 μl-900 μl, or 500 μl-1000 μl. In some embodiments, the solution environment is a microdrop.

### Detectors

The microfluidic system of some embodiments comprises a detector as described herein. The detector can be configured to detect inhibition of growth and/or reproduction of a microbial organism in solution environment as described herein. In some embodiments, the detector comprises an optical detector such as a spectrophotometer. The spectrophotometer can be configured to measure an optical density (for example at OD₆₀₀), which can indicate an amount (and/or rate of change in an amount) of microbial organisms in the solution environment. In some embodiments, the detector comprises a camera. In some embodiments, the detector is in fluid communication with a culture station as described herein. In some embodiments, the detector is configured to detect inhibition of growth and/or reproduction of a microbial organism in a culture station as described herein. In some embodiments, the detector is configured to detect growth and/or reproduction of a microbial organism in a culture station as described herein, but is not in fluid communication with the culture station. In some embodiments, the detector is configured to detect a lack of growth and/or reproduction of a microbial organism in a culture station. In some embodiments, the detector is configured to detect inhibition of growth and/or reproduction, or a lack thereof, of a microbial organism in a culture station. In some embodiments, the detector is connected to a processor.

### Variant Stations

The microfluidic system of some embodiments comprises a variant station. In some embodiments, the variant station is configured to produce a variant nucleic acid of a nucleic acid encoding a candidate antimicrobial peptide (e.g., bacteriocin) as described herein. In some embodiments, the variant station is in data communication with a processor as described herein. The variant station can produce a selected variant nucleic acid encoding a variant of the candidate antimicrobial peptide (e.g., bacteriocin) based on an index of sequence information of the candidate antimicrobial peptide (e.g., bacteriocin) to inhibition of growth and/or reproduction of the microbial organism (or a lack thereof). The indexing can be performed by a processor as described herein. In some embodiments, the variant station is configured to perform a mutagenesis reaction to produce the variant nucleic acid. In some embodiments, the variant station comprises a degenerate polymerase, a degenerate primer, and/or a degenerate nucleotide, which can produce the variant nucleic acid.

In some embodiments, the variant station comprises a sequencing module configured to obtain sequence information from a candidate nucleic acid. The sequencing module can perform sequencing as described herein. In some embodiments, the microfluidic system is configured to index the sequence information to a detection of inhibition of growth and/or reproduction or the lack thereof for a microbial organism in the solution environment. In some embodiments, the microfluidic system is configured to select a sequence of the variant nucleic acid based on the indexed information by machine learning, for example automatic machine learning. In some embodiments, a processor is configured to index the sequence information, and/or select the sequence of the variant nucleic acid (for example, based on a selected sequence of a variant of the candidate antimicrobial peptide (such as a candidate bacteriocin).

In some embodiments, the variant station produces the variant nucleic acid only if the detector detects an inhibition of growth and/or reproduction of the microbial organism in the solution environment.

### Kits

In some embodiments, a kit for engineering an antimicrobial peptide (e.g., bacteriocin) is provided. In some embodiments, the kit comprises a candidate nucleic acid encoding a candidate antimicrobial peptide (such as a candidate bacteriocin), and a microfluidic system as described herein. In some embodiments, the kit comprises a library of candidate nucleic acids. In some embodiments, the kit comprises a microbial organism. In some embodiments, the kit further comprises instructions that the kit is to be used for engineering an antimicrobial peptide (e.g., bacteriocin).

### EXAMPLE 1

### Screening and Selection of Variant Antimicrobial Peptides With Desired Properties

A library of candidate nucleic acids comprising point mutations of a nucleotide sequence encoding wild-type microcin V bacteriocin (SEQ ID NO: 65) is engineered by the following method. The library is prepared by introducing point mutations in SEQ ID NO: 65 with a degenerate DNA polymerase. Each candidate nucleic acid of the library comprises a point mutations of SEQ ID NO: 65. Each candidate nucleic acid is transcribed and translated in vitro in a translation solution that includes reagents for in vitro transcription and in vitro translation. After the in vitro transcription and translation is complete, each translation solution is a microdrop of about 10 μl in volume, which comprises a candidate antimicrobial peptide (that is a variant of microcin V).

Each translation solution comprising a candidate antimicrobial peptide is combined with a microliter-scale solution environment inoculated with Salmonella enterica. The solution environment is designed to mimic an industrial process involved in the production of chicken broth, and contains chicken broth at room temperature and a pH of 7.2, with preservatives and additives specific to the chicken broth. Once combined, the microliter-scale solution environment containing the Salmonella enterica and candidate antimicrobial peptide is a microdrop having a volume of about 500 μl.

The solution environment is incubated at room temperature for 24 hours. Salmonella growth is monitored during the incubation period using a spectrophotometer, and a final measurement of Salmonella density is determined at the end of the incubation period. The measurements include a spectrophotometric reading at OD₆₀₀. The Salmonella growth rate in each solution environment is compared to Salmonella growth in a control solution environment that includes the wild-type microcin V antimicrobial peptide without any point mutations (encoded by SEQ ID NO: 65). Candidate nucleic acids encoding candidate antimicrobial peptides (e.g., bacteriocin) in solution environments with 50% or less of the Salmonella growth rate of the control solution environment.

The selected candidate nucleic acids are amplified by PCR with a degenerate DNA polymerase to produce variant nucleic acids. The variant nucleic acids are then transcribed and translated in vitro, and variant antimicrobial peptides (variant bacteriocins) produced by the in vitro transcription and translation of the variant nucleic acids are combined with solution environments containing Salmonella enterica and chicken broth. The variant nucleic acids and variant antimicrobial peptides (variant bacteriocins) are considered candidate nucleic acids and candidate antimicrobial peptides (candidate bacteriocins) in another round of screening selection. In another round of screening selection, the solution environments containing the new candidate antimicrobial peptides are incubated, and Salmonella growth is monitored as with the prior solution environments containing the prior candidate antimicrobial peptides (candidate bacteriocins), and the new candidate antimicrobial peptides (candidate bacteriocins) that limit Salmonella growth the most (for example by at least 75% compared to the control wild-type microcin V bacteriocin) are selected. By this method the microcin V bacteriocin is engineered to more effectively inhibit Salmonella growth. More rounds of making variants, and screening and selection are performed if desired to further engineer the antimicrobial peptide (bacteriocin) and enhance its properties.

### EXAMPLE 2

### Engineering Antimicrobial Peptides With Machine Learning

A method similar to that of Example 1 is used to engineer the library of candidate nucleic acids comprising point mutations of the microcin V bacteriocin, except that machine learning is used to select and modify the bacteriocins. Additionally, the nucleotide sequence of each candidate nucleic acid is obtained by DNA sequencing.

The library is screened, and Salmonella growth is measured in each solution environment comprising a candidate antimicrobial peptides (candidate bacteriocins). A processor stores information relating to the Salmonella growth in each solution environment in a database. Information relating to the growth inhibition is stored for each candidate antimicrobial peptide (e.g., bacteriocin) regardless of the amount of growth inhibition it exerted on the Salmonella. The stored information is tabulated by the processor, and is indexed with the sequence information for each point mutation in microcin V. The indexing by the processor associates each point mutation with the detected level of growth inhibition (or lack thereof) for that point mutant. The processor performs structure function correlation, so as to select point mutations that yielded inhibition of growth and/or reproduction of the Salmonella, while avoiding those that did not. Thus, the processor selects variants of the candidate antimicrobial peptide (e.g., bacteriocin), and corresponding sequences of variant nucleic acids to be tested in further iterations of the screening. Some of the variant nucleic acids include multiple point mutations. Thus, the processor selects candidate antimicrobial peptide (e.g., bacteriocin) sequences by automated machine learning.

Variant nucleic acids with the sequences designed by the processor via machine learning are then chemically synthesized, and transcribed and translated in vitro to produce variant antimicrobial peptides (e.g., bacteriocins). The variant antimicrobial peptides (e.g., bacteriocin) produced by the in vitro transcription and translation are screened in solution environments containing Salmonella. The Salmonella growth is monitored, and a processor again performs a structure function-analysis based on the indexing of variant sequences to growth inhibition information for each variant. Information from the first analysis is used to inform this second structure function analysis, and thus the processor learns an optimized sequence for inhibiting Salmonella growth in the solution environment. Thus, the processor engineers a new antimicrobial peptide (e.g., bacteriocin) that is engineered to inhibit Salmonella growth in the solution environment under culture conditions of an industrially-relevant process.

### EXAMPLE 3

### Microdroplet Apparatus for Screening and Selection of Variant Antimicrobial Peptides

A kit containing a microfluidic system is provided. The microfluidic system comprises a microdroplet apparatus configured to perform in vitro transcription and translation of a candidate nucleic acid encoding a candidate antimicrobial peptide such as a candidate bacteriocin) on a bead in a first microdroplet. The microdroplet apparatus is configured to contact the first microdroplet (containing the translated candidate antimicrobial peptide such as a candidate bacteriocin) with a second microdroplet that includes a bacteria. Contacting the first microdroplet with the second microdroplet produces a combined microdroplet that comprises a culture environment comprising the combined contents of the first and second microdroplets. The microfluidic apparatus is configured to culture the third microdroplet for a period of time at selected culture conditions (for example 14 hours at 37° C.). The microdroplet device includes a detector configured to measure reproduction of the bacteria, or a lack thereof, in the third microdroplet. The microdroplet device includes a processor configured to receive and process information relating to the detected reproduction, or lack thereof. Thus, the microfluidic system is suitable for engineering an antimicrobial peptide (e.g., bacteriocin) in accordance with some embodiments herein.

In at least some of the embodiments described herein, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1. A method of engineering an antimicrobial peptide, the method comprising:
(a) translating a candidate nucleic acid encoding a candidate antimicrobial peptide in vitro in a translation solution, whereby the translation solution comprises the candidate antimicrobial peptide;
(b) combining the candidate antimicrobial peptide and a microbial organism in a solution environment;
(c) culturing the microbial organism and the candidate antimicrobial peptide in the solution environment under selected culture conditions;
(d) detecting inhibition of growth and/or reproduction, or a lack thereof, of the microbial organism in the solution environment;
(e) selecting the candidate nucleic acid upon detection of inhibition of growth and/or reproduction of the microbial organism in the solution environment under the selected culture conditions;
(f) producing a variant nucleic acid of the selected candidate nucleic acid, the variant nucleic acid encoding a variant of the candidate antimicrobial peptide; and
repeating (a)-(f) using one or more successive variant nucleic acids as the candidate nucleic acid, until a predetermined level of inhibition of growth and/or reproduction of the microbial organism in the solution environment under the selected culture conditions is achieved, whereby the antimicrobial peptide has been engineered,
wherein the candidate nucleic acid encodes two or more different candidate antimicrobial peptides, whereby the solution environment comprises two or more candidate antimicrobial peptides, and
wherein the variant nucleic acid encodes variants of at least one of the two or more candidate antimicrobial peptides, and
whereby two or more antimicrobial peptides are co-engineered to inhibit growth and/or reproduction of the microbial organism under the selected culture conditions.

2.-33. (canceled)

34. A microfluidic system for engineering an antimicrobial peptide, comprising:
a transcription station configured to perform in vitro transcription, the transcription station comprising a transcription reagent;
a translation station in fluid communication with the transcription station, the translation station configured to perform in vitro translation, and comprising a translation reagent;
a culture station in fluid communication with the translation station and configured to culture a microbial organism in a solution environment comprising the microbial organism, a candidate nucleic acid encoding a candidate antimicrobial peptide , and the candidate antimicrobial peptide under selected culture conditions;
a detector in fluid communication with the culture station, and configured to detect inhibition of growth and/or reproduction, or a lack thereof, of the microbial organism in the solution environment; and
a variant station configured to produce a variant nucleic acid of the nucleic acid encoding the candidate antimicrobial peptide,
wherein the variant station is in fluid communication with the transcription station.

35. The microfluidic system of claim 34, further comprising a processor,
wherein the variant station further comprises a sequencing module configured to obtain sequence information from the candidate nucleic acid,
the processor configured to index the sequence information to the detection of inhibition of growth and/or reproduction or the lack thereof for the microbial organism.

36. The microfluidic system of claim 35, wherein the processor is configured to select a sequence of the variant nucleic acid based on the indexed information by machine learning.

37. The microfluidic system of claim 34, wherein the variant station produces the variant nucleic acid only if the detector detects an inhibition of growth and/or reproduction of the microbial organism in the solution environment.

38. The microfluidic system of claim 34, wherein the variant station comprises a degenerate polymerase.

39. The microfluidic system of claim 34, wherein two or more of the transcription station, the translation station, the culture station, and/or the variant station are comprised within discrete chambers that are separate from each other.

40. The microfluidic system of claim 34, wherein the transcription station and the translation station are the same station or overlap with each other.

41. The microfluidic system of claim 34, wherein two or more of the transcription station, the translation station, the culture station, and the variant station, are comprised within a single chamber.

42. The microfluidic system of claim 41, wherein the single chamber is configured to expel a first set of reagents and subsequently receive a second set of reagents that is different from the first set.

43. The microfluidic system of claim 34, wherein the selected culture conditions of the culture station comprise conditions of an industrial process, pharmaceutical manufacturing process, or mammalian microbiota.

44. The microfluidic system of claim 34, wherein the translation station is in fluid communication with a substrate comprising the candidate nucleic acid immobilized thereon.

45. The microfluidic system of claim 44, wherein the substrate comprises a bead, nanoparticle, well, membrane, nitrocellulose, PVDF, nylon, acetate derivative, matrix, pore, plastic, metal, glass, polymer, polysaccharide, or paramagnetic compound.

46. The microfluidic system of claim 34, wherein the translation station comprises a chamber that is microliter-scale and/or the solution environment is microliter-scale.

47. (canceled)

48. (canceled)

49. The microfluidic system of claim 34, wherein the translation station comprises a mixture of different candidate antimicrobial peptides.

50. The microfluidic system of claim 34, wherein the microbial organism comprises multiple species of microbial organism.

51. The microfluidic system of claim 34, wherein the translation station comprises one or more post-translational modification enzymes.

52. A kit for engineering an antimicrobial peptide, comprising:
a candidate nucleic acid encoding a candidate antimicrobial peptide;
a microfluidic system of claim 34.

53. The kit of claim 52, further comprising a library of candidate nucleic acids.

54. (canceled)

55. (canceled)

56. The microfluidic system of claim 34, wherein the antimicrobial peptide comprises a bacteriocin.

57. (canceled)
